(19) United States
(12) Patent Application Publication
(10) Pub. No.: US 2014/0196168 A1
(43) Pub. Date: Jul. 10, 2014

(54) DROUGHT RESISTANT PLANTS AND METHODS FOR MAKING THE SAME USING TRANSCRIPTIONAL REGULATORS

(71) Applicants: Stefan Björklund, Umea (SE); Celine Davoine, Umea (SE); Nils Elfving, Arnasvall (SE); Ove Nilsson, Umea (SE)

(72) Inventors: Stefan Björklund, Umea (SE); Celine Davoine, Umea (SE); Nils Elfving, Arnasvall (SE); Ove Nilsson, Umea (SE)

(73) Assignee: SweTree Technologies AB, Umea (SE)

(21) Appl. No.: 13/980,024

(22) PCT Filed: Jan. 18, 2012
(86) PCT No.: PCT/SE2012/050037

§ 371 Date: Oct. 8, 2013

Related U.S. Application Data
(60) Provisional application No. 61/433,558, filed on Jan. 18, 2011.

(30) Foreign Application Priority Data
Jan. 18, 2011 (SE) 1150025-3

(51) Int. Cl. C12N 15/82; C12N 15/01; A01H 1/02

(52) U.S. Cl. CPC C12N 15/8273; A01H 1/02; C12N 15/01
USPC 800/270; 800/278; 504/196; 800/276; 800/298; 800/320.2; 800/320.3; 800/306; 800/312; 800/319; 800/316; 800/260

(57) ABSTRACT

A method for producing a genetically modified plant with increased tolerance and/or resistance to water deficit and/or salt as compared to a corresponding non-genetically modified wild type plant is provided. Furthermore, a genetically modified annual or perennial crop plant having increased tolerance and/or resistance to water deficit and/or salt as compared to a corresponding non-genetically modified wild type plant is provided, said plants being capable of achieving a higher plant biomass under conditions of water deficit or soil salt salinity.

### FIELD OF THE INVENTION

### Background

Under field conditions, plant performance in terms of growth, development, biomass accumulation and yield depends on acclimation ability to the environmental changes and stresses. Abiotic environmental stresses, such as drought stress and salinity stress, are major limiting factors of plant growth and productivity. Plants exposed to salt stress or drought conditions typically have low yields of plant material, seeds, fruit and other edible products. Crop losses and crop yield losses of major crops such as rice, maize (corn) and wheat as well as forest trees caused by these stresses represent a significant economic and political factor and contribute to food shortages in many underdeveloped countries. Developing stress-tolerant and/or resistant plants and in particular trees is a strategy that has the potential to solve or mediate at least some of these problems. Drought tolerance and/or resistance is known to be a complex quantitative trait, with no real diagnostic marker. This lack of a mechanistic understanding makes it difficult to design a transgenic approach to improve water or salt stress tolerance and/or resistance.

Despite a loss in yield, plants in general exhibit a remarkable capacity to withstand enormous variations in climate, both seasonal variations and prolonged climate changes; in particular trees that are subjected to very large environmental changes over their life time. This ability to adapt to the environment depends on several signalling pathways and transcription factors that are regulated in response to adverse conditions. They can affect target genes directly to increase the ability to tolerate environmental stress or more indirectly by controlling developmental processes such as vegetative growth or timing of floral transition. Transcription of protein-encoding genes in eukaryotes requires RNA polymerase II (pol II) and a set of five general transcription factors (GTFs) involved in promoter recognition, transcription bubble formation and initiation (1). Pol II also depends on the multiprotein Mediator coactivator complex, which conveys signals from promoter-bound regulatory transcription factors to the pol II/GTFs (2). The Mediator coactivator complex in Arabidopsis thaliana comprises a core of protein subunits, some of which are conserved in other eukaryotes while others are specific for plants (3). One of the former is Med25, which in human cells has been identified for example as the target for the VP16 transcriptional activator protein. Plant Med25 was originally identified as PFT1, a nuclear protein acting in a photoreceptor pathway that induces flowering in response to suboptimal light conditions (4), and subsequently has been identified as a key regulator of the jasmonate signaling pathway and is required for infection of some necrotrophic fungal pathogens (5).

Med18 has also been identified as a subunit of the Arabidopsis thaliana Mediator complex, encoded by At2g22370 (3). Med18 was originally identified in yeast as SrbS, a suppressor of a cold-sensitive phenotype found in yeast expressing a truncated version of the C-terminal domain of the largest pol II subunit (RNA polymerase B; Thompson C M., et al., 1993, Cell 73(7):1361-75). Med18 binds to Med20 and both subunits are encoded by non-essential genes in yeast. They are located in the head module of the Mediator complex which is located most proximal to the pol II in the pol II holoenzyme.

There exists a continuing need to identify genes expressed in stress tolerant plants that have the capacity to modulate stress resistance in its host plant and to other plant species, especially to confer increased tolerance and/or resistance to environmental stress, preferably under conditions of water deficiency and salt stress. It is an object of this invention to provide new methods to confer drought and/or salt stress tolerance and/or resistance in plants or plant cells. It is further an object of this invention to provide genetically modified plants that are more drought and/or salt stress resistant as compared to a corresponding non-genetically modified wild type plant, and to thereby achieve a higher plant biomass.

### SUMMARY OF THE INVENTION

The invention provides a method for producing a genetically modified plant with increased tolerance and/or resistance to water deficit and/or salt as compared to a corresponding non-genetically modified wild type plant, which comprises the following steps:
i. reducing or deleting the amount or activity of a Mediator subunit in a plant cell, a plant or a part thereof,
ii. generating and/or selecting a genetically modified plant with increased tolerance and/or resistance to water deficit and/or salt as compared to a corresponding non-genetically modified wild type plant and growing under conditions which permit the development of the plant, wherein said plant is an annual or perennial crop plant.

The method may further comprise:
iii. selfing or crossing the genetically modified plant with itself or another plant, respectively, to produce seed; and
iv. growing a progeny plant from the seed, wherein the progeny plant has increased tolerance and/or resistance to water deficit and/or salt.

In one embodiment of the method, the subunit is a Med25 polypeptide comprising:
a) an activator-interacting domain comprising three peptides localised in sequential sequence of (a), (b) and (c) in the C-terminal half of said polypeptide, and wherein said peptides are:

[SEQ ID NO: 5];
(a) KY(V/I)KXWEGXLSGQRQGQPV(F/L/I)IX(K/R)(L/M)E
(G/A)(Y/F)
[SEQ ID NO: 6], and
(b): LA(A/S)XWPXXMQIVRLI(S/A)Q(D/E)HMNNKQYVGKADF
LVFR(T/A)(M/L)(N/S)XHGFLXQLQ(E/D)KKL
[SEQ ID NO: 7],
(c): CAVIQLPSQTLLLS (V/M)(S/A)DKAXRLIGMLFPGDMWFKPQ

wherein X is any amino acid. Where two or more amino acids are given as alternatives at a given position, if one of these amino acids is given in bold font, this indicates that it is the most highly conserved amino acid at this position.

In a further aspect of this method, the amino acid sequences of peptides (a), (b) and (c) are at least 80% identical to the corresponding peptide of a Med25 polypeptide having SEQ ID NO: 9.

In a further aspect of this method, the Med25 polypeptide further comprises:
b) a vWF-A domain comprising four peptides localised in sequential sequence of (A1), (A2), (A3) and (A4) in the N-terminal half of said polypeptide, said peptides have an amino acid sequence:

[SEQ ID NO: 1];
A1: (E/D)(G/S/T)TAA(L/M/I)GP(Y/F)VVXXXXX(D/E)Y(L/
V/I)(D/E)(K/E)(I/M)(V/I)R(S/C/Y)
[SEQ ID NO: 2];
A2: (E/D)(L/F)(S/A)(L/I)VX(F/Y)(H/N)XHGX(Y/L)(S/C)
(A/G/S)XXVQR(S/T)(G/A)VVT(K/R)DX(D/S/N)XF(L/F/I)
XWLX(G/A/S)(I/L/M)XFXGGG(F/L)X(D/E)(A/V)(A/S)(I/T)
XEGL(A/S)EAL(K/M)(M/I)(L/F)
[SEQ ID NO: 3]; and
A3: (H/N)C(L/I/V)L(V/I)(A/T)A(S/N/T)NP(Y/H)XLXTPV
(Y/F)
[SEQ ID NO: 4].
A4: AEX(V/L)AXXFXXXX(V/I)SLS(V/I)(V/I)(S/C)PKQLP
(T/K)(L/I)(K/R)X(I/L)(Y/F)(N/T)(A/S)(G/A)K(R/P)NX
(Q/R)XXD(P/L)X(V/L/I)(D/E)

In a further aspect of this method, the Med25 polypeptide has an amino acid sequence having at least 80% amino acid sequence identity to a sequence selected from among SEQ ID NO's: 9, 11, 13, 15, 17, 19, 21, 23, 25, 27, 29, 31, 33, 35, and 37.

In a second embodiment of the method, the subunit is a Med18 polypeptide, wherein the amino acid sequence of the polypeptide is at least 80% amino acid sequence identity to a sequence selected from among SEQ ID NO: 39, 41, 43, 45, 47, 49, 51, 53, 55, 57, 59, 61, 63, 65, 67, 69 and 71.

In a further aspect of the first or second embodiment, the method comprises reducing or deleting the expression of at least one nucleic acid molecule, wherein said molecule is selected from: group (i) a nucleic acid molecule encoding the Med 25 polypeptide or the Med18 polypeptide; or group (ii) a nucleic acid molecule having a nucleic acid sequence selected from among SEQ ID NO's: 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68 and 70.

In a further aspect of the first or second embodiment, the method comprises at least one step selected from among: (a) introducing into at least one plant cell a nucleic acid molecule encoding a ribonucleic acid sequence, which is able to form a double-stranded ribonucleic acid molecule, whereby a fragment of at least 17 nucleotides of said double-stranded ribonucleic acid molecule has a nucleic acid sequence having at least 50% nucleic acid sequence identity to a nucleic acid molecule selected from the group (i) or (ii); (b) introducing into at least one plant cell an RNAi or antisense nucleic acid molecule, whereby the RNAi or antisense nucleic acid molecule comprises a fragment of at least 17 nucleotides with a nucleic acid sequence having at least 50% nucleic acid sequence identity to a nucleic acid molecule selected from the group (i) or (ii) (c) introducing into at least one plant cell a nucleic acid construct capable to recombine with and silence, inactivate, or reduce the activity of an endogenous gene comprising a nucleic acid molecule selected from the group (i) or (ii); and (d) introducing or detecting a non-silent mutation in an endogenous gene comprising a nucleic acid molecule selected from the group (i) or (ii).

In a further aspect of the first or second embodiment of this method, the reducing or deleting of the amount or activity of an Med25 polypeptide or Med18 polypeptide is caused by any one of: (i) a natural or induced mutation in an endogenous gene of the plant cell, the plant or a part thereof, and optionally in combination with ECO-TILLING or TILLING; (ii) T-DNA inactivation of an endogenous gene; (iii) site-directed mutagenesis or directed breeding of an endogenous gene, wherein the endogenous gene comprises a nucleic acid molecule selected from the group (i) or (ii).

In a further aspect of the first or second embodiment, this method comprises: (a) providing a vector comprising: (i) said nucleic acid molecule for introducing into at least one plant cell; (ii) a flanking nucleic acid molecule comprising one or more regulatory elements fused to said nucleic acid molecule, wherein the regulatory elements control expression of said nucleic acid molecule; and (b) transforming at least one cell of said plant with the vector to generate a transformed plant with increased tolerance and/or resistance to water deficit and/or salt as compared to a corresponding non-transformed wild type plant.

In a further aspect of the first or second embodiment of this method, the plant is any one of (a) a monocotyledous crop plant selected from the group consisting of Avena spp; Oryza spp.; Hordeum spp., Triticum spp.; Secale spp.; Brachypodium spp.,; Zea spp.; (b) a dicotyledenous crop plant selected from among Cucumis spp.,; Phaseolus spp., Glycine spp.,; Medicago spp.,; Brassica spp; and Beta spp., (c) a hardwood selected from among acacia, eucalyptus, hornbeam, beech, mahogany, walnut, oak, ash, willow, hickory, birch, chestnut, poplar, alder, maple, sycamore, ginkgo, a palm tree and sweet gum; (d) a conifer selected from among cypress, Douglas fir, fir, sequoia, hemlock, cedar, juniper, larch, pine, redwood, spruce and yew, (e) a fruit bearing woody plant selected from among apple, plum, pear, banana, orange, kiwi, lemon, cherry, grapevine, papaya, peanut, and fig and (f) a woody plant selected from among cotton, bamboo and a rubber plant. In a preferred embodiment, the plant is a tree selected from Poplar and Eucalyptus.

The invention provides a genetically modified annual or perennial crop plant having increased tolerance and/or resistance to water deficit and/or salt as compared to a corresponding non-genetically modified wild type plant, wherein said plant has a reduced amount or activity of a Mediator subunit, and wherein the genome of said plant comprises a genetic modification selected from any one of: i) a non-silent mutation in an endogenous gene comprising a nucleic acid molecule encoding a Med25 polypeptide or a Med18 polypeptide; ii) a transgene inserted into said genome, said transgene comprising a nucleic acid molecule encoding a ribonucleic acid sequence, which is able to form a double-stranded ribonucleic acid molecule, whereby a fragment of at least 17 nucleotides of said double-stranded ribonucleic acid molecule has a homology of at least 50% to a nucleic acid molecule encoding a Med25 polypeptide or a Med18 polypeptide; iii) a mutation in an endogenous gene comprising a nucleic acid molecule encoding a Med25 polypeptide or a Med18 polypeptide, induced by introducing into at least one plant cell a nucleic acid construct able to recombine with and silence, inactivate, or reduce the activity of the endogenous gene, wherein said Med25 has an amino acid sequence having at least 80% amino acid sequence identity to a sequence selected from among SEQ ID NO's: 9, 11, 13, 15, 17, 19, 21, 23, 25, 27, 29, 31, 33, 35, and 37; and wherein said Med18 polypeptide has an amino acid sequence selected from among SEQ ID NO's 39, 41, 43, 45, 47, 49, 51, 53, 55, 57, 59, 61, 63, 65, 67, 69 and 71.

In a further aspect, the genetically modified plant is any one of (a) a monocot selected from the group consisting of Avena spp; Oryza spp.; Hordeum spp., Triticum spp.; Secale spp.; Brachypodium spp.,; Zea spp.; (b) a dicot plant selected from among Cucumis spp.,; Phaseolus spp., Glycine spp.,; Medicago spp.,; Brassica spp; and Beta spp., (c) a hardwood selected from among acacia, eucalyptus, hornbeam, beech, mahogany, walnut, oak, ash, willow, hickory, birch, chestnut, poplar, alder, maple, sycamore, ginkgo, a palm tree and sweet gum; (d) a conifer selected from among cypress, Douglas fir, fir, sequoia, hemlock, cedar, juniper, larch, pine, redwood, spruce and yew, (d) a fruit bearing woody plant selected from among apple, plum, pear, banana, orange, kiwi, lemon, cherry, grapevine, papaya, peanut, and fig and (e) a woody plant selected from among cotton, bamboo and a rubber plant.

In a further aspect, the genetically modified plant having increased tolerance to water deficit and/or salt stress as compared to a corresponding non-genetically modified wild type plant, wherein said plant is a hardwood selected from among poplar and eucalyptus and wherein the genome of said plant comprises a transgene inserted into said genome, said transgene comprising a nucleic acid molecule encoding a ribonucleic acid sequence, which is able to form a double-stranded ribonucleic acid molecule having any one of SEQ ID No: 82, 83, 84 or 84.

In a further aspect, the genetically modified plant is a seed or plant part thereof.

### DETAILED DESCRIPTION

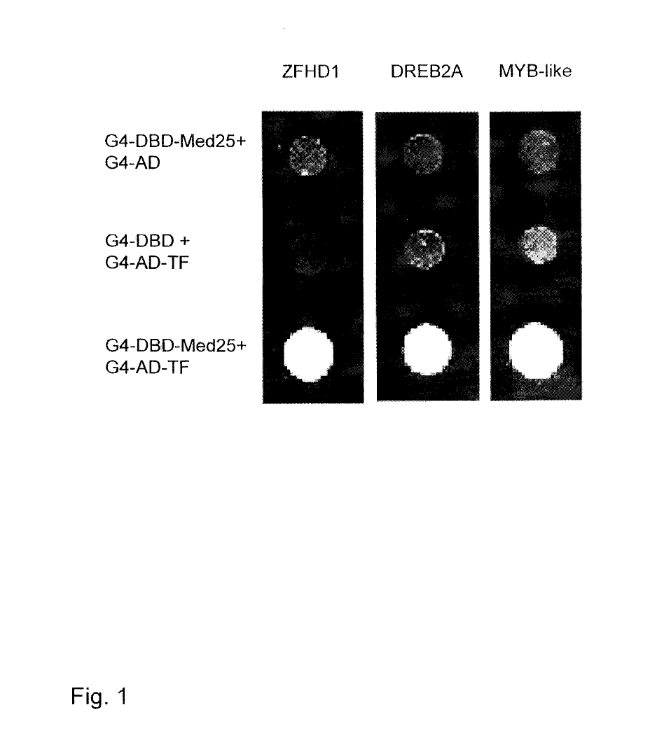

FIG. 1. Yeast-two-hybrid experiments showing interaction between Med25 and each of the transcription factors ZFHD1, DREB2A and MYB-like.

pAD-GAL4-2.1 prey plasmids with (G4-AD-TF) or without (G4-AD) the transcription factors ZFHD1, DREB2A and MYB-like (previously isolated in the two-hybrid screen) were re-transformed into yeast strain AH109 containing the bait plasmid pGBKT7 expressing the Ga14-DNA Binding Domain with, and without, fusion to the Med25₅₅₁₋₆₈₀ amino acid domain (G4-DBD and G4-DBD-Med25). Cells were plated on high stringency media (SDTrp/-Leu/-His/-Ade) and incubated at 30° C. The experiment shows that the interaction is specific between Med25 and the transcription factors which alone do not self-activate the reporter genes.

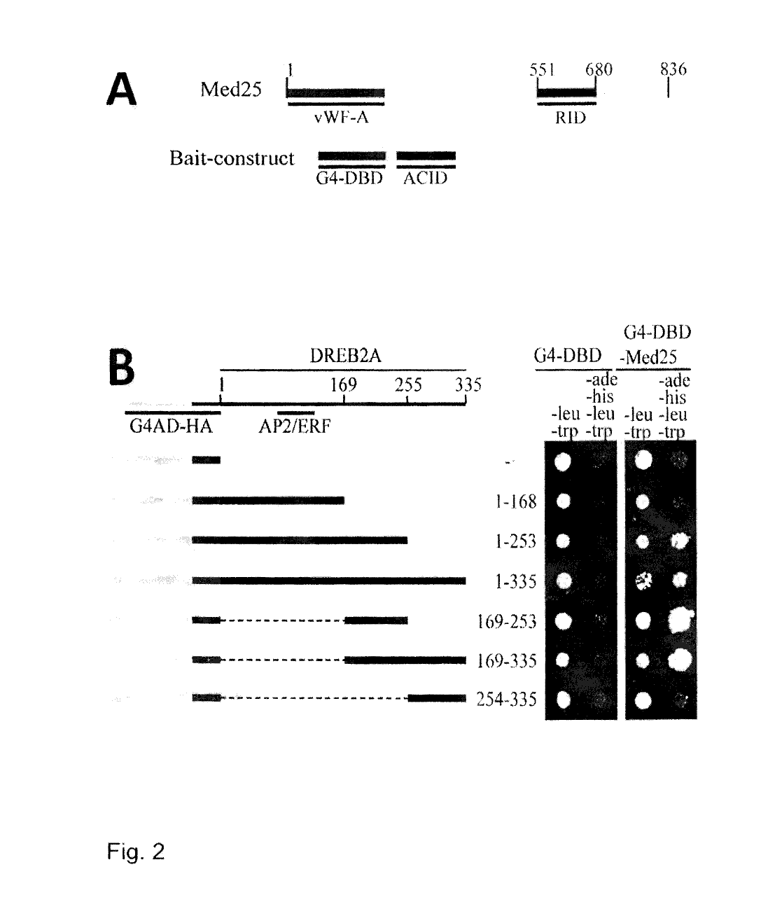

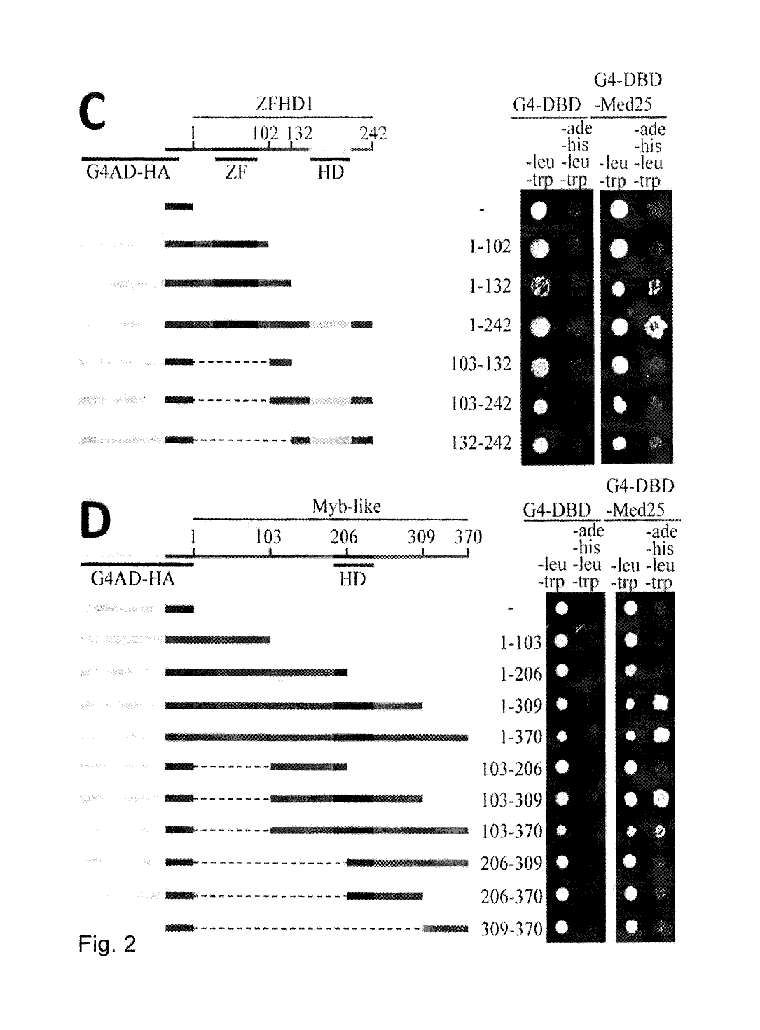

FIG. 2. Identification of regions of the DREB2A, ZFHD1 and MYB-like proteins that are required for interaction with the conserved domain of the A. thaliana Med25 protein.

(A) Schematic overview of A. thaliana Med25 and the Med25 bait construct used for the 2-hybrid screen: The locations of the regulator interaction domain (RID), the Mediator-binding von Willebrand factor A domain (vWF-A) and the Ga14 DNA-binding domains (G4-DBD) are indicated.

(B-D) 2-Hybrid interactions: The DREB2A (B), ZFHD1 (C) and MYB-like derivatives (D) used are shown. The GAL4 activation domains (G4-AD), the HA epitope tag (HA), the DNA-binding domain of DREB2A (AP2-ERF), the zinc-finger dimerization domain of ZFHD1 (ZF) and the DNA binding homeodomains of ZFHD1 and MYB-like (HD) are indicated. Growth on plates without tryptophan and leucine shows the presence of both the bait and prey 2-hybrid plasmids. Growth on plates without adenine and histidine, in addition, indicates the expression of the two reporter genes. The panels to the right illustrate growth in the absence (left) and presence (right) of Med25 (Bait construct).

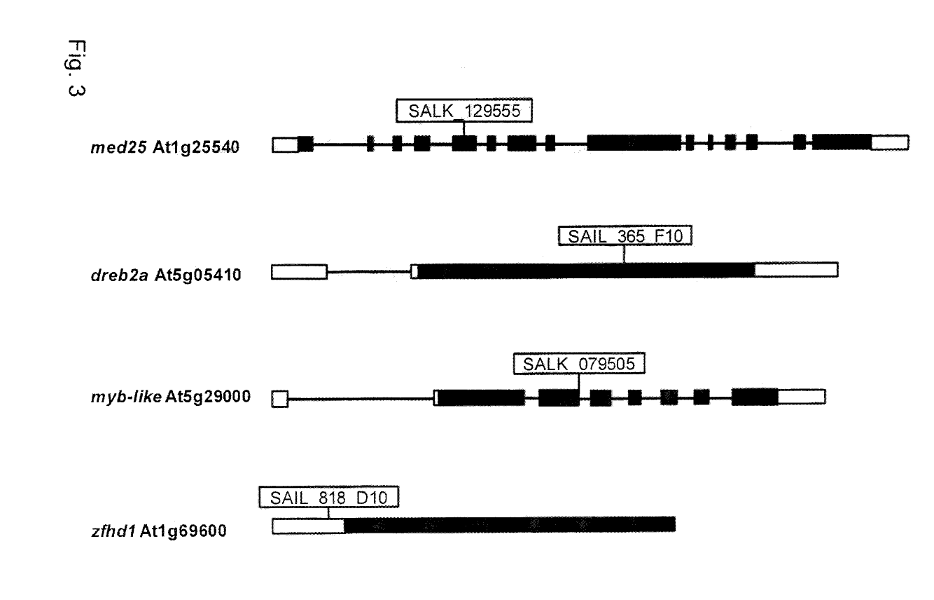

FIG. 3. Schematic representation of the MED25, DREB2A, MYB-LIKE and ZFHD1 genes.

Schematic representation of the MED25, DREB2A, MYB-LIKE and ZFHD1 genes and the location of T-DNA insertions in med25 (At1 g25540, SALK_{—}129555), dreb2a (At5g05410, SAIL_{—}365_F10), myb-like (At5g29000, SALK_{—}079505) and zfhd1 (At1g69600, SAIL_{—}818_D10), respectively. Coding regions (black boxes), untranslated regions (grey boxes), promoter regions (white boxes) and introns (solid black lines) are indicated.

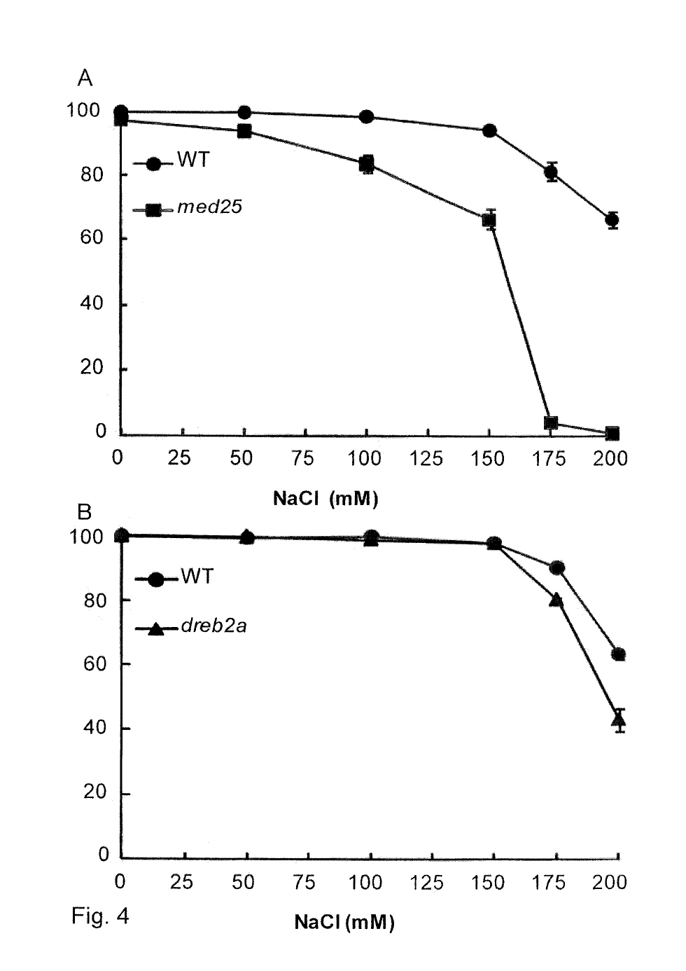

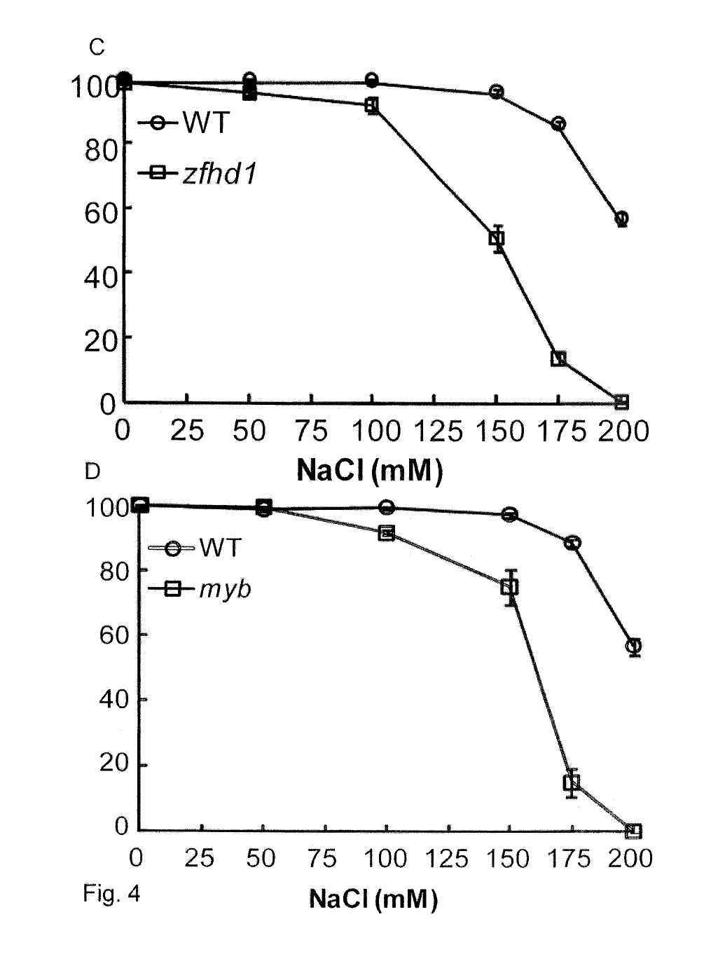

FIG. 4. Response of the Arabidopsis thaliana med25, dreb2a, zfhd1 and myb-like mutants to salt stress.

Seeds of med25, dreb2a, zfhd1 and myb-like A. thaliana mutants were incubated at 4° C. for 1 day on ½ MS solid medium with different concentrations of NaCl, and then placed at 23° C. for 5 days after which germination was scored. Each mutant was treated independently: (A) med25, (B) dreb2a, (C) zfhd1, (D) myb-like. The experiments were performed using 4 plates of 49 seedlings for each treatment and genotype. Data represent mean±standard deviation of at least 3 individual experiments.

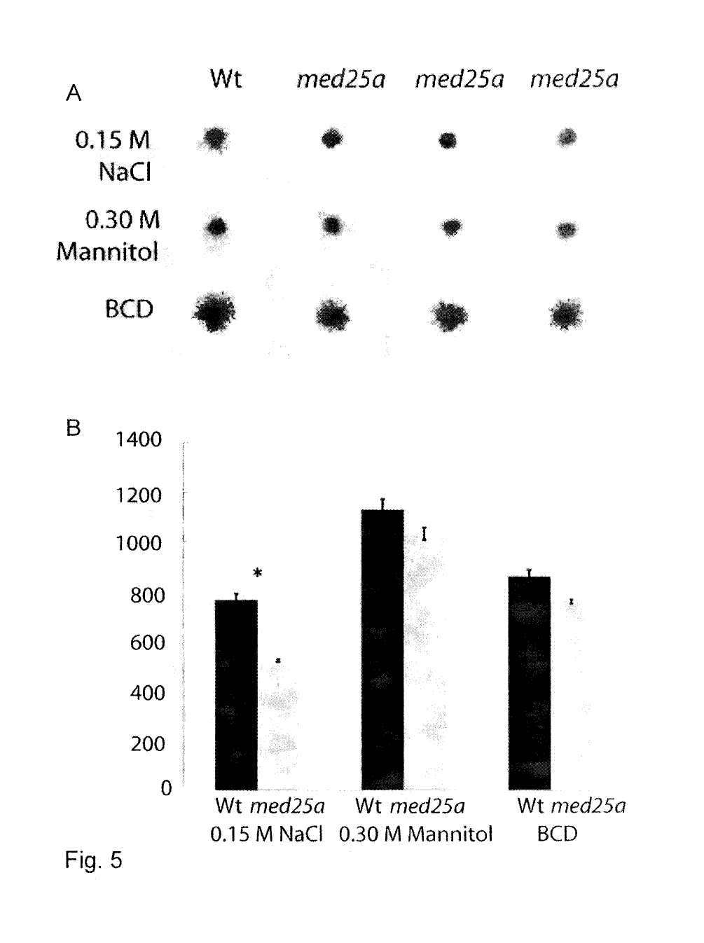

FIG. 5. Effect of salt on colony growth in Physcomitrella wild type and med25a knock-out strains.

A. Representative pictures of colonies from each of the 3 med25a knockout strains and the wild type control after 21 days growth in normal light intensity (30 μmol/m2s). The upper row shows growth on BCD (1 mm MgSO4, 1.85 mm KH2PO4, 10 mm KNO3, 45 μm FeSO4, 1 mm CaCl2, 1× Hoagland's number 2 solution, and 0.8% agar with 0.15 M NaCl. The middle row shows BCD with 0.30 M mannitol as osmotic control and the bottom row just BCD, (where BCD media comprises 1 mM MgSO₄, 1.85 mM KH₂PO₄, 10 mM KNO₃, 45 μM FeSO₄, 1 mM CaCl₂, 1×Hoagland's Number 2 solution). B. Average colony diameter of wild type and med25a mutant strains under different conditions. The mean values from 4 colonies (WT) or 12 colonies (med25a)±S.E.M. are shown. The significances of the observed differences were tested using a two-tailed, two-sampled t-test assuming unequal variances. The star denotes a difference that is significant at p=0.0014.

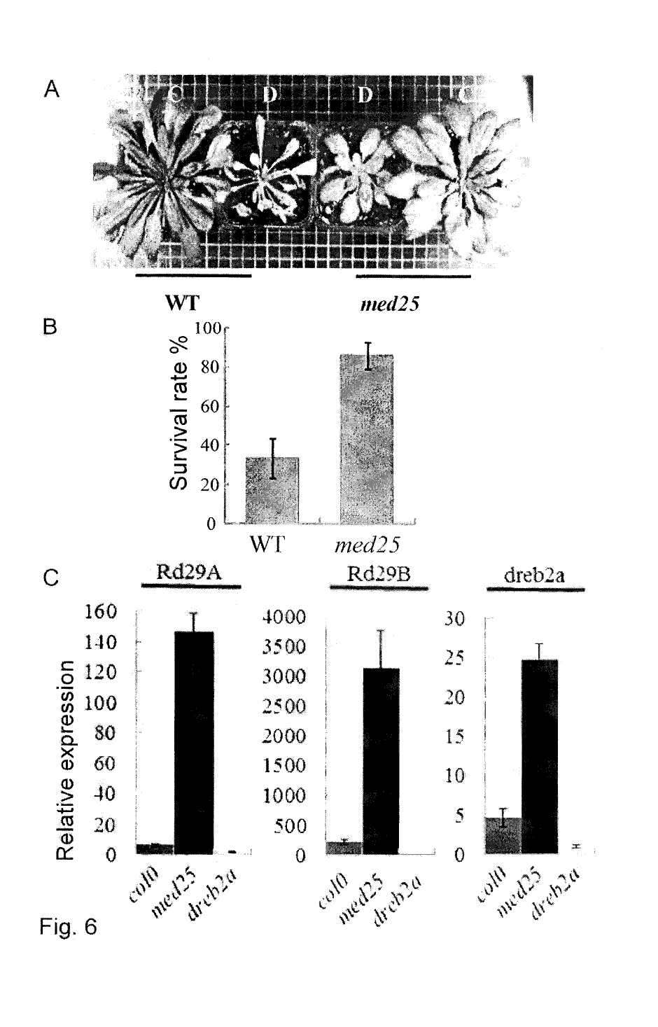

FIG. 6. Drought resistance of the Arabidopsis thaliana med25 mutant versus wild type grown under short day illumination.

(A) Phenotypes of wild type and med25 mutant plants after drought stress in short day conditions (9 h/15 h; light/dark) at 22° C. under white light fluorescent tubes (40-70 μmol·m⁻²·s⁻¹). Plants were grown on soil mixed with vermiculite (2:1) with normal watering conditions for 4 weeks, and then split in two groups. One group (D, Drought) was grown without watering for 3 weeks in the same light condition and then re-watered once. The other group (C, Control) was grown for 4 weeks in the same light under normal watering conditions. (B) Survival of plants after drought stress was assessed 7 days after re-watering. The experiment was performed using 15 plants for each genotype and treatment. Data represent mean±standard deviation of 3 individual experiments.

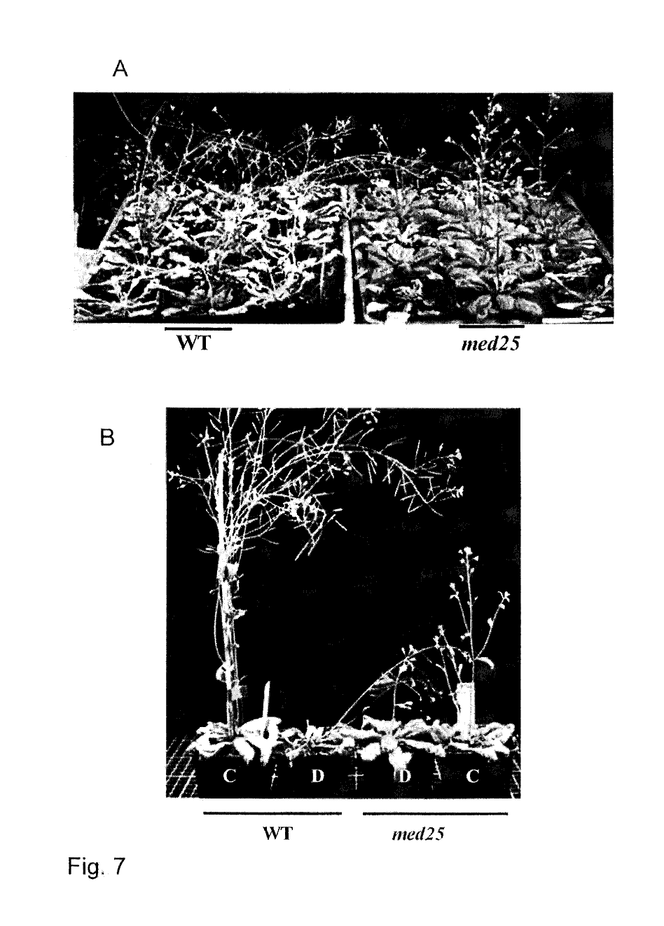

FIG. 7. Drought resistance of the Arabidopsis thaliana med25 mutant versus wild type grown under long day illumination.

(LD). Plants were grown on soil mixed with vermiculite (2:1) with normal watering conditions for 3 weeks for LD (16 h/8 h; light/dark) at 22° C., under white light fluorescent tubes (40-70 μmol·m⁻²·s⁻¹). Then, one part was grown for 3 additional weeks in the same light condition but without watering (D, Drought) and re-watered once. The other part of plants (C, Control) was grown in the same light and watering conditions. A. Pictures of one experiment of drought stress in LD conditions 7 days after re-watering. B. Picture of the most representative plants for each treatment and genotype 7 days after re-watering in LD conditions.

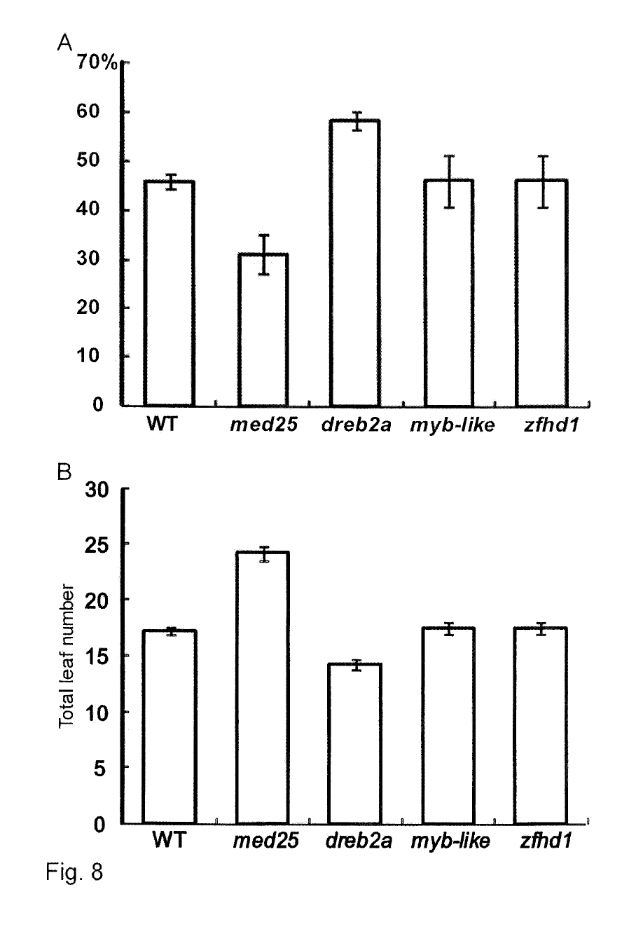

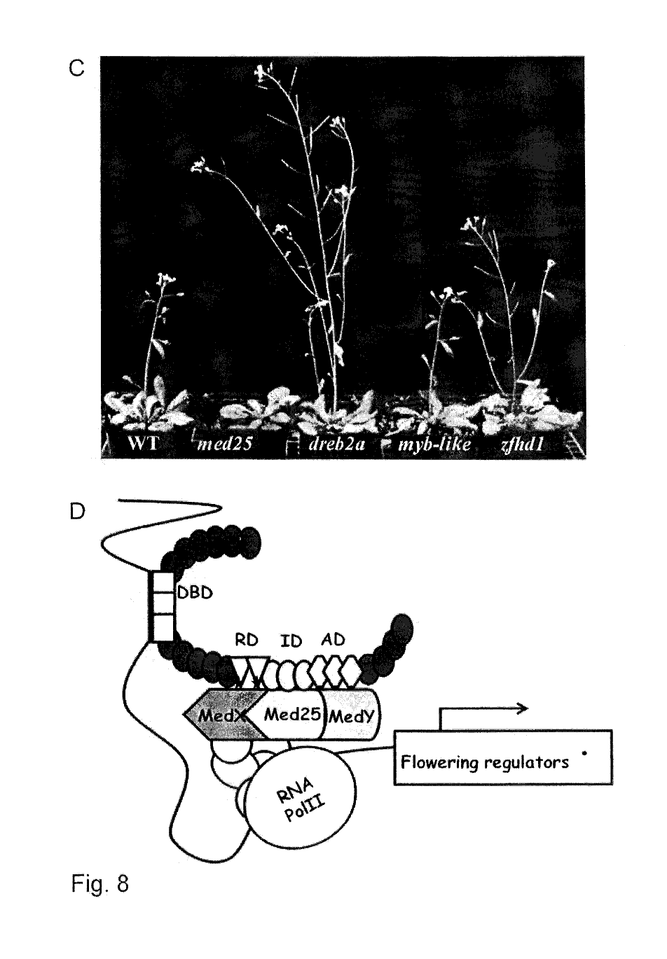

FIG. 8. DREB2A functions downstream of PhyB in a light quality pathway and has an opposite effect on flowering time compared to Med25.

(A) Hypocotyl length of 6 d old seedlings of the indicated genotypes grown for 5 days under 10 μmol m-2 s-1 of red light. (B) Flowering time of the different genotypes grown under long-day conditions (16 h light/8 h dark; 22° C./16° C.). The experiments in (A) and (B) were performed using 5 plates of at least 20 seedlings for each treatment and genotype. Data represent mean±standard deviation of at least 3 individual experiments. (C) Effects on hypocotyl lengths under normal light conditions. Wild type and mutant plants described in A and B were cultivated in long-day conditions for 4 weeks. (D) A model for how the DREB2A-Mediator interactions regulate flowering time in response to light quality. The DNA-binding domain (DBD), repressing domain (RD), Med25 interaction domain (ID), and activation domain (AD) of DREB2A are indicated. MedX and MedY represent two so far unidentified Mediator subunits.

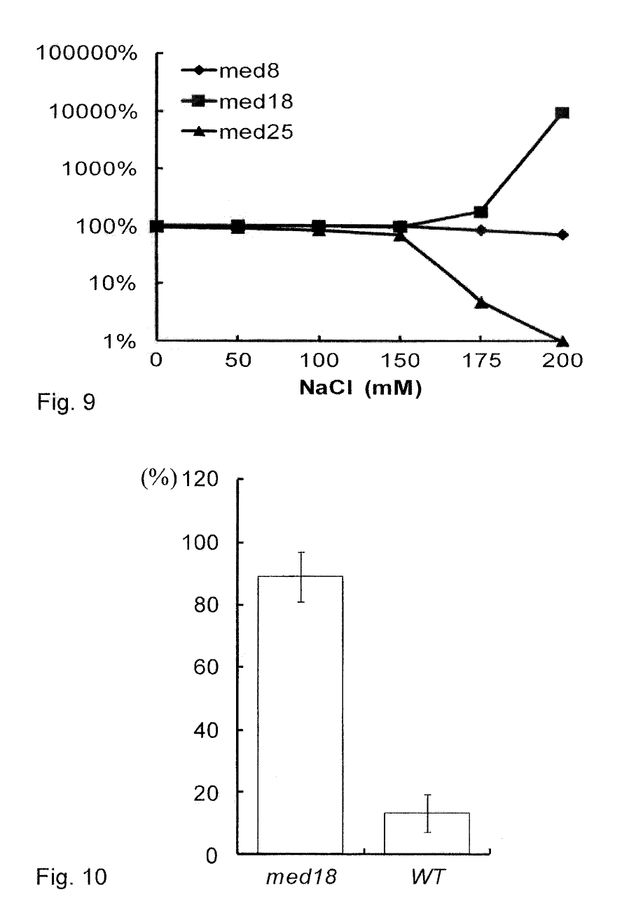

FIG. 9. Resistance of med8, med18 and med25 Arabidopsis thaliana T-DNA mutants to salt stress. Seeds of the indicated mutants were incubated at 4° C. for 1 day on ½ MS solid medium with different concentrations of NaCl, then placed at 23° C. for 5 days after which germination was scored. Each genotype was treated independently. The experiments were performed using 4 plates of 49 seedlings for each treatment and genotype. Data represent mean±standard deviation of at least 3 individual experiments. Survival is given as % Survival relative to wild type plants.

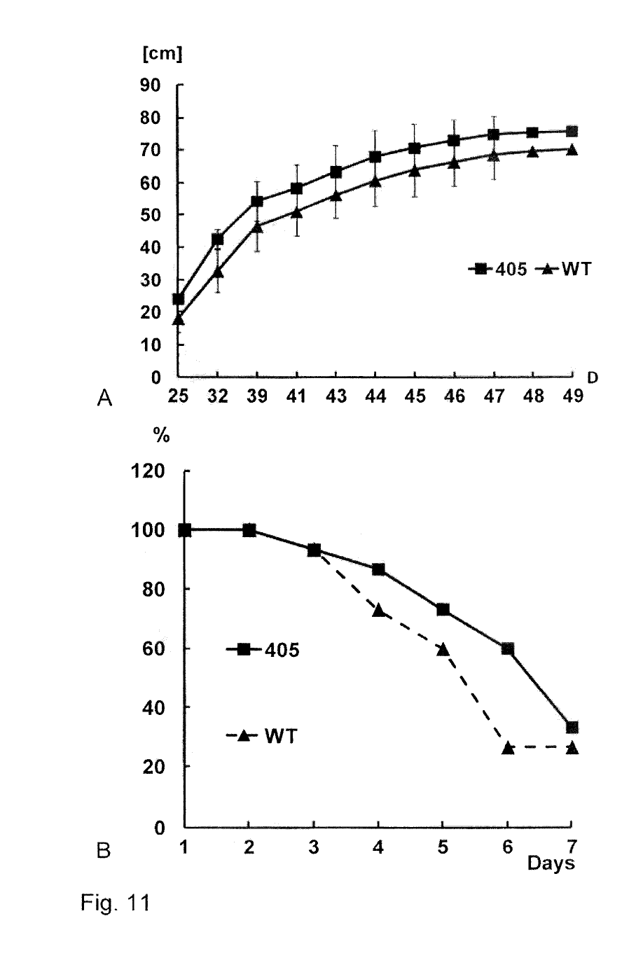

FIG. 10. Resistance of med18 Arabidopsis thaliana T-DNA mutants to drought stress. Fifteen Med18-deficient mutant plants and fifteen wild type plants were grown under normal watering conditions for 4 weeks, and then the plants divided two groups. One group was grown for 3 weeks without watering under constant illumination conditions and then re-watered once and the survival rate assessed 7 days after re-watering. The control group was grown for 4 weeks under the same illumination conditions and normal watering conditions. Data represents mean±standard deviation of 3 individual experiments.

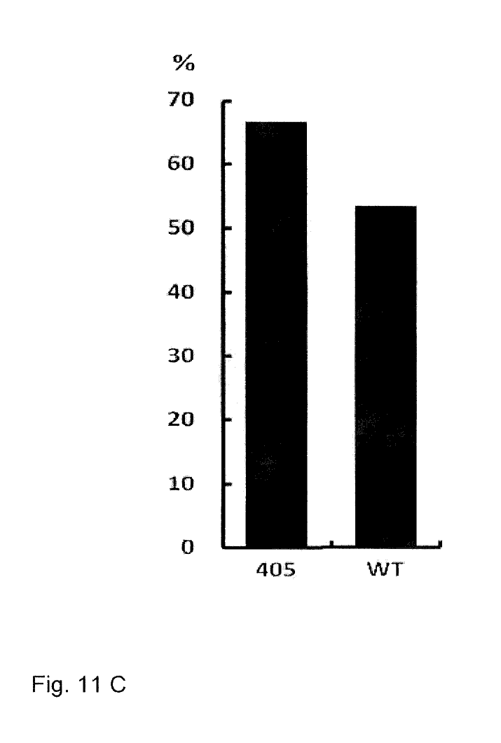

FIG. 11. Resistance of a transgenic Poplar comprising a MED18 construction group to exposure to drought stress

(A) Growth rate of the wild type (WT) and MED18 transgenic construction group 405 before and after the drought stress. The arrow indicates the time point when drought stress was initiated;

(B) The percentage of growing trees during the drought stress.

(C) Survival rate of poplar trees in each construction group after drought stress.

Trees were transferred to soil and grown under long day illumination conditions (18 h, 22° C./6 h, 15° C.; light/dark). After 6 weeks, the trees were grown without watering for 7 day, and subsequently watered where the survival rate was scored after 4 days. The size of trees was scored weekly before the drought stress and daily during the drought stress period. The experiment was performed using 3 trees for each line, 5 lines belonging to the construction group 405 and a wild type tree 15 WT (Clone T89).

### I. MEDIATOR SUBUNITS ACT AS A HUB IN TRANSCRIPTIONAL REGULATION

### I.I Med25

The polypeptide Med25 is one of a core of protein subunits that make up the Mediator coactivator complex found in plants and is widely conserved through evolution in eukaryotes. Med25 is now shown to function as a hub that integrates signals from several different environmental cues to control development. The transcription factors Dreb2A, ZFHD1 and MYB-like are all shown to function as transcriptional regulators by interacting with Med25 to regulate target genes that encode proteins involved in plant response to salt stress and drought tolerance. Surprisingly, plants in which the amount or activity of Med25 is reduced or deleted are found to exhibit increased tolerance and/or resistance to water deficit.

### I.II Med18

The polypeptide Med18 is another subunit in the core of proteins that make up the Mediator coactivator complex found in plants, and whose sequence is also widely conserved through evolution in eukaryotes (FIG. 9). Surprisingly, plants in which the amount or activity of Med18 is reduced or deleted are found to exhibit increased tolerance and/or resistance to both water deficit and salt stress.

### II STRUCTURAL AND FUNCTIONAL DOMAINS OF MEDIATOR SUBUNITS

### II.I Med25

Med25 is a subunit of the Mediator coactivator complex, found in eukaryotes, which conveys signals from promoter-bound regulatory transcription factors to the pol II/GTFs, required for the control of gene transcription. Med25 is a polypeptide, with a molecular mass of about 80-120 kDa, and is characterised by a conserved “vWF-A-like” domain in the N-terminal half of the polypeptide, corresponding to the core Mediator-binding von Willebrand factor domain (vWF-A) in humans; and a conserved activator-interacting (ACID) domain (also called regulator interaction domain RID) localized in the C-terminal half of the polypeptide. The amino acid sequences of these two functional domains of Med25 are conserved in plants (see Table 1):

The conserved amino acid sequence of the “vWF-A-like” domain in plants is:

(E/D)(G/S/T)TAA(L/M/I)GP(Y/F)VVXXXXX(D/E)Y(L/V/I)
(D/E)(K/E)(I/M)(V/I)R(S/C/Y)(6-29X)(E/D)(L/F)(S/A)
(L/I)VX(FN)(H/N)XHGX(Y/L)(S/C)(A/G/S)XXVQR(S/T)(G/
A)WT(K/R)DX(D/S/N)XF(L/F/I)XWLX(G/A/S)(I/L/M)XFXGG
G(F/L)X(D/E)(A/V)(A/S)(I/T)XEGL(A/S)EAL(K/M)(M/I)
(L/F)(15-17X)(H/N)C(L/I/V)L(V/I)(A/T)A(S/N/T)N P(Y/
H)XLXTPV(Y/F)(21-23X)AEX(V/L)AXXFXXXX(V/I)SLS(V/I)
(V/I)(S/C)PKQLP(T/K)(L/I)(K/R)X(I/L)(Y/F)(N/T)(A/S)
(G/A)K(R/P)NX(Q/R)XXD(P/L)X(V/L/I)(D/E).

Four domains (A1 to A4) within the “vWF-A-like” domain, specified below, have the most highly conserved amino acid sequences:

SEQ ID NO: 1;
vWF-A1: (E/D)(G/S/T)TAA(L/M/I)GP(Y/F)VVXXXXX(D/E)Y
(L/V/I)(D/E)(K/E)(I/M)(V/I)R(S/C/Y) having
SEQ ID NO: 2;
vWF-A2: (E/D)(L/F)(S/A)(L/I)VX(FN)(H/N)XHGX(Y/L)(S/
C)(A/G/S)XXVQR(S/T)(G/A)WT(K/R)DX(D/S/N)XF(L/F/I)XW
LX(G/A/S)(I/L/M)XFXGGG(F/L)X(D/E)(A/V)(A/S)(I/T)XEG
L(A/S)EAL(K/M)(M/I)(L/F) having
SEQ ID NO: 3;
vWF-A3 (H/N)C(L/I/V)L(V/I)(A/T)A(S/N/T)NP(Y/H)XLXTP
V(Y/F) having
SEQ ID NO: 4;
vWF-A4: AEX(V/L)AXXFXXXX(V/I)SLS(V/I)(V/I)(S/C)PKQL
P(T/K)(L/I)(K/R)X(I/L)(Y/F)(N/T)(A/S)(G/A)K(R/P)NX
(Q/R)XXD(P/L)X(V/L/I)(D/E) having

wherein X is any amino acid, selected from alanine, aspartic acid, asparagine, arginine, cysteine, glutamic acid, glutamine, glycine, histidine, isoleucine, leucine, lysine, methionine, phenylalanine, proline, serine, threonine, tryptophan, tyrosine, and valine.

Preferably, peptide (A1) and peptide (A2) are linked by a peptide having a length of between 6 and 29 amino acid residues.

Preferably, peptide (A2) and peptide (A3) are linked by a peptide having a length of between 15 and 17 amino acid residues.

Preferably, peptide (A3) and peptide (A4) are linked by a peptide having a length of between 19 and 21 amino acid residues. Where two or more amino acids are given as alternatives at a given position, if one of these amino acids is given in bold font, this indicates that it is the most highly conserved amino acid at this position.

The conserved amino acid sequence of the “ACID domain” in plants comprises 3 peptide sequences localised in sequential sequence of (a), (b) and (c) in the C-terminal half of Med25:

[SEQ ID NO: 5]
Peptide (a): KY(V/I)KXWEGXLSGQRQGQPV(F/L/I)IX(K/R)
(L/M)E(G/A)(Y/F)
[SEQ ID NO: 6]
Peptide (b):
LA(A/S)XWPXXMQIVRLI(S/A)Q(D/E)HMNNKQYVGKADFLVFR(T/
A)(M/L)(N/S)XHGFLXQLQ(E/D)KKL
[SEQ ID NO: 7],
Peptide (c):
CAVIQLPSQTLLLS(V/M)(S/A)DKAXRLIGMLFPGDMVVFKPQ

wherein X is any amino acid, selected from alanine, aspartic acid, asparagine, arginine, cysteine, glutamic acid, glutamine, glycine, histidine, isoleucine, leucine, lysine, methionine, phenylalanine, proline, serine, threonine, tryptophan, tyrosine, and valine.

Preferably, peptide (a) and peptide (b) are linked by a peptide having a length of between 8 and 14 amino acid residues.

Preferably, peptide (b) and peptide (c) are linked by a peptide having a length of between 0 and 35 amino acid residues. Where two or more amino acids are given as alternatives at a given position, if one of these amino acids is given in bold font, this indicates that it is the most highly conserved amino acid at this position.

Some amino acid residues in the amino acid sequence of the Med25 polypeptide or peptides thereof show conservative substitutions, e.g. within the group of basic amino acids (arginine, lysine and histidine), acidic amino acids (glutamic acid and aspartic acid), polar amino acids (glutamine and asparagine), hydrophobic amino acids (leucine, isoleucine, valine and methionine), aromatic amino acids (phenylalanine, tryptophan and tyrosine), and small amino acids (glycine, alanine, serine and threonine). Conservative amino acid substitutions do not generally alter the functional properties of a polypeptide, and the most commonly occurring substitutions are between Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Accordingly, a Med25 polypeptide of the present invention, comprises two domains, a “vWF-A-like” domain and an “ACID domain” whose respective functions are thought to facilitate binding to the mediator complex and to facilitate interaction with transcription factors, whereby Med25 acts as a hub to control the tolerance and/or resistance to water deficit and/or salt stress resistance in a plant or a plant cell. The “vWF-A-like” domain and the “ACID” domain are peptide regions within the Med25 polypeptide, wherein the “vWF-A-like” domain peptide comprises 4 peptides, having amino acid sequences [SEQ ID NO: 1, 2, 3, and 4], in consecutive order starting from the most N-terminal peptide, and the “ACID” domain comprises 3 peptides, having amino acid sequences [SEQ ID NO: 5, 6, and 7], in consecutive order starting from the most N-terminal peptide.

In a preferred embodiment, the Med25 polypeptide, comprises:

a “vWF-A-like” domain comprising 4 peptides, having amino acid sequences [SEQ ID NO: 1, 2, 3, and 4], in consecutive order, wherein peptide (A1) [SEQ ID NO: 1] and peptide (A2) [SEQ ID NO: 2] are linked by a peptide having a length of between 6 and 29 amino acid residues; peptide (A2) [SEQ ID NO: 2] and peptide (A3) [SEQ ID NO: 3] are linked by a peptide having a length of between 15 and 17 amino acid residues and peptide (A3) [SEQ ID NO: 3] and peptide (A4) [SEQ ID NO: 4] are linked by a peptide having a length of between 19 and 21 amino acid residues; and
an “ACID” domain comprises 3 peptides, having amino acid sequences [SEQ ID NO: 5, 6, and 7], in consecutive order, wherein peptide (a) [SEQ ID NO: 5] and peptide (b) [SEQ ID NO: 6] are linked by a peptide having a length of between 8 and 14 amino acid residues; peptide (b) [SEQ ID NO: 6] and peptide (c) [SEQ ID NO: 7] are linked by a peptide having a length of between 0 and 35 amino acid residues. The Med25 polypeptide of the present invention preferably has a molecular mass of about 80 to about 120 KDa.

In a preferred embodiment, the Med25 polypeptide of the invention is a polypeptide comprising both a “vWF-A-like” domain comprises 4 peptides, having amino acid sequences [SEQ ID NO: 2, 3, 4 and 5], and the “ACID” domain comprising three peptides having [SEQ ID NO: 6, 7 and 8], and wherein the amino acid sequence of the “vWF-A-like” domain and each peptide of the “ACID” domain of Med25 polypeptide share, respectively, at least 58%, 60%, 65%, 70%, 75%, 80%, 85%, 90%, 91%, 92%, 93%, 94%, 95%, 96%, 97%, 98%, 99%, 99.5%, or greater amino acid residue sequence identity to the corresponding domain of a Med25 polypeptide of Vitis vinifera having SEQ ID NO: 9., when compared and aligned for maximum correspondence, as measured using one of the following sequence comparison algorithms or by visual inspection.

The term “percent sequence identity” indicates a quantitative measure of the degree of homology between two amino acid sequences of equal length. When the two sequences to be compared are not of equal length, they are aligned to give the best possible fit, by allowing the insertion of gaps or, alternatively, truncation at the ends of the polypeptide sequences or nucleotide sequences. The (Nref-Ndlf)l00 can be calculated as <Nref>, wherein Nd[iota]f is the total number of non-identical residues in the two sequences when aligned and wherein Nref is the number of residues in one of the sequences. The percent sequence identity between one or more sequence may also be based on alignments using the clustalW software (http://www.ebi.ac.uk/clustalW/index. html).

An example of a Med25 polypeptide, comprising both a “vWF-A-like” domain comprises 4 peptides, having amino acid sequences [SEQ ID NO: 1, 2, 3, and 4], in consecutive order starting from the most N-terminal peptide, and an “ACID” domain comprising three peptides, having amino acid sequences [SEQ ID NO: 5, 6, and 7], in consecutive order starting from the most N-terminal peptide, and wherein the amino acid sequence of the “vWF-A-like” domain and the “ACID” domain of the Med25 polypeptide share, respectively, at least 58% and 80% amino acid sequence identity, respectively to the corresponding domain of Med25 polypeptide of Vitis vinifera having SEQ ID NO: 9, is a Med25 polypeptide selected from among:

Vitis vinifera (GSVIVT0101193900) [SEQ ID NO: 9]; Arabidopsis thaliana (At1g25540) [SEQ ID NO: 11]; Brachypodium distachyon (Bradi4g27750.1) [SEQ ID NO: 13]; Carica papaya (Cpa evm model supercontig 1 211) [SEQ ID NO: 15]; Cucumis sativus (Cucsa 283830) [SEQ ID NO: 17]; Eucalyptus grandis (predicted) [SEQ ID NO: 19]; Glycine max (Glyma02g10880) [SEQ ID NO: 21]; Medicago trunculata (Medtr5g068600) [SEQ ID NO: 23]; Mimulus guttatus (mgv1a001668m) [SEQ ID NO: 25]; Oryza sativa (Os09g13610) [SEQ ID NO: 27]; Populus trichocarpa (POPTR0010s13870) [SEQ ID NO: 29]; Populus2 (POPTR_{—}0008s11650) [SEQ ID NO: 31]; Sorghum bicolor (Sb02g020790) [SEQ ID NO: 33]; Triticum aestivum (EF029089) [SEQ ID NO: 35]; Zea mays (GRMZM2G138178 TO1) [SEQ ID NO: 37];

In a preferred embodiment a Med25 polypeptide has at least 70, 75, 80, 85, 90, 95 percent amino acid sequence identity to a Med25 polypeptide having an amino acid sequence selected from among SEQ ID NOs: 9, 11, 13, 15, 17, 19, 21, 23, 25, 27, 29, 31, 33, 35, and 37.

### II.II Med18

Med18 is a subunit of the Mediator coactivator complex, found in eukaryotes, and in yeast it interacts with Med20. Med18 is a polypeptide, with a molecular mass of about 20-25 kDa, and is characterised by a highly conserved amino acid sequence (see Table 2), and shares at least 70%, 75%, preferably 80% or 85%, more preferably 90%, 91%, 92%, 93%, 94%, 95%, 96%, 97%, 98%, 99%, 99.5%, or greater amino acid residue sequence identity to the amino acid sequence of Ricinus communis Med18 polypeptide having [SEQ ID NO: 65] when compared and aligned for maximum correspondence, as measured using a sequence comparison algorithm or by visual inspection as defined under II.I.

An example of a plant Med18 polypeptide is a polypeptide selected from among: Arabidopsis lyrata [SEQ ID NO: 39]; Arabidopsis thaliana [SEQ ID NO: 41]; Brachypodium distachyon [SEQ ID NO: 43]; Carica papaya [SEQ ID NO: 45]; Cucumis sativus [SEQ ID NO: 47]; Eucalyptus grandis [SEQ ID NO: 49]; Glycine max 1 [SEQ ID NO: 51]; Glycine max 2 [SEQ ID NO: 53]; Glycine max 3 [SEQ ID NO: 55; Manihot esculenta [SEQ ID NO: 57]; Mimulus guttatus [SEQ ID NO: 59]; Oryza sativa [SEQ ID NO: 61]; Populus trichocarpa [SEQ ID NO: 63]; Ricinus communis [SEQ ID NO: 65]; Sorghum bicolor [SEQ ID NO: 67]; Vitis vinifera [SEQ ID NO: 69]; and Zea mays [SEQ ID NO: 71];

In a preferred embodiment a Med18 polypeptide has at least 70, 75, 80, 85, 90, 95 percent amino acid sequence identity to a Med18 polypeptide having an amino acid sequence selected from among SEQ ID NOs: 39, 41, 43, 45, 47, 49, 51, 53, 55, 57, 59, 61, 63, 65, 67, 69 and 71.

### II.III Methods for Identifying Med18 and Med25 Proteins and Corresponding Genes

Orthologs and paralogs of a Med18 protein having SEQ ID NO: Z or Med25 protein having SEQ ID NO: 10, and their corresponding genes/cDNAs can be identified employing public BLAST resources and subsequently using T-coffee programs to align and select sequences. Implementation of such identification and selection methods is illustrated in Example 7.

### III NUCLEIC ACID MOLECULES ENCODING MEDIATOR SUBUNITS

### III.I Med25

A MED25 nucleic acid molecule of the present invention encodes a Med25 polypeptide as defined under section II.I.

A MED25 nucleic acid molecule encoding a Med25 polypeptide of the invention is, in one embodiment, a MED25 nucleic acid molecule having a nucleic acid sequence that has at least 60%, 70%, 75%, preferably 80% or 85%, more preferably 90%, 91%, 92%, 93%, 94%, 95%, 96%, 97%, 98%, 99%, 99.5%, or greater nucleic acid residue sequence identity to a MED25 nucleic acid molecule selected from among: Vitis vinifera (GSVIVT0101193900) [SEQ ID NO: 8]; Arabidopsis thaliana (At1g25540) [SEQ ID NO: 10]; Brachypodium distachyon (Bradi4g27750.1) [SEQ ID NO: 12]; Carica papaya (Cpa evm model supercontig 1 211) [SEQ ID NO: 14]; Cucumis sativus (Cucsa 283830) [SEQ ID NO: 16]; Eucalyptus grandis (predicted) [SEQ ID NO: 18]; Glycine max (Glyma02g10880) [SEQ ID NO: 20]; Medicago trunculata (Medtr5g068600) [SEQ ID NO: 22]; Mimulus guttatus (mgv1a001668m) [SEQ ID NO: 24]; Oryza sativa (0s09g13610) [SEQ ID NO: 26]; Populus trichocarpa (POPTRb0010s13870) [SEQ ID NO: 28]; Populus2 (POPTR_{—}0008s11650) [SEQ ID NO: 30]; Sorghum bicolor (Sb02g020790) [SEQ ID NO: 32]; Triticum aestivum (EF029089) [SEQ ID NO: 34]; and Zea mays (GRMZM2G138178 T01) [SEQ ID NO: 36].

### III.II Med18

A MED18 nucleic acid molecule of the present invention encodes a Med18 polypeptide having a molecular mass of about 20 to about 25 Kda., having a nucleic acid sequence that has at least 60%, 70%, 75%, preferably 80% or 85%, more preferably 90%, 91%, 92%, 93%, 94%, 95%, 96%, 97%, 98%, 99%, 99.5%, or greater nucleic acid residue sequence identity to a MED18 nucleic acid molecule selected from among: Arabidopsis lyrata [SEQ ID NO: 38]; Arabidopsis thaliana [SEQ ID NO: 40]; Brachypodium distachyon [SEQ ID NO: 42]; Carica papaya [SEQ ID NO: 44]; Cucumis sativus [SEQ ID NO: 46]; Eucalyptus grandis [SEQ ID NO: 48]; Glycine max 1 [SEQ ID NO: 50]; Glycine max 2 [SEQ ID NO: 52]; Glycine max 3 [SEQ ID NO: 54]; Manihot esculenta [SEQ ID NO: 56]; Mimulus guttatus [SEQ ID NO: 58]; Oryza sativa [SEQ ID NO: 60]; Populus trichocarpa [SEQ ID NO: 62]; Ricinus communis [SEQ ID NO: 64]; Sorghum bicolor [SEQ ID NO: 66]; Vitis vinifera [SEQ ID NO: 68]; and Zea mays [SEQ ID NO: 70].

### IV METHODS FOR PRODUCING A GENETICALLY MODIFIED PLANT OF THE INVENTION THAT HAS INCREASED TOLERANCE AND/OR RESISTANCE TO WATER DEFICIT AND/OR SALT STRESS RESISTANCE

The Mediator subunits, Med25 and Med18, act as hubs to control the tolerance and/or resistance to water deficit and/or salt stress resistance in a plant or a plant cell. According to the present invention, a reduction in the functional activity of Med25 in a plant or a cell thereof, confers an increased tolerance and/or resistance to water deficit to said plant or plant cell. Similarly, a reduction in the functional activity of Med18 in a plant or a cell thereof, confers an increased tolerance and/or resistance to water deficit and salt stress to said plant or plant cell. The following methods serve to illustrate alternative means for down-regulating or silencing the functional activity of Med25 or Med18 in a plant cell, where the Med25 polypeptide or Med18 polypeptide are each encoded by a nucleic acid molecule in the genome of the plant cell.

### IV.I Induced Mutation and TILLING in the MED25 and MED18 Nucleic Acid Molecule (MED25 Gene and MED 18 Gene)

The down-regulation or silencing of expression of a MED25 or MED18 nucleic acid molecule (as defined above under section III) encoding Med25 and Med18 respectively in a plant cell can be achieved by means of mutations, such as point mutations, in the MED25 or MED18 genes. Mutations can be introduced randomly into the genome of a plant cell, and then mutagenized plant cells can be selected by specific methods such like TILLING (Targeting Induced Local Lesions IN Genomes). For the TILLING, mutations are induced by treatment of individual samples of plant tissue (e.g. seeds or other regenerable tissue) with a chemical mutagen (for example EMS). Genomic DNA is then prepared from these individuals and arrayed in pools for initial screening. These pools become templates for PCR using primers that amplify a region of the MED25 or the MED18 nucleic acid molecule. For this purpose a series of primers can be prepared whose sequence are complementary to a region of the upper or lower strand of the MED25 or MED18 nucleic acid molecule, where the primers serve to screen the length of the MED25 or MED18 genes. Heteroduplexes are formed between wild type and mutant fragments in the pool by denaturing and re-annealing PCR products. These heteroduplexes are the substrate for cleavage by the nuclease CEL I. After digestion, the resulting products are visualized using standard fluorescent sequencing slab gel electrophoresis. Positive pools are then re-screened as individual DNAs, thus identifying the mutant plant and the approximate position of the mutation along the sequence. This positional information increases the efficiency of sequence analysis, as heterozygous mutations may be otherwise difficult to identify. High-throughput TILLING is for example described in Colbert et al. (2001) Plant Physiology 126: 480-484 and has recently been applied to crops [reviewed in Slade and Knauf, Transgenic Res. 2005 April; 14(2): 109-15]. Selected regenerated plants carrying (non-silent) silencing mutations in the MED25 or MED18 genes are then screened for the expression of the Med25 or Med18, and for an increased tolerance and/or resistance to water deficit as the result of reduced expression of functional Med25 or Med18 in the plant or plant cell thereof. Plants and plant cells, in which expression of a MED25 or MED18 gene is down-regulated or silenced as the result of a chemically induced mutation in their genome, are to be considered to be “genetically modified”, and since they do not comprise a transgene introduced into their genome they are not considered to be recombinant plants or plant cells.

### IV.II ECO-TILLING in the MED25 or MED18 Nucleic Acid Molecule (MED25 Gene or MED18 Gene)

Down-regulation or silencing of expression of either a MED25 or a MED18 nucleic acid molecule (as defined above under section III) encoding Med25 and Med18 respectively in a plant cell can also result from natural mutations occurring in natural plant populations, that result in (non-silent) silencing mutations in the MED25 or MED18 genes. Eco-tilling employs the TILLING method to identify these natural occurring mutations (polymorphisms) in plant populations as opposed to screening for mutations experimentally induced in a plant. The primers and methods employed in ECOTILLING are the same as those described for TILLING described above (section IV.I).

### IV.III T-DNA Insertion in the MED25 or MED 18 Nucleic Acid Molecule (MED25 Gene or MED18 Gene)

Down-regulation or silencing of expression of either a MED25 or a MED18 nucleic acid molecule (as defined above under section III) encoding Med25 and Med18 respectively in a plant cell can also be obtained by T-DNA mutagenesis [Koncz et al. (1992) Plant Mol. Biol. 20(5): 963-976], whereby the T-DNA is used to randomly introduce mutations in the plant genome followed by selecting plants comprising (non-silent) silencing mutations in the endogenous MED25 or MED18 genes. The plant, or plant cell, in which either the endogenous MED25 or MED18 gene is mutated can later be identified by PCR or other high throughput technologies using a series of PCR primer pairs spanning the MED25 or the MED18 gene [Krysan et al., (1999) T-DNA as an insertional mutagen in Arabidopsis, Plant Cell, 11, 2283-2290].

### IV.IV Directed Mutagenesis in the MED25 or MED 18 Nucleic Acid Molecule (MED25 Gene or MED18 Gene)

Vectors expressing an untranslatable form of a gene, e.g., sequences comprising one or more stop codons, or nonsense mutation, can also be used to down-regulate or silence the expression of either a MED25 or MED18 nucleic acid molecule (as defined above under section III) encoding Med25 and Med18 respectively in a plant cell. Methods for producing such constructs are described in U.S. Pat. No. 5,583,021. In particular, such constructs can be made by introducing a premature stop codon into the gene. One way of performing targeted DNA insertion is by use of the retrovirus DNA integration machinery as described in WO2006078431. This technology is based on the possibility of altering the integration site specificity of retroviruses and retrotransposons integrase by operatively coupling the integrase to a DNA-binding protein (tethering protein). Engineering of the integrase is preferably carried out on the nucleic acid level, via modification of the wild type coding sequence of the integrase by PCR. The integrase complex may thus be directed to a desired portion of genomic DNA, within the MED25 or MED18 genes, thereby producing a (non-silent) silencing mutation into the MED25 or MED18 gene.

### IV.V Antisense Transgenes for Silencing Med25 or Med18 Expression

Down-regulating or silencing expression of either a naturally occurring MED25 or MED18 gene in a host plant can be obtained by transforming a transgene comprising a nucleic acid molecule (as defined above under section III) encoding a Med25 or Med18 polypeptide or a part thereof, or a molecule whose nucleic acid sequence is the anti-sense sequence of a nucleic acid molecule encoding a Med25 or a Med18 polypeptide or a part thereof, into the host plant. Varieties of traditional sense and antisense technologies are known in the art, e.

g., as set forth in Lichtenstein and Nellen (1997), Antisense Technology: A Practical Approach IRL Press at Oxford University, Oxford, England. The objective of the antisense approach is to use a sequence complementary to the target gene to block its expression and create a mutant cell line or organism in which the level of a single chosen protein is selectively reduced or abolished. For antisense suppression, a nucleic acid molecule (as defined above under section III; e.g. cDNA) encoding Med25 or Med18, or part thereof, is arranged in reverse orientation (i.e. antisense with respect to the coding sequence) relative to a nucleic acid molecule comprising a promoter sequence comprised within the transgene. The transgene, when stably introduced into the genome of a plant cell, need not correspond to the full length MED25 or MED18 cDNA or gene, and need not be identical to the MED25 or MED18 cDNA or gene found in the plant type to be transformed. The antisense sequence of the nucleic acid molecule need only be capable of hybridizing to the gene or RNA encoding Med25 or Med18. Thus, where the transgene comprises an antisense nucleic acid molecule that is of shorter length, a higher degree of nucleic acid sequence identity [preferably at least 50, 60, 70, 80, 85, 90, 95 or 100% nucleic acid sequence identity] to the endogenous sequence encoding Med25 or Med18 will be needed for effective antisense suppression. While antisense nucleic acid molecules of various lengths can be utilized, preferably, the introduced antisense in the transgene will range from 15-30 nucleotides in length, such as from 16-28 nucleotides, from 17-26 nucleotides or from 18-24 nucleotides in length, and improved antisense suppression will typically be observed as the length of the antisense increases. Preferably, the length of the antisense will be greater than 100 nucleotides. Transcription of an antisense nucleic acid molecule, as described, results in the production of RNA molecules that are the reverse complement of mRNA molecules transcribed from the endogenous MED25 or MED18 gene in the plant cell. For more elaborate descriptions of anti-sense regulation of gene expression as applied in plant cells reference is made to U.S. Pat. No. 5,107,065, the content of which is incorporated herein in its entirety.

### IV.VI RNAi Transgenes for Silencing Med25 or Med18 Expression

Down-regulating or silencing expression of a naturally occurring MED25 or MED18 gene in a host plant can be obtained by “RNA interference” or “RNAi”: RNAi employs a double-stranded RNA molecule or a short hairpin RNA to change the expression of a nucleic acid sequence with which they share substantial or total homology. The term “RNAi down-regulation” refers to the reduction in the expression of a nucleic acid sequence mediated by one or more RNAi species. The term “RNAi species” refers to a distinct RNA sequence that elicits RNAi. In plants, however, the gene silencing caused by RNAi can spread from cell to cell in plants, and the effects of RNA interference are thus both systemic and heritable in plants. For further details of RNAi gene suppression in plants by transcription of a dsRNA, reference is made to U.S. Pat. No. 6,506,559, US Patent Application Publication No. 2002/0168707 Al, and U.S. patent application Ser. No. 09/423,143 (see WO 98/53083), Ser No. 09/127,735 (see WO 99/53050) and Ser. No. 09/084,942 (see WO 99/61631), all of which are incorporated herein by reference in their entirety.

Suppression of the MED25 or MED18 gene by RNA interference can be achieved using a transgene comprising a nucleic acid molecule functioning as a promoter that is operably linked to a nucleic acid molecule comprising a sense and anti-sense element of a segment of genomic DNA or cDNA of the MED25 or MED18 gene (comprising a nucleic acid molecule as defined above under section III), e.g., a segment of at least about 17 nucleotides, such as at least 25, at least 30, at least 40, at least 50, at least 75, at least 100, at least 200, at least 300, at least 400, at least 500, or at least 750 nucleotides, or such as at least 1 kb, such as at least 1.5 kb, at least 2 kb, at least 2.5 kb, or such as at least 3 kb, where the sense and anti-sense DNA components can be directly linked or joined by an intron or artificial DNA segment that can form a loop when the transcribed RNA hybridizes to form a hairpin structure. A fragment of at least 17 nucleotides of said transcribed ribonucleic acid molecule has at least 50, 60, 70, 80, 85, 90, 95 or 100% nucleic acid sequence identity to a nucleic acid molecule encoding a Med25 polypeptide or a Med18 polypeptide. The promoter can be selected from a constitutive, inducible, or tissue specific promoter that is operably 5-prime linked to said nucleic acid molecule comprising a sense and anti-sense element. Such a nucleic acid molecule has been described by Brummel D. A. et al., Plant Journal 2003, 33, pages 10 793-800).

In another example, an artificial microRNA is constructed were a promoter drives the expression of an RNA molecule mimicking the function of a microRNA and the sequence setting the gene specificity is recombinantly introduced (Niu et al, 2006. Science 2006, vol 24:1420-1428) The microRNA can be of natural occurrence and only overexpressed.

In a particular embodiment of the present invention the nucleic acid construct, or recombinant DNA construct, further comprises a strong constitutive promoter in front of a transcribed cassette consisting of part of the target gene followed by a plant functional intron followed by the same part of the target gene in reverse orientation. The transcribed cassette is followed by a terminator sequence. The preferred vector is of such type with one of the nucleotide sequence of the invention is inserted in inverted repeat orientation.

The presently preferred nucleic acid construct for RNAi based approaches is a vector termed 25 pK7GWIWG2(I). The vector is described in Gateway vectors for Agrobacterium-mediated plants transformation, Karimi, M. et al., Trends In plant Sciences, Vol 7 no 5 pp 193-195. The same basic kind of vector were earlier described in Wesley S. V. et al., Construct design for efficient, effective and high-throughput gene silencing in plants. Plant Journal 2001, 27, pages 581-590.

A person skilled in the art will understand that any sequence being part of the MED25 or MED18 gene, or the corresponding mRNA's presented here can be used to down regulate the levels of such mRNA. In the case the presented sequence does not represent the full mRNA, the full mRNA can be cloned with various techniques known to a person skilled in the arts, such as the techniques described in Sambrook et al. A recent resource important for finding more sequences associated with the mRNA transcripts of Populus genes is the published genome of Populus tricocarpa and the resources described in Tuskan et al 2006 (G. A Tuskan et al, 2006. The genome of Black Cottonwood, Populus tricocarpa (Torr. & Gray). Science vol 313 No. 5793, pages 1596-1604.

### IV.VII Construction of Vectors for Silencing Med25 or Med18 Expression

In general, those skilled in the art are well able to construct vectors of the present invention and design protocols for recombinant gene expression. For further details on general protocols for preparation of vectors reference is made to: Molecular Cloning: a Laboratory Manual-2nd edition, Sambrook et al, 1989, Cold Spring Harbor Laboratory Press.

### V A GENETICALLY MODIFIED PLANT CELL, PLANT OR PART THEREOF ACCORDING TO THE INVENTION THAT HAS INCREASED TOLERANCE AND/OR RESISTANCE TO WATER DEFICIT AND/OR SALT STRESS RESISTANCE

A genetically modified, or transgenic, plant according to the present invention characterized by increased tolerance and/or resistance to water deficit and/or salt stress resistance may be an annual plant or a perennial plant. Preferably the annual or perennial plant is a crop plant having agronomic importance, and accordingly plants that are not crop plants and are of no agronomic value (non-crop plants (weeds) such as Arabidopsis spp., are not encompassed by the present invention). The annual crop plant can be a monocot plant selected from Avena spp (Avena sativa); Oryza spp., (e.g. Oryza sativa; Oryza bicolour); Hordeum spp., (Hordeum vulgare); Triticum spp., (e.g. Triticum aestivum); Secale spp., (Secale cereale); Brachypodium spp., (e.g. Brachypodium distachyon); Zea spp (e.g. Zea mays).; or a dicot plant selected form Cucumis spp., (e.g. Cucumis sativus); Glycine spp., (e.g. Glycine max); Medicago spp., (e.g. Medicago trunculata); Mimulus spp; Brassica spp (e.g. Brassica rapa; Brassica napus; Brassica oleraceae); Beta vulgaris.

Preferably the perennial plant is a woody plant or a woody species. The woody plant may be a hardwood plant e.g. selected from the group consisting of acacia, eucalyptus, hornbeam, beech, mahogany, walnut, oak, ash, willow, hickory, birch, chestnut, poplar, alder, maple, sycamore, ginkgo, a palm tree and sweet gum. Hardwood plants from the Salicaceae family, such as willow, poplar and aspen including variants thereof, are of particular interest, as these two groups include fast-growing species of tree or woody shrub which are grown specifically to provide timber and bio-fuel for heating.

In further embodiments, the woody plant is a conifer which may be selected from the group consisting of cypress, Douglas fir, fir, sequoia, hemlock, cedar, juniper, larch, pine, redwood, spruce and yew.

In other embodiments, the woody plant is a fruit bearing plant which may be selected from the group consisting of apple, plum, pear, banana, orange, kiwi, lemon, cherry, grapevine, papaya, peanut, and fig.

Alternatively, the woody plants which may be selected from the group consisting of cotton, bamboo and rubber plants. The present invention extends to any plant cell of the above genetically modified, or transgenic plants obtained by the methods described herein, and to all plant parts, including harvestable parts of a plant, seeds and propagules thereof, and plant explant or plant tissue. The present invention also encompasses a plant, a part thereof, a plant cell or a plant progeny comprising a DNA construct according to the invention. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced in the parent by the methods according to the invention.

It should be noted that embodiments and features described in the context of one of the aspects of the present invention also apply to the other aspects of the invention. Thus, definitions of one embodiment regard mutatis mutandis to all other embodiments comprising or relating to the one embodiment. When for example definitions are made regarding DNA constructs or sequences, such definitions also apply with respect to methods for producing a plant, vectors, plant cells, plants comprising the DNA construct and vice versa. A DNA construct described in relation to a plant also regards all other embodiments.

### VI WATER DEFICIT AND/OR SALT STRESS TOLERANCE/RESISTANCE PROPERTIES OF THE GENETICALLY MODIFIED PLANT OF THE INVENTION

As used herein “water deficit” means a period when water available to a plant is not replenished at the rate at which it is consumed by the plant. A long period of water deficit is colloquially called drought. Lack of rain or irrigation may not produce immediate water stress if there is an available reservoir of ground water for the growth rate of plants. Plants grown in dry soil, however, are likely to suffer adverse effects with minimal periods of water deficit. Severe water stress can cause wilt and plant death; moderate drought can cause reduced yield, stunted growth or retarded development. Water stress tolerance requires comparison to control plants. For instance, plants of this invention can survive water deficit with a higher yield than control plants. In the laboratory and in field trials drought can be simulated by giving plants of this invention and control plants less water than an optimally-watered control plant and measuring differences in traits. In general, a control plant is a plant of the same line or variety as the genetically modified or transgenic plant being tested, lacking the specific trait-conferring, recombinant DNA that characterizes the genetically modified or transgenic plant. A suitable control plant may be the parental line used to generate the genetically modified or transgenic plant herein. A control plant may in some cases be a transgenic plant line that includes an empty vector or marker gene, but does not contain the recombinant polynucleotide of the present invention that is expressed in the transgenic plant being evaluated.

The salt concentration of water that irrigates soil can usefully be expressed as parts per million of the dissolved salts w/w in the water. Fresh water typically has less than 1,000 ppm salt; slightly saline water typically has from 1,000 ppm to 3,000 ppm; moderately saline water typically has from 3,000 ppm to 10,000 ppm; highly saline water typically has from 10,000 ppm to 35,000 ppm; while ocean water typically has 35,000 ppm of salt. Plants tolerant to slightly saline to moderately saline soil are advantageous.

### VII BREEDING GENETICALLY MODIFIED PLANTS HAVING WATER DEFICIT AND/OR SALT STRESS TOLERANCE/RESISTANCE PROPERTIES

Any genetically modified or transformed plant obtained according to the invention can be used in a conventional breeding scheme or in in vitro plant propagation to produce more genetically modified or transformed plants with the same characteristics and/or can be used to introduce the same characteristic in other varieties of the same or related species. In this manner, the genetically modified genes or transgenes conferring water deficit and/or salt stress tolerance/resistance can be transferred to an elite (commercial relevant) crop variety by for example (marker assisted) crossing, Furthermore, the plants of the present invention can be further improved with stacked traits, e.g., a genetically modified or transformed plant having water deficit and/or salt stress tolerance/resistance properties according to the invention, can be stacked with other traits of agronomic interest.

### EXAMPLES

### Example 1

### Stress-Related Transcription Factors Interact with the Conserved ACId Domain of Arabidopsis thaliana Med25

### 1.1 The 2-Hybrid Screening Method

This method was used to screen and identify plant transcriptional regulators that operate through interaction with the ACID domain in the Arabidopsis thaliana Med25. The bait was composed of amino acids 551-680 of Arabidopsis Med25, the region corresponding to the VP16-interaction domain in the human Med25 (see FIG. 3B in ref. 6). The bait was used with a prey comprising a cDNA library generated from inflorescence meristem, floral meristem and floral buds in the two-hybrid screen.

The Yeast Two-Hybrid screen was performed according to the instructions of the Matchmaker Two-Hybrid System 3 (CLONTECH). The bait was constructed by PCR amplification of the nucleotide sequence 1651-2040 of the open reading frame (ORF) of the A. thaliana Med25 (At1g25540) encoding amino acids 551-680 of Med25 using the Arabidopsis cDNA library CD4-16 as template and the primers:

AtMed25-EcoRI-aa551-fwd (5′-GGG GAC AAG TTT GTA CAA AAA AGC AGG CTc cga att cAC TTC ACA ATC CAA ATA TGT GAA-3′) [SEQ ID NO: 72] and AtMed25-SalI-aa680-rev (5′-GGG GAC CAC TTT GTA CAA GAA AGC TGG GTg gtc gac tta ATT TGG AAT TTG TGG TTT AAA CA-3′) [SEQ ID NO: 73]. The PCR product was cloned into the Ga14 DNA binding domain (BD) vector pGBKT7 by digestion of both plasmid and vector with EcoRI and SalI (Fermentas, Burlington, Ontario, Canada) and purification using Jetquick PCR purification kit (Genomed, Gmbh, Löhme, Germany). Ligation of the digested plasmid and PCR product were performed with T4 DNA Ligase (Invitrogen,) according to the manual, transformed into TOP10 cells and selected for kanamycin resistance on LB agar plates (25 μg kanamycin/ml). Plasmids from resulting clones were analysed by DNA sequencing. The plasmid was transformed into the yeast strain AH109 (MATa, trp1-901, leu2-3, 112, ura3-52, h is 3-200, gal4Δ, ga180Δ, LYS2::GAL1UAS-GAL1TATA-HIS3, GAL2UAS-GAL2TATAADE2, URA3::MEL1 UAS-MEL1TATA-lacZ, MEL1) by using the lithium acetate method as described in the Clontech manual.

The prey comprised a cDNA library (CD4-30) cloned into the Ga14 Activation Domain plasmid pAD-GAL4-2.1. The CD4-30 library (see http://www.arabidopsis.org/abrc/catalog/cdna_library_{—}1.html) and the cDNA library CD4-16 (11) were obtained from the Arabidopsis Biological Resource Center (ABRC). The Escherichia coli strain TOP10 (F-mcrA Δ(mrr-hsdRMSmcrBC) φ80lacZΔM15 ΔlacX74 nupG recA1 araD139 Δ(ara-leu)7697 galE15 galK16 rpsL(StrR) endA1 λ−) (Invitrogen, Carlsbad, Calif., USA) was used for cloning in bacteria.

Yeast AH109 transformed with pGBKT7-Med25₅₅₁₋₆₈₀ or empty pGBKT7 were both able to grow on SD/-Trp/-His plates due to leakiness of the HIS3 promoter. However, the self-activation/leakage of the HIS3 reporter gene was completely suppressed by addition of 0.5 mM 3-Amino-1,2,4-triazole. Growth was also completely suppressed by growing the cells on SD/-Trp/-Ade/-His media. Equal expression from the bait plasmids was confirmed by western blotting using monoclonal anti-myc primary antibodies.

A fresh colony of AH109 containing the bait plasmid pGBKT7-Med25₅₅₁₋₆₈₀ was inoculated into 50 ml of SD/-Trp and incubated at 30° C. overnight. The culture was inoculated into 1.7 liters of 2×YPDA medium and incubated at 30° C. with shaking until OD600 ˜0.6. Cells from the culture were pelleted and made competent, transformed with 2 mg cDNA library in plasmid pAD-GAL4-2.1, and plated according to the instructions for library scale transformation in the Matchmaker GAL4 Two-Hybrid System 3 User Manual (Clontech). The transformation mix was spread on 80 large (140 mm) plates containing 60 ml of SD/-Ade/-His/-Leu/-Trp (QDO) for high stringency selection. Diluted fractions of the transformation mix were spread on six plates containing SD/-Leu/-Trp for estimation of transformation efficiency. After growth for 14-16 days, the yeast colonies appearing on QDO were re-plated on YPD and single colonies were re-plated on QDO medium prior to plasmid isolation.

### 1.2 Isolation and Identification of Positive Prey Plasmids

Approximately 2.5×10⁶ cDNA clones were screened. pAD-GAL4-2.1-cDNA plasmids (from the cDNA library CD4-30) were isolated from colonies growing on QDO with the lyticase method described in the Matchmaker GAL4 Two-Hybrid System 3 User Manual (Clontech), and transformed into TOP10 (Invitrogen) cells. Transformants were plated on LB-agar supplemented with carbenicillin (100 μg/ml). Subsequently, the pAD-GAL4-2.1-cDNA constructs isolated from these TOP10 clones were re-transformed into AH109 cells and sequentially transformed with pGBKT7-Med25₅₅₁₋₆₈₀ or empty pGBKT7 and plated on QDO media to evaluate positive clones. Positive prey plasmids were sequenced and used to search GenBank using BLAST (http://www.ncbi.nlm.nih.gov/blast). All positive cDNA prey clones originated from one of the three different genes, At1g69600, At5g29000, and At5g05410 (FIG. 1), indicating that the screen was saturated.

1.3 Characterization of cDNA Encoded Proteins Interacting with the ACID Domain in the Arabidopsis thaliana Med25

The 3 types of 2-hybrid positive clones identified encoded the transcription factors: DREB2A (At5g05410), ZFHD1 (At1g69600) and MYB-like (At5g29000). None of these transcription factors had previously been associated with light quality pathways. Rather, DREB2A belongs to a protein family that also includes DREB1A-C and DREB2B. They bind to the dehydration-response element/C-repeat (DRE/CRT) motif which is involved in drought and cold stress response (6). Overexpression of full length DREB2A does not result in activation of downstream genes. However, overexpression of DREB2A lacking a repressing domain (RD: see below) results in a growth retardation phenotype and rounded, slightly darker leaves with short petioles (7). ZFHD1 belongs to a family of proteins that binds to the promoter region of the EARLY RESPONSIVE TO DEHYDRATION STRESS 1 (ERD1) gene and causes up-regulation of several stress-inducible genes as well as a considerable increase in drought tolerance (8). Finally, the MYB-like protein has not been studied in detail but it was identified in a transcriptome analysis as one of 454 transcripts that are specifically expressed in plants subjected to a combination of drought and heat stress (9).

### Example 2

### Transcriptional Activation Domains of Dreb2A, ZFHD1 and Myb-Like Interacting with the Conserved ACID Domain of Arabidopsis thaliana Med25

The region within each transcription factor required for interaction with the Med25₅₅₁₋₆₈₀ region (FIG. 2A) was identified using the 2-hybrid assay. DREB2A amino acids 169-254 was shown to be the minimal domain required for interaction with Med25₅₅₁₋₆₈₀ (FIG. 2B). Since this domain neither overlaps with the previously identified TAD in DREB2A, which comprises amino acids 254-335, nor with the RD located between amino acids 136-165 (7), it is probable that these domains have separate functions. However, the DREB2A region between amino acids 1-169, which includes the RD, has a negative effect on the interaction between DREB2A and Med25₅₅₁₋₆₈₀. By contrast, a good correlation was seen between the previously identified TAD in ZFHD1 (amino acids 1-102) (8) and the minimal region for interaction with Med25₅₅₁₋₆₈₀ (amino acids 1-132) (FIG. 2C). The Med25₅₅₁₋₆₈₀ interaction domain in Myb-like was localized to the region between amino acids 103-309 (FIG. 2D). No TAD has previously been reported for this protein, but the Myb-like DNA-binding domain (DBD) is located between amino acids 184-248, which overlaps with a homeodomain-like region located between amino acids 227-291.

### Example 3

### Arabidopsis Thaliana Mutants Lacking Dreb2A, ZFHD1, Myb-Like or Med25 are Sensitive to Salt Stress

Arabidopsis mutants in the Columbia accession, obtained from the Arabidopsis

Biological Resource Center (ABRC), have T-DNA insertions in the genes that encode DREB2A, ZFHD1, and MYB-like as well as the MED25/PFT1 gene (FIG. 3). The seed stock numbers N629555 (med25), N873547 (dreb2a), N579505 (myb-like) and N877090 (zfhd1) were obtained from the Nottingham Arabidopsis Stock Center (NASC). All mutants were identified after screening of the Salk T-DNA insertion lines (12). Homozygous plants of the different mutants were identified using the primer sequences given at http://signal.salk.edu/tdnaprimers.2.html and used in the experiments described here.

When seeds of these mutant plants were set to germinate at different NaCl concentrations, all of the mutants had a reduced percentage of germination compared to the wild type, consistent with an increased sensitivity to salt stress (FIG. 4). The sensitivity to salt stress of med25 mutant was at least as pronounced as for the transcription factor mutants, consistent with the interaction between the transcription factors and Med25 shown by the 2-hybrid data. This newly observed phenotype of med25 strongly supports the notion that Med25 functions downstream of DREB2A, ZFHD1, and MYB-like. The zfhd1, myb-like and med25 mutants show higher sensitivity to NaCl than the dreb2a mutant.

### Example 4

### The Function of Med25 in Salt Stress Resistance is Conserved Among Land Plants

All embryophytes (land plants) have physiological systems for dealing with drought and salt stress. The role of Med25 in stress resistance is shown to be conserved during plant evolution, by demonstrating the effect of deleting this gene in the moss Physcomitrella patens. The key role of Med25, in regulating drought tolerance in plant forms as divergent as moss and Arabidopsis thaliana, is very strong evidence that the Med25 protein is responsible for regulating drought tolerance in all members of the plant kingdom.

### 4.1 Targeted Gene Disruption in Physcomitrella.

Deletion of the single gene, PpMED25A, encoding an intact Med25 protein P. patens was performed by gene targeting (10). The Physcomitrella genome contains two AtMED25-related sequences: PpMED25A (Phypa1_{—}1:170131) encodes an intact Med25 protein, while PpMED25B (Phypa1_{—}1:92911) is an apparent pseudogene, which has two frameshifts followed by stop codons in exon 7, and a deletion of 2104 by that starts near the end of exon 7 and ends in intron 10. This deletion removes sequences corresponding to codons 253-559 of PpMED25A and creates a third frameshift. The PpMED25A gene was PCR amplified from genomic DNA and cloned into the EcoRI site of pRS426 plasmid. A selection cassette containing the hpt marker was then inserted between the two BglII sites in PpMED25A, resulting in the deletion of codons 43-838 (of 878). The targeting construct was released from the vector by Swal digestion, and then transformed into moss protoplasts (10), where stable transformants were then selected in the presence of 30 mg/l hygromycin B (Sigma H3274).

### 4.2 A Med25 Knockout Mutant of Physcomitrella Exhibits Salt Sensitivity

The selected Physcomitrella med25a knockout mutants have an increased sensitivity to salt, shown by a 32% reduction in the colony diameter in the presence of 0.15M NaCl as compared to the wild type (FIG. 5). No effect was seen in the presence of an osmotic control (0.3M mannitol). The role of Med25 in salt stress resistance is thus an ancient function that was present already in an early embryophyte.

### Example 5

### An Arabidopsis thaliana Mutant Lacking Med25 is Resistant to Drought

The drought tolerance of the med25 mutant was tested under short day growth conditions where flowering is inhibited, in order to avoid that the delayed flowering phenotype of this mutant (ref. 10) indirectly affects its sensitivity to drought. Surprisingly, we found that the med25 mutant is drought resistant compared to wild type plants (86.2% survival compared to 33.3% for wild type plants) (FIG. 6). Furthermore, the Med25 mutant showed the same phenotype in long day growth conditions (FIG. 7). Over-expression of ZFHD1 or of a constitutively active form of DREB2A leads to drought resistance (14, 15). Thus, Med25 has an opposite function compared to ZFHD1 and DREB2A in regulating a plant's response to drought.

qRT-PCR was used to study the drought induced rd29a and rd29b mRNAs in wild type and med25 and dreb2A mutants (7). Both rd29a and rd29b mRNAs were strongly up-regulated in response to drought in the med25 mutant (150 to 3200 fold) and severely down-regulated in response to drought in the dreb2A mutant (FIG. 6C). In addition Dreb2A mRNA was strongly up-regulated in response to drought in the med25 mutant. Accordingly, the drought response phenotype is correlated with the expression of these stress-induced genes.

### Example 6

### The Arabidopsis thaliana Dreb2a Protein is Involved in Light Quality Pathways that Control Flowering Time

The three transcriptional regulators, DREB2A, ZFHD1 and Myb-like, shown to interact with Med25 had previously only been implicated in the responses to different types of stress. However, Med25 itself, was originally identified as PFT1 acting as a downstream effector in the PhyB pathway which regulates light quality-controlled flowering time. The hypocotyl length response and leaf number (which is a measure of flowering time) for each of the mutants revealed that the myb-like and zfhd1 mutants are identical to the wild type, while the phenotype of the dreb2a mutant is the opposite to the med25 mutant (FIG. 7). Since dreb2a has an early flowering phenotype, comparable to that of phyB, this indicates that DREB2A could function in the phyB pathway. This is a previously unidentified and unexpected function for the DREB2A transcription factor and supports the theory that the PhyB acts indirectly on Med25 (PFT1), through DNA-bound transcription regulatory factors.

### Example 7

### Transgenic Populus Spp Trees Silenced for Med18 or Med25 Expression

### 7.1 Methods for Identifying Orthologs and Selecting Corresponding Genes

BLAST resources on Phytozome, the tool for green plant comparative genomics (JGI—The Joint Genome Institute and CIG—Center for Integrative Genomics) were used to identify homologous sequences of the Arabidopsis thaliana Med18 and Med25 genes. The amino acid sequence of the Arabidopsis Med18 and Med25 polypeptides were blasted against the genome sequence of Black Cottonwood, Populus trichocarpa (JGI—The Joint Genome Institute and Tuskan, et. al. Science 15 Sep. 2006: Vol. 313. no. 5793, pp. 1596-1604) using the TBLASTN algorithm. Populus trichocarpa gene sequences encoding proteins showing homology to the Arabidopsis mediator proteins were reblasted, using BLASTN and TBLASTX algorithm, to evaluate if more genes homologous to the mediator genes were present in Populus Trichocarpa. Clustal X ver. 2.0.12 (Larkin et al. (2007). Bioinformatics, 23, 2947-2948) was used for multiple alignments and for generation of phylogenetic trees of the identified sequences. These clustering methods in combination with bootstrapping analysis identify the genes having the most similar genetic characteristics and evolutionary relationships. Tools in Vector NTI Advance® software suite (Invitrogen™) were used for alignments, assemblies and modifications for evaluation of the sequences. For a person skilled in the art these methods can, in combination, be used to identify orthologous genes in other plants.

BLAST resources in Populus DB EST database (Sterky, et. al., Proc Natl Acad Sci USA. 2004 Sep. 21; 101(38):13951-6) were used to identify selected ortholog genes in Hybrid aspen, Populus tremula×P. tremuloides. The identified EST sequences were assembled, aligned and evaluated by the use of tools in Vector NTI Advance® software suite (Invitrogen™). For a person skilled in the art these methods can, in combination, be used to identify orthologous proteins, and the expressed gene sequences (e.g. cDNAs) and genes encoding these proteins in other plants.

### 7.2 Med18 Orthologs in Populus Spp.

A BLAST search of the Populus trichocarpa genome using A. thaliana Med18 sequence, AT2G22370, as query, resulted in one single gene model, POPTR_{—}0007s05200. POPTR_{—0007}s05200 has a predicted protein sequence of 217 amino acids which has 83% identity and 94% positives to AT2G22370 over 100% of the sequence, and is thus the closest ortholog in Populus trichocarpa.

A single EST (EST: A041 P22) was identified in hybrid aspen, Populus tremula×P. tremuloides, showing 99% identity over 375 by of the coding sequence of POPTR_{—}0007s05200a, and is thus the predicted ortholog for Med18 in Hybrid aspen. The sequence of EST: A041P22 was used to design primers for amplification of fragments for two separate RNAi constructs.

### 7.3 Med25 Orthologs in Populus Spp.

A BLAST search of the Populus trichocarpa genome using A. thaliana Med25 sequence, AT1G25540, as query, resulted in two gene models, POPTR_{—}0010s13870 and POPTR_{—}0008s11650, which are predicted orthologs in Populus trichocarpa. POPTR_{—}0010s13870 predicted protein sequence of 797 amino acids has 65% identity and 77% positives to AT1G25540 over 84% of the sequence, while POPTR_{—}0008s11650 predicted protein sequence of 851 amino acids has 66% identity and 78% positives to AT1G25540 over 79% of the sequence. POPTR_{—}0008s11650 and POPTR_{—}0010s13870 gene model sequences are 91% identical over more than 2 kb of coding DNA sequence and their encoded protein sequences are 89% identical over 699 amino acids. Therefore POPTR_{—}0010s13870 and POPTR_{—}0008s11650 are assumed to be paralogs in Populus trichocarpa and both of them orthologs of Arabidopsis thaliana gene AT1G25540.

The evaluation of Med25, POPTR_{—}0008s11650 and POPTR_{—}0010s13870, resulted in a set of EST sequences, showing very high homology to both of the paralogs, contained in cluster: POPLAR.8697 and singleton: C066P63. Available PopDB contig and assemblage analysis did not fully separate the paralogous sequences in Hybrid aspen. However two ESTs were selected, EST: S0 67A01 most identical to POPTR_{—}0010s13870, 98% identity over 740 by and EST: UB64CPC07 most identical to POPTR_{—}0008s11650, 98% identity over 487 bp. The sequences thereof were used to design primers for amplification of fragments for two separate RNAi constructs.

### 7.4 Cloning RNAi Constructs for Silencing MED 18 and MED25 Genes

Gateway® technology (Invitrogen™) was used for the cloning process. Gene specific primers were designed and attached with Gateway® attB recombination sites.

Gateway cloning primers for Med18 RNAi constructs:

[SEQ ID NO: 74]
KR939_F1_attB2: GGGGACCACTTTGTACAAGAAAGCTGGGTGCAAG
CAAGAATGTGCTTAGATTG
[SEQ ID NO: 75]
KR939_R1_attB1: GGGGACAAGTTTGTACAAAAAAGCAGGCTAAACA
CCTGGTTTTGACAAGTGCAG
[SEQ ID NO: 76]
KR940_F1_attB2: GGGGACCACTTTGTACAAGAAAGCTGGGTAGGGG
TTGTTCCTACTGCCG
[SEQ ID NO: 77]
KR940_R1_attB1: GGGGACAAGTTTGTACAAAAAAGCAGGCTCCCAG
CATCAAGCGGATAACTAG

Gateway cloning primers for Med25 RNAi constructs:

[SEQ ID NO: 78]
KR941_F1_attB2: GGGACCACTTTGTACAAGAAAGCTGGGTAGGTGT
CTCTTCTGGTATGAACACG
[SEQ ID NO: 79]
KR941_R1_attB1: GGGGACAAGTTTGTACAAAAAAGCAGGCTTGGTA
ACTGGATTACTGCACAAAGC
[SEQ ID NO: 80]
KR942_F1_attB2: GGGGACCACTTTGTACAAGAAAGCTGGGTGAATG
ACGTCTTCTGTGCCTGC
[SEQ ID NO: 81]
KR942_R1_attB1: GGGGACAAGTTTGTACAAAAAAGCAGGCTTGCAC
CCATTCCACTTTGTACC

The selected RNAi gene fragments, namely two Med25 RNAi [SEQ ID NO: 82 and 83] and two Med18 RNAi [SEQ ID NO: 84 and 85]) were amplified by PCR from EST cDNA clone templates and subsequently recombined into the pDONR™-201 vector (Invitrogen™) resulting in Entry clones. There after the fragments were recombined into the RNAi destination vector, pK7GWIWG2(I) (Karimi, M. et al., Trends In plant Sciences, Vol 7 no 5 pp 193-195). Insertion of the RNAi construct into a plant host will cause the constitutive expression of an inverted double stranded hairpin RNA under the control of the CaMV 35S promoter.

### 7.5 Agrobacterium-Mediated Plant Transformation to Insert RNAi Transgene

The CaMV 35S inverted repeat DNA constructs were transformed into Agrobacterium and subsequently into Hybrid aspen, Populus tremula L.×P. tremuloides Minch. Clone T89, hereafter called “poplar”, and regenerated, essentially as described in Nilsson et al. (1992) Transgenic Research 1, 209-220. Approximately 15-20 independent lines were generated for each construct. One such group of transgenic tree lines produced using one construct is hereafter called a “construction group”. Each transgenic line within each construction group, e.g. KR555-2B KR555-3A, KR555-2B and so on, are different transformation events and therefore most probably have the recombinant DNA inserted into different locations in the plant genome. This makes the different lines within one construction group partly different. For example it is known that different transformation events will produce plants with different levels of gene down-regulation when using RNAi constructs of the type used here.

The med18 or med25 gene expression level was measured by q-PCR in the independent lines for each construction group. Five lines for each construct were selected for further experimental analysis. In three lines gene expression was strongly down-regulated and in two lines gene expression was less down-regulated. One leaf of each transgenic poplar line was harvested under sterile conditions and directly frozen in liquid nitrogen. The frozen leaves were ground to a powder and 100 mg of powder was then used for total RNA extraction using the RNEasy Plant Mini Kit (Qiagen). One microgram of total RNA was used for RT-PCR of mRNA using the iScript cDNA Synthesis Kit (Bio-Rad). The resulting cDNA was used as a DNA template for amplification of specific Mediator genes by q-PCR. PCR reactions were carried out in a Light Cycler 480 (Roche) with Light Cycler 480 SYBR Green I Master (Roche Diagnostics GmbH) using the following primers for med18 lines:

[SEQ ID NO: 86],
med18POP-940 Fwd: ACTGTCCACGCTCCATGTAACAGA
[SEQ ID NO: 87],
med18POP-940 Rev: ACAAATCCACCTCATAACTCATAA
[SEQ ID NO: 88],
med18POP Fwd: AGATGCTAAAACTACATGCATTG
[SEQ ID NO: 89],
med18POP Rev: CGGTGCAAGATATTCGCAGAAAGA

and for med25 lines:

[SEQ ID NO: 90],
med25-Pt942 Fwd: AACTGTATTTTCATCTGGGCA
[SEQ ID NO: 91],
med25-Pt942 rev: CAGACCACTCATTGCGATTGG
[SEQ ID NO: 92],
med25POP Fwd: AGATGCTAAAACTACATGCCATTG
[SEQ ID NO: 93] and
med25POP Rev: AGCAATGTCTGAGATGGTAACTGG
[SEQ ID NO: 94] and
18S Fwd: CTATCAACTTTCGATGGTAGG
[SEQ ID NO: 95].
18S Rev: CCGTGTCAGGATTGGGTAATTT

18S RNA was used as an internal standard to normalize for differences in template amounts. Real time dsDNA amplification was monitored and analyzed by the Light Cycler 480 Software release 1.5.0 SP3 (Roche).

### 7.6 Growth and Phenotypic Analysis of Transgenic Trees Carrying RNAi Transgenes for Silencing MED 18 or MED25 Expression

The transgenic poplar lines were grown on soil together with their wild type control (WT) trees, in a greenhouse under long day conditions (18 hr, 22° C./6 hr, 15° C.; day/night). In a growth group a number of wild type trees (15 trees) and a number of transgenic trees comprising several construction groups (i.e. 3 trees per line and 5 lines per construction group) were grown in parallel in the greenhouse under the same above conditions. All comparisons between the wild type trees and construction groups are made within each growth group. Directed measurements, samplings and analysis are performed and the data thereof are analyzed for significant changes in for example growth increase, wood density, wood morphology, wood chemical composition, biomass production, drought stress tolerance, salt stress tolerance etc.

One construction group (with 5 different lines) for MED18 was tested for drought resistance in Poplar trees. The transgenic poplar lines were grown on soil together with the wild type control (WT) in a greenhouse in long days conditions (18 hr, 22° C./6 hr, 15° C.; light/dark). The trees were grown for 6 weeks under automatic watering conditions, and then for 7 days without watering. After this drought period, the trees were re-watered for scoring their survival rate. The growth of trees was measured weekly during the 6 first weeks then daily during the drought stress period (FIG. 11). The construction group 405 for MED18 had a taller growth phenotype than WT (FIG. 11A). Furthermore, the continued growth of group 405 trees showed them to be less sensitive to drought stress than WT trees (FIGS. 11A and B). The survival rate was scored 4 days after re-watering the trees. The 405 group had a higher survival rate (>66.6% of the trees) compared to WT trees (FIG. 11C).

### Example 8

### Arabidopsis Thaliana Mutant Lacking Med18 is Resistant to Drought and Salt Stress

Three Arabidopsis thaliana mutants having T-DNA insertions in each of MEDS, MED18 and MED25 genes that silence expression of the respective genes were compared with respect to their resistance to salt stress. Seeds of each mutant genotype were incubated at 4° C. for 1 day on ½ MS solid medium with different concentrations of NaCl, then placed at 23° C. for 5 days after which germination was scored. In contrast to med25, med18 mutants show a strong resistance to salt stress; and were even more resistant than wild type plants (FIG. 9).

Arabidopsis thaliana Med18 mutant plants are also drought tolerant as demonstrated in growing plants under water-stress conditions over a period of 4 weeks (FIG. 10), where survival of the med18 mutants was significantly enhanced over wild type plants.

### Example 9

### Arabidopsis Thaliana Mutant Lacking Med18 Show Changes in Morphology and Flowering

Med18 T-DNA mutant plants have a larger number of leaves at flowering, but they are smaller and their phyllotaxy is modified. Their leaves grow asymmetrically; and have a light green color indicating a change in chlorophyll content.

TABLE 1
Alignment of amino acid sequences of Med 25 polypeptides

| | |
|---|---|
| Arabidopsis_thaliana | MS---SEVKQLIVVAEGTAALGPYWQTIVSDYLEKIIRS----------- |
| Brachypodium_distachyon | M----AAERQLVVVVEGTAALGPYWHTIAAEYVEKIVRS----------- |
| Carica_papaya | MA-----EKKLVVIIEGTAAMGPFWKSILSDYLEEIVRSDFKLISPASLL |
| Cucumis_sativus | MA-----DKQLIVVVEGTAAMGPFWQTVVSDYIEKIVRS----------- |
| Eucalyptus_grandis | MS---TTERSLIVAVDGTAAMGPYWKTIVSDYLEKIVRS----------- |
| Glycine_max | MA-----EKQLIVAVESTAAMGPYWNTILMDYLDKMIRC----------- |
| Medicago_trunculata | MA-----EKQLIVAVETTAAMGPYWDTLLMDYLEKIVRC----------- |
| Mimulus_guttatus | MV-----PKQLIVAVEGTAAIGPFWQTIVSDYLDKIIRS----------- |
| Oryza_sativa | MAAA-AAERQLVVAVEGTAALGPYWPVTVADYVEKIVRS----------- |
| Populus2 | MA-----DKQLIVAVEGTAAMGPFWSIIVSDYLEKIIRC----------- |
| Populus_trichocarpa | MA-----EKQLIVAVEGTAAMGPFWSIIVSDYLEKIIRY----------- |
| Sorghum_bicolor | MA---AADRQLVVAVEGTAALGPYWSTIVAEYVEKIVRS----------- |
| Triticum_aestivum | MVEGMASERQLVVVVEGTAALGPYWPAIAAEYVEKIVRS----------- |
| Vitis_vinifera | MA-----EKQLVVAVEGTAAMGPYWQAVVSDYLDKIIRY----------- |
| Zea_mays | MA---AADRQLVVAVEGTAALGPYWSTIVAEYVEKIVRS----------- |
| | * :.*:* : ***:**:* :*:::::* |
| Arabidopsis_thaliana | --FCGSELNGERNPVSTVELSLVIFNSHGSYCACLVQRSGWTRDVDIFLH |
| Brachypodium_distachyon | --FCAAQLSGQKLAGVPPELALVVFHTHGPYSAFVVQRSGWTKDMDTFLS |
| Carica_papaya | RCFCGNELTGQKTSSSNVELSLVTFNCHGSYSGCLVQRSGWTKDVDTFLL |
| Cucumis_sativus | --FCGNELTGQKPPTSNVEFSLVTFHTHGSYCGCLVQRTGWTRDVDIFIQ |
| Eucalyptus_grandis | --VCA----------SNCDLSLVMFNSHGSLSACMVQRSAWTKDVSCFLE |
| Glycine_max | --FGGNDSTGQKFSASNVEFALVTYNTHGCYSSCLVQRSGWTRDPDVFFS |
| Medicago_trunculata | --LGGNESTGQKPSGSNVEFSLVTYNTHGCYSGILVQRTGWTRDPDVFLQ |
| Mimulus_guttatus | --FCGNEPTGQKPATTHFELSLVMFNAHGSYSACLVQRSGWTKDMDIFFQ |
| Oryza_sativa | --FCAHEMAGQKLAGTPPELALVVFHTHGPYSAFCVQRSGWTKDMNVFLS |
| Populus2 | --YCGNETSGQKPPTSSAELSLVTFNSHGSYSACLVQRSGWTRDVDIFLH |
| Populus_trichocarpa | -----------KVPTSIVELSIVTFNSHGSYSACLVQRSGWTRDVDIFLQ |
| Sorghum_bicolor | --FCASELPGQKLAGPPPELALVVFHTHGPYSAFDVQRSGWTKDTDAFLS |
| Triticum_aestivum | --FCSTELSGQKLAGVPPELALVVFHTHGPYSAFIVQRSGWTKDMDAFLS |
| Vitis_vinifera | --FCGNELAGQKPSSSNFELSLVMFNAHGSYCSCLVQRSGWTRDVDLFLQ |
| Zea_mays | --FCASELPGQKLVGTPPELALVVFHTHGPYSAFDVQRSGWTKDVDAFLS |
| | ::::* :: ** .. ***:.**:* . *: |
| Arabidopsis_thaliana | WLSSIQFGGGGFNEVATAEGLAEALMMFS--PPSGQAQPSNDLKRHCILI |
| Brachypodium_distachyon | WLSGISFSGGGFSEAAICEGLAEALMILQGSPSNNQNHQNHELQKHCVLV |
| Carica_papaya | WLTALPFAGGGLDDAAIAEGLAEALMMFDINLNGSQAQQNVDLQRHCILI |
| Cucumis_sativus | WLSAIPFSGGGFSDAAIAEGLAEALMMFPTQPNGGQNQQTMDMQKHCILV |
| Eucalyptus_grandis | WLSAMSFSGGGENDAAIAEGLSEALMMFPLPLNGNLPPQSLDGKRHCVLV |
| Glycine_max | WLSSIPFNGGGENDAAIAEGLSEALMMLWNSQSGAPNQQSVDMHKHCILV |
| Medicago_trunculata | WLESIPFSGGGFNDAAIAEGLAEALMMFPPSQSGGLNQQNVDTNMHCILV |
| Mimulus_guttatus | WLSAIPFAGGGENDAAIAEGLSEALMMFS-SPKGNQN-QNVEGQRHCILI |
| Oryza_sativa | WLSGISFSGGGFSEAAISEGLAEALMILQGSSSNSQNHQSHEVQKHCILV |
| Populus2 | WLSAIPFAGGGENDAAIAEGLSEALMMFPITPNGNQTQPNIDGQRNCILI |
| Populus_trichocarpa | WLSAIPFAGGGFNDAAIAEGLSEALMMFPIDPNGSQTQSNIDEQRNCILI |
| Sorghum_bicolor | WLSGISFSGGGFSEASTCEGLAEALKILQGSPNATQSHQNHEAQKHCILV |
| Triticum_aestivum | WLSGISFSGGGFSEAAICEGLAEALMILQGSSSSSQNHQNRELQKHCLLV |
| Vitis_vinifera | WLSALPFAGGGFNDAAIAEGLAEALMMFSVAANGSQTQQNVDGQRHCILV |
| Zea_mays | WLSGILFSGGGFSEASICEGLAEALKILQGNPNITQGHQNHEAQKHCILV |
| | ** .: * ***:.:.: .***:*** :: . : : :*:*: |
| Arabidopsis_thaliana | TASNPHILPTPVYRPRLQNVERNENGDAQAESRLSDAETVASYFAKCSVS |
| Brachypodium_distachyon | AASNPYPLPTPVYRPFVQSGDHKKNNEVIKESCLADAEAVAISFSQCCVS |
| Carica_papaya | AASNPYALPTPVYRPKLQNLDQNENVEGQTESRLSDAESLAKSFPMCSVS |
| Cucumis_sativus | AASNPYPLPTPVYRPAVQNLEQHDNVEP--GSSQSYAETVAKSFPQCFIS |
| Eucalyptus_grandis | AATNPYPLSTPVYRPTFQLED--EHVETYSGKPLCDAEGVAKFFPQCCVS |
| Glycine_max | AASNPYPLQTPVYVPRPQNLEQSETIDSDSGSHLYDAEAVAKAFPQFSIS |
| Medicago_trunculata | AASNPYPLQTPVYVPQLQSLEKTESIDSNQVNQLYDAEAVAKAFPQFNIS |
| Mimulus_guttatus | AASNPYPLPTPVFRPQIQNPEKNDGVESQPDNRLSDAETLAKSFAQCAVS |
| Oryza_sativa | AASNPYPLPTPVYRPLVQSSDHKENNDGAKESCLADAETVAKSFAQCSVS |
| Populus2 | AASNPHPLPTPVYRPQIQNLEQTENIDAQNESRLSDAETVAKSFPQCSVS |
| Populus_trichocarpa | AASNPHPLPTPVYRPQIQNLEQIENIDAQNESRLSDAETVAKSFPQCSVS |
| Sorghum_bicolor | AASNPYPLPTPVYCLPTQSTDHKENIETSKEPSIADAETVAKSFAQCSVS |
| Triticum_aestivum | AASNPYPLPTPVYRPSVQSSDHKKSNEATKESCLADAEAVAVSFAQCSVS |
| Vitis_vinifera | AANNPYPLPTPVYQPQMQNMEQNESIESQTESRLSDAEAVAKSFAQCSVS |
| Zea_mays | AASNPYPLPTPVYCLPTQSTDHKENIETSKEPSIADAETVAKSFAQCSVS |
| | :*.**: * ***: * : . : ** :* *. :* |
| Arabidopsis_thaliana | LSVVCPKQLPTIRALYNAGKPNQQSADLSIDTAKNIFYLVLISENFVEAC |
| Brachypodium_distachyon | LSVVSPKQLPILKAIYNAGKRNPQASDPSVDHVKNPHFLVLLSESFMEAR |
| Carica_papaya | LSVICPKQLPKLRAIYNSGKRNSRAADPQVDNVKNPQFLVLISENFMEAR |
| Cucumis_sativus | LSVICPKQLPKLKAIYTAGKRNPRAADPPIDNVKSPSYLVLISENFVEAR |
| Eucalyptus_grandis | LSVICPKQLPKLRAIYNAGKRNPRAADPPVDNGKNTHFLVLIAENFLEAR |
| Glycine_max | LSVICPKQLPKIKSIYNAGKRNNRAADPPLE-AKTPHFLILISEGFREAR |
| Medicago_trunculata | LSVVCPKQLPKIKAIYNAGKRNNRAADPPVD-AKTTHFLILISEGFREAR |
| Mimulus_guttatus | LSVICPKQLPKLRGIFNAGKRNPRAPDPPVDNNKNPHFLVLISENFMEAR |
| Oryza_sativa | LSVVSPKQLPTLKAIYNAAKRNPRAADPSVDHAKNPHFLVLLSDNFLEAR |
| Populus2 | LSVICPKQLPKLRLIYNAGKRNSRAADPPVDNIKNPHFLVLISENFMEAR |
| Populus_trichocarpa | LSIICPKQLPKLRSIYNAGKRNSRAADPPVDSVKNPHFLVLISENFMEAR |
| Sorghum_bicolor | LSVISPKQLPTLKAIYNAGKRNPRAADPSVDHAKNPHFLVLLSENFMEAR |
| Triticum_aestivum | LSVVSPKQLPTLKAIYNAGKRNPQASDPSVDHAKNPHFLVLLSESFMEAR |
| Vitis_vinifera | LSVICPKQLPKLKSIYNAGKRNPRVADPPVDNVKNPHFLVLISDSFMEAR |
| Zea_mays | LSVISPKQLPTLKAIYNAGKRNLRAADPSVDHAKNPHFLVLLSENFMEAR |
| | **::.*****.:: ::.:.* * : .* :: *.. :*:*:::.* ** |
| Arabidopsis_thaliana | AALSHSA-TNLPQTQSPVKVDRATVAPSIPVTGQPPAPVSSANGPIQNRQ |
| Brachypodium_distachyon | TALSHPSPGNLVPNQIITKMDIAPA---ATVPGPTSNANPSVNGSMMGRQ |
| Carica_papaya | AALSRPG--STLANQSPVKMDIASA TSVTGQPPASVPSVNGSVMNRQ |
| Cucumis_sativus | AALSRPGITSLPANQSPVKMDISSV---VPVTGPPPTTTPSVNGPIINRQ |
| Eucalyptus_grandis | TALSRPGTASLPSNQSPVKMDTTPP-PVNSVTAQPPASVP---------- |
| Glycine_max | GALSRSGITSLPSNQSPVKVDAVSV---TPVTGAPPTSMP-VNGSIPNRQ |
| Medicago_trunculata | SALSRPG-TNMPSNQSPVKVDAVSA---TPVTGAPPSSLPSVNGSIPNRQ |
| Mimulus_guttatus | SCLSRSGITSLPPNQIPVKMDVTPAP--PPISGPPPVSIPAVNGSLLARQ |
| Oryza_sativa | TALSRPLPGNLVTNHPITKMDTAA----TSVPVPTSNGNPSVNGPMLTRQ |
| Populus2 | AALSRPGATSLPSNQTPVKVDIASV---TSVTGPLPASIPSVNGSITNRP |
| Populus_trichocarpa | AALSLPGVTSLTSNQTPVKVDIASV---TSVTGPAPTSIPSVNGSITNRP |
| Sorghum_bicolor | TALSRPLHGNMAPNQSITKMDTAPA---VTMPGPTS------NANPSGRQ |
| Triticum_aestivum | TALSHPLPGNLVPNHTITKMDTTPA---ATVPGPPSNASPSVNGTMMGRQ |
| Vitis_vinifera | AALSRPGLSNMTANQSPVKMDIASV---PQVSAAPPASIPSVNG-IMNRP |
| Zea_mays | TALSRPLHGNLAPNQTITKMDTAPA---VTMPGPTS------NGNSSGRQ |
| | .** . . .: .*:* . :. . |
| Arabidopsis_thaliana | PVSVGPVPTATVKVEPSTVTSMAPVPSF-PHIPAVARP--ATQAIPSIQT |
| Brachypodium_distachyon | P-------TANIKVEPTIITPMVSAPAF-SHMTPISNV--TSQGVSALQS |
| Carica_papaya | PISVGNVPSTTVKVEPSTVTSMVTGPAY-PHIPSVARP--VSQGIPNLQT |
| Cucumis_sativus | PVSVPNGPTATVKVEPNTVTSMTNGSGYPPHMPSVVRA--ASQGVPSLQT |
| Eucalyptus_grandis | --------------EPTTVTSMAAGPNF-SHIPSVQRP--TTQGVSALQT |
| Glycine_max | PVPAGNVAPATVKVEPVPVTSMVSGPAF-PHNSSVPRATSTSQGVPSLQT |
| Medicago_trunculata | PIPAGNVTPATVKVEQV---PVTSGPAF-SHNPSVPRATGTGLGVPSLQT |
| Mimulus_guttatus | PVPAANIPPAIVKLEPPTIAPSMTGPTF-PHISSIPRA--VPQTVPTMQT |
| Oryza_sativa | PN----GVVANIKTEPTTLPPMVSAPAF-SHVTPVANG--VSQGLSSVQS |
| Populus2 | PISVGNLPTATVKVEPSTITSMANGSAF-PHITSVPRP--SSQGVPSMQT |
| Populus_trichocarpa | TISVGNVPTATVKVEPSTITSMANGPTF-PHNPSVPRP--ASQGVPILQT |
| Sorghum_bicolor | PV-VGGISTATVKLEPATMPPIVSAPAF-SHVTPISNV--ASQGISALQT |
| Triticum_aestivum | P-------TANVKVEPTTIPPMVSAPAF-SHMTPISNV--ASQGVSAMQT |
| Vitis_vinifera | TIAVGAVPTATVKVEPSTVTSITSGPGF-PHIPSVPRA--ASQGVPSLQT |
| Zea_mays | PI-VGGISTTSVKVEPTTMPPIVSAPAF-SHVTPISNV--ASQGISALQT |
| | * . : .* ..: . :. :*: |
| Arabidopsis_thaliana | SSASPVSQDMVSNAENAPDIKPVVVSGMTPPLRTGPPGGANVNLLNNLSQ |
| Brachypodium_distachyon | SSPSIISQETNVANEILQEHKPLVNP-IQQQVRPGGP--ANVSILNNLSQ |
| Carica_papaya | SSPLSTSQDMIINNDNVQEVKPIVSS-MTQPLRPMAPGAANVSILNNLSQ |
| Cucumis_sativus | SSPL-SSQEMITNNENTQDLKPLVTG-VPQPVRSLGP--ANVSILNNISQ |
| Eucalyptus_grandis | SSPSSVSQDMVTNNESVQDLKPVVSAGIPQTLRPVVP--ANSNILNNLSQ |
| Glycine_max | SSPSSVSQDIITNNETAQDTKPTVSM-L--PLRPVNPVQANVNILNNLSQ |
| Medicago_trunculata | SSPSSVSQDIMTSNENAMDTKPIVSM-L-QPIRPVNPAQANVNILNNLSQ |
| Mimulus_guttatus | SSPISTPQEVIPINENIPEVKPLVAS-SAPPIRPAGGAAANVRILNDMAQ |
| Oryza_sativa | PSPSLISQETNLANDSVQEHKPLINP-IQQSIRPGGP--ANVSILNNLSQ |
| Populus2 | SSPSTTTQDMATNGENAQDLKPNVSV-TTQSARSAPPAAANVNILNNLSQ |
| Populus_trichocarpa | SSPSTTTQDMATSGDDVQDLKPNVSV-MTQSARPGPPAAANVSILNNISQ |
| Sorghum_bicolor | SSPSLISQEANMANDNVQEHKPIINP-VQQPVRPGG----HGSLLNNLSQ |
| Triticum_aestivum | SSPSIISQETNVANEILQEHKPLVNP-IQQQVRPGGP--ANVSILNNLSQ |
| Vitis_vinifera | SSPSSTSQEMISNGDNVQDLKPIVSG-ISQTLRPVVPAAANVSILNNLSQ |
| Zea_mays | SSPSLISQEANMGNDNVQEHKPIINP-VQQPIRPGG----HGSLLNNLSQ |
| | .*. .*: : : ** : *. : :**:::* |
| Arabidopsis_thaliana | VRQVMSSAALA--GA-AS----SVGQSAVAMHMSNMISTGMATSLPPSQT |
| Brachypodium_distachyon | HRHS-----LS--GA-TSMG-PNMGATPIQVHMSNMISSGMTST------ |
| Carica_papaya | VRQVMNSAALT--GG-TSAGLQSMGQTPVAMHMSNMISSGMASSVPAAQN |
| Cucumis_sativus | AR-VMSTAALN--GG-TSIGLPSMGQTPIAMHVSNMISSGMGSSVPAAQN |
| Eucalyptus_grandis | VRQVVNSATLT--GG-TSIGLQSMGQNPMAMHMSNMISSGMQSTVPAAQT |
| Glycine_max | ARQVMNSAALS--GG-TSMGLPSMGQTPVAMHMSNMISSGMTSSVPAAQN |
| Medicago_trunculata | ARQVM---ALS--GG-TSMGLQSMGQTPVAMHMSNMISSGTTSSGPTGQN |
| Mimulus_guttatus | ARQALAAAAAGGGGG-ASIGIPSMGGGT--TMLSNMISSGMMTSS----- |
| Oryza_sativa | HRSV-----A------------------------TIISGGMPG------- |
| Populus2 | ARQVMNSAALS--GG-TSIGLPSINQTSVAMHMSSMISSGMTSSVPAAQT |
| Populus_trichocarpa | ARQVMNSAGLS--GG-TSLGLPSINQTSVAMHMSNMISSGMASSVPAAPT |
| Sorghum_bicolor | VRLM-NSTSLG--GGATSMGLPNMGATPIQVHMSNMISSGMTST------ |
| Triticum_aestivum | HRHS-----LT--AA-TSMG-PNMGATPIQVHMSNMISSGMTST------ |
| Vitis_vinifera | ARQVMHSAALS--GG-TSIGLQSMGGTSMAMHMSNMISSGMASSVPATQT |
| Zea_mays | VRLM-NSTSLG--GGATSMGLPNMGATPIQVHMSNMISSGMTST------ |
| | * .:** * |
| Arabidopsis_thaliana | VFSTGQQGITSMAGSGALMGSAQTGQSPGPNNAFSPQTTSNVASNLG-VS |
| Brachypodium_distachyon | -----PSVISSMSGTVQPTGAQQLVQNTALGSFGSNNSTVSGNSNVA-VS |
| Carica_papaya | VFSSGQSVITSITGSGTITGTSQVAQNSGLNTFTSATPSVSGNANLG-IS |
| Cucumis_sativus | VFSSGQSGMTSINGSSTL---SQVAPNSGISSLTSGNNNISGNPNIA-TS |
| Eucalyptus_grandis | GYSSGQSGITSVTGPGTLSGTIPSAQNPGLGSFASATSNITGNSNIG-MS |
| Glycine_max | VFSSGQSGITSMTSSGPLTVPAQVGQNSGLGSLTSNTSNLSSSSNIG-IS |
| Medicago_trunculata | VFSSGPSVITS---SGSLTASAQVGQNSGLSSLTSATSNSSP--NTG-IS |
| Mimulus_guttatus | SMSSGPSIVTSISGPLPMSANGQVAQNN------------SGNSGIAMSS |
| Oryza_sativa | --------I-PMSGTGQSIGSQQVVQNTAFGSNT----PITGNSNIA-VS |
| Populus2 | VFSSGQPGVSSITGSGNLTGTTQITPNSGLGSFTSATSNMPGNSNLGGIS |
| Populus_trichocarpa | VFSSGQPGVSSITGSGTLTGTTQIGPNSGLGSFTSATSNVSGNSNLGRIS |
| Sorghum_bicolor | -----PSVISSMSGPGQPIGTQQMVQSTALGSFGSNTSTVSGNSNVA-VS |
| Triticum_aestivum | -----PAVISSISGTVQPIGAQQLAQNTALGSFGSNTSTVSCNSNIA-VS |
| Vitis_vinifera | VFSSGQSAVSSITGSGTLAGTAQVAQNSALGSFTSATSNMSVNSNLG-IS |
| Zea_mays | -----PSVISSMSGPGQPISTQQMVQSTALGSFGSNTPTVTGNSTIA-VS |
| | : . . . . * |
| Arabidopsis_thaliana | QP-------------MQ-GM--NQGS--HS---GA-MMQGGISMNQNMMS |
| Brachypodium_distachyon | SSLANIQNNIATAQSVP-PM--AQGGL-MSGSHGSQSGQGGIGTNQNMIS |
| Carica_papaya | QSMATIQGGASVAQAVT-GM--NQGS--HS---GAQMVQSGIGMNQNTMT |
| Cucumis_sativus | QAVGNLQGSVSVSQSVP-GI--SQGN--LA---GTQVVQSGIGVSQNVMS |
| Eucalyptus_grandis | QPTGNLQVGPGMGQTAP-GM--SQGS--LS---GSQMVQNGVGMNPNMMS |
| Glycine_max | QPLGNLQGVVSIGQQVS-GM--SQGN--LS---GAQMVQGGVSMNQNVMS |
| Medicago_trunculata | QPIANLQGGVSMGQQVP-TM--NPGN--LS---GAQMVQGGVNMNQNVIN |
| Mimulus_guttatus | QPLSGLQGNVGPGQT---------GNI-PV---VPQMVQTGMGMNANMMS |
| Oryza_sativa | SSLGGIQSNIGI--SGP-PV--TQGGS-MG---STQLGQGGINTNQNMIS |
| Populus2 | QPMGNLHAGVSISQSAMSGLGTNPNTMSGS---GAQMVQSGMGANPNTMS |
| Populus_trichocarpa | QPMGNLQAGVSIGQSAP-GM--SQGN--IS---GAQMVQSGMGANPNTMS |
| Sorghum_bicolor | SSLTNNQSSMGMGQSVQ-PV--AQGGL-VA---GSQLGQGGIGANQNVMS |
| Triticum_aestivum | SSLANIQSNMAMGQSVP-SM--AQGGL-MA---GPQSGQGGIGTNQNMIN |
| Vitis_vinifera | QPL------------IP-GM--SQGN--LP---GGQMVQSGIGMNQNMMS |
| Zea_mays | SSLTNNQSSMGMGQSVQ-SV--AQGGL-VS---GSQLGQGGIVANQNVMS |
| | .. . * *: . * :. |
| Arabidopsis_thaliana | GLGQGNVSSGTGGMMPTPGVGQQAQSGIQQLGGSNSS--APNMQLSQPSS |
| Brachypodium_distachyon | SLGPTAISSA-PAMMPTPGMTQQ--AGVNSLSVNNSS--AMNMPMPQQPN |
| Carica_papaya | GVGPSSVSSGTGAMIPTSGMSQQVQPGMQPLGVNNSS--AVNMPLSQHAS |
| Cucumis_sativus | NLTQPGVSSGNGTMIPTPGMPQQVQNGMHSLGMNN-A--AANMPLPQHSA |
| Eucalyptus_grandis | SLGPTAPSSGSGTMMPTPGMPSQGQSGMSSINGNSNPGPGASMPLSQQSS |
| Glycine_max | GLGQSVVSSGTGTMIPTPGMSQPVQSVMQPL--VNNA--AANMPLSQQTS |
| Medicago_trunculata | GLGQSGVSSGTGAMIPTPGIPQQVQSGMQPL-VNNAA--AANMTLTQQTA |
| Mimulus_guttatus | GGGGAS------------GMSQQ---GQPPGGMNSNN--VVN-TLNQQTS |
| Oryza_sativa | SLGTTTVSSA-PAMMPTPGMAQQ--AGVNSLGVTNSS--AMNMPIVQHPN |
| Populus2 | GLGASGVSSGSNTMIPTPGMSQQVQSGMQPLGANNNS--AANLPLSQQTA |
| Populus_trichocarpa | GLGPSGVSSGLNTMIPTPGMSQQVQSGMQPLGANNNS--ADNLSMSQQTA |
| Sorghum_bicolor | SLGSTAISSA-PAMMPTPGMVPQ--TGVNSLGVNNNP--AMNMPIPQHAN |
| Triticum_aestivum | NLGTTAINST-PTMMPTPGMVQQ--TGVNALSANNSS--AMNMPLAQHPN |
| Vitis_vinifera | GLGPSGISSGTGTMIPTPGMSQQVQPGMPSLGVNNNA--AANMPLPQQTS |
| Zea_mays | TLGPTAISST-PAMMPTPGMVPQ--TGVNSLGVNNNS--AMNMPITQQHA |
| | *: . . : * |
| Arabidopsis_thaliana | GAM----------QTSQSKYVKVWEGNLSGQRQGQPVLITRLEGY----- |
| Brachypodium_distachyon | GQ-------------QQSKYVKIWEGTLSGQRQGQPVFICKLEGY----- |
| Carica_papaya | SSLA---------SSSQSKYVKVWEGNLSGQRQGQPVFITRLEGY----- |
| Cucumis_sativus | GALQ---------QQAQSKYVKFWEGNLSGQRQGQPVFITRMEAFVVIRK |
| Eucalyptus_grandis | SAL----------QSAQSKYVKVWEGNLSGQRQGQPVLITRLEGY----- |
| Glycine_max | GGM----------QSAQSKYVKVWEGSLSGQRQGQPVFITKLEGY----- |
| Medicago_trunculata | S--------------SQSKYIKVWEGSLSGQRQGQPVFITKLEGY----- |
| Mimulus_guttatus | ATM----------QSAQSKYVKVWEGNLSGQRQGQPVFITRLEGY----- |
| Oryza_sativa | AQQQQQQQQQQQQQQPPPKYVKIWEGTLSGQRQGQPVFICKLEGY----- |
| Populus2 | GGL----------QPAQSKYVKVWEGNLSGQRQGQPVFITRLEGY----- |
| Populus_trichocarpa | GGL----------QPPQSKYVKVWEGNLSGQRQGQPVFITRLEGY----- |
| Sorghum_bicolor | -AQ-----------QPPPKYVKIWEGTLSGQRQGQPVFICKLEGY----- |
| Triticum_aestivum | GQ-------------QPSKYVKIWEGTLSGQRQGQPVFICKLEGY----- |
| Vitis_vinifera | GAM----------QTAQSKYVKVWEGNLSGQRQGQPVFITRLEGY----- |
| Zea_mays | NAQ-----------QPPPKYVKIWEGTLSGQRQGQPVIICKLEGY----- |
| | .**:*.***.**********:* ::*.: |
| Arabidopsis_thaliana | -RSASASDSLAANWPPTMQIVRLISQDHMNNKQYVGKADFLVFRAMSQHG |
| Brachypodium_distachyon | -RSGTASDTLAADWPETMQIVRLIAQEHMNNKQYVGKADFLVFRTLNQHG |
| Carica_papaya | -RSSSASDTLAANWPPTMQIVRLISQDHMNNKQYVGKADFLVFRAMNQHG |
| Cucumis_sativus | LRFLLHDVKLAANWPQTMQIVRLISQDHMNNKQYVGKADFLVFRAMNQHG |
| Eucalyptus_grandis | -RSASASETLAANWPPTMQIVRLISQDHMNNKQYVGKADFLVFRAMNQHG |
| Glycine_max | -RNSSASETLAANWPPVMQIVRLISQDHMNNKQYVGKADFLVFRAMNPHG |
| Medicago_trunculata | -RSSSASETLAANWPPVMQIVRLISQDHMNNKQYVGKADFLVFRAMNPHG |
| Mimulus_guttatus | -RSASASETLAANWPSTMQIVRLISQDHMNNKQYVGKADFLVFRAMNQHG |
| Oryza_sativa | -RSGTASETLAADWPETMQIVRLIAQEHMNNKQYVGKADFLVFRTLNQHG |
| Populus2 | -RSASASETLAANWPPTMQIVRLISQDHMNNKQYVGKADFLVFRAMNQHG |
| Populus_trichocarpa | -RSASASETLAANWPPTMQIVRLISQDHMNNKQYVGKADFLVFRAMNQHG |
| Sorghum_bicolor | -RSGTASETLAADWPETMQIVRLIAQEHMNNKQYVGKADFLVFRTLNQHG |
| Triticum_aestivum | -RSGTASGTLASDWPETMQIVRLIAQEHMNNKQYVGKADFLVFRTLNQHG |
| Vitis_vinifera | -RSASASESLAANWPATMQIVRLISQDHMNNKQYVGKADFLVFRAMNQHG |
| Zea_mays | -RSGTASETLAADWPETMQIVRLIAQEHMNNKQYVGKADFLVFRTLNHHG |
| | * . .**::** .*******:*:*****************::. ** |
| Arabidopsis_thaliana | FLGQLQDKKL-----------------------------------CAVIQ |
| Brachypodium_distachyon | FLGQLQEKKL-----------------------------------CAVIQ |
| Carica_papaya | FLGQLQEKKL-----------------------------------CAVIQ |
| Cucumis_sativus | FLGQLQEKKL-----------------------------------CAVIQ |
| Eucalyptus_grandis | FLGQLQEKKL-----------------------------------CAVIQ |
| Glycine_max | FLGQLQEKKL-----------------------------------CAVIQ |
| Medicago_trunculata | FLGQLQEKKL-----------------------------------CAVIQ |
| Mimulus_guttatus | FLGQLQEKKL-----------------------------------CAVIQ |
| Oryza_sativa | FLGQLQEKKL-----------------------------------CAVIQ |
| Populus2 | FLGQLQEKKL-----------------------------------CAVIQ |
| Populus_trichocarpa | FLGQLQEKKL-----------------------------------CAVIQ |
| Sorghum_bicolor | FLGQLQEKKL-----------------------------------CAVIQ |
| Triticum_aestivum | FLGQLQEKKL-----------------------------------CAVIQ |
| Vitis_vinifera | FLGQLQEKKL-----------------------------------CAVIQ |
| Zea_mays | FLVQLQEKKLSNFVILLRFIYCRFIHLVFCQLLYTVLISNWSWFQCAVIQ |
| | ** ***:*** ***** |
| Arabidopsis_thaliana | LPSQILLLSVSDKACRLIGMLFPGDMVVFKPQIPNQQQQQQQQLHQQQQQ |
| Brachypodium_distachyon | LPSQILLLSVSDKAGRLIGMLFPGDMVVFKPQVSTQQPQMQQQQQLQQQH |
| Carica_papaya | LPSQTLLLSVSDKACRLIGMLFPGDMVVFKPQLPSQQQQQQQQQMQ---Q |
| Cucumis_sativus | LPSQTLLLSVSDKACRLIGMLFPGDMVVFKPQISSQQQQQHQMQQQQ--M |
| Eucalyptus_grandis | LPSQTLLLSVSDKACRLIGMLFPGDMVVFKPQIPS--------------- |
| Glycine_max | LPSQTLLLSVSDKAFRLIGMLFPGDMVVFKPQLSSQQQQQMQQQHQQMQN |
| Medicago_trunculata | LPSQTLLLSVSDKACRLIGMLFPGDMVVFKPQLSGQQQQQQMQQ-QQ--M |
| Mimulus_guttatus | LPSQTLLLSVSDKAHRLIGMLFPGDMVVFKPQIPNNNNNNNNNQQQQQQM |
| Oryza_sativa | LPSQTLLLSVSDKAGRLIGMLFPGDMVVFKPQVPTQQPPMQQQQLQQQQN |
| Populus2 | LPSQTLLLSVSDKACRLIGMLFPGDMVVFKPQISSQQQQMQQQHHQQMQQ |
| Populus_trichocarpa | LPSQTLLLSVSDKACRLIGMLFPGDMVVFKPQISSQQQQMQMQRQQ---Q |
| Sorghum_bicolor | LPSQTLLLSMSDKAGRLIGMLFPGDMVVFKPQVPTQQTPMQQQQLQQQQL |
| Triticum_aestivum | LPSQTLLLSVSDKAGRLIGMLFPGDMVVFKPQVSTQQPQMQPQQQ-QQQQ |
| Vitis_vinifera | LPSQTLLLSVSDKACRLIGMLFPGDMVVFKPQITNSQQQQHQQL----QQ |
| Zea_mays | LPSQTLLLSMADKAGRLIGMLFPGDMVVFKPQASTQQTPMQQQQLQQFQQ |
| | *********::*** ***************** . |
| Arabidopsis_thaliana | QQ--QIQQQ---------------Q--QQQ---QHLQ-QQQMPQLQQQQQ |
| Brachypodium_distachyon | HPL---HQQ---------------QLQ----QL---Q---------QQ-- |
| Carica_papaya | QQ--QMQQQ------------------Q-H---PQLQQ-QQLPQLQQQQQ |
| Cucumis_sativus | QQ--QLQQQ------------------QQH-QLSQLQQQHQLPQMQQQQQ |
| Eucalyptus_grandis | -------------------------------------------------- |
| Glycine_max | QQQH-LPQL---------------Q--QQQ-QLPHMQ-QQQLPQLQQQQQ |
| Medicago_trunculata | QQHQ---QM---------------Q--SQ---------QQHLPQLQQQMQ |
| Mimulus_guttatus | QV--QLQQQ--------------------Q-MQVQ---------LQQQQI |
| Oryza_sativa | QLQ---QQN---------------QLHQQH-QL---Q-----P---QN-- |
| Populus2 | QQHPQLQQL---------------Q--HQQ-QLPQLQQQQQLSQLQQQQQ |
| Populus_trichocarpa | QQ--QMQQQ---------------Q--QQQ-QHP---------Q------ |
| Sorghum_bicolor | QQQ-QLQQQ---------------QLQQQH---MHMQ-QQGLP-LQQSQM |
| Triticum_aestivum | Q------------------------------------------------- |
| Vitis_vinifera | QQIPLLQQQ--------------------Q-QLAPLQQQQQLPQLQQQQ- |
| Zea_mays | QQQ-QLQQHMHMQPQGLPLQQSQMQLQQQQPQMQPMP-QQQPSQMQQQMQ |
| Arabidopsis_thaliana | -----QHQQQQQQQ-HQLSQLQHHQ--------------------Q--QQ |
| Brachypodium_distachyon | QLQQQH-MQM---Q-PQGQQLQQQQQI----------------------Q |
| Carica_papaya | -----H-PQQ---Q--------Q--------------------QQP--QQ |
| Cucumis_sativus | -----Q-QQQ---Q-PQLSHMQQQQ----Q-------------QQQ--QQ |
| Eucalyptus_grandis | -------------------------------------------------- |
| Glycine_max | -----QLPQLQQQQ-QQLPQLQQQQ----QQL------PQ---LQQ--QQ |
| Medicago_trunculata | -----Q----QQQQ-QQLPQLQQNQ----Q-------------LSQ--IQ |
| Mimulus_guttatus | ----------QQQQ-QPIMQIQQQQ--------------------Q--QQ |
| Oryza_sativa | QLQQQHQLQQ---Q-LQQQQLQQHMQLQTQGLPLQQQQSQGHPLQQ-QQM |
| Populus2 | -----QLSQLQQQQPQQLSQLQQQQ---PQQL------PQ---LQ---QQ |
| Populus_trichocarpa | ---------LQQQQ----------------------------------H- |
| Sorghum_bicolor | QLQQQQQPQMQPMQ-QQPTQMQHQQQQPSQMQPMQHQQPQPQQMQSMQHQ |
| Triticum_aestivum | -LQQQH-HQ----------------------------------------- |
| Vitis_vinifera | -----QLPQLQQQQ--Q------LQ---QQQH------PQ---LQ---Q- |
| Zea_mays | SMQQQMQPMQQQMQ-HQQQQMQHQQQQM----------QQMQQQQQ-QQQ |
| Arabidopsis_thaliana | QQ-QQQQQQ-----QHQLTQLQHHHQQQQ-----QAS---PLNQ------ |
| Brachypodium_distachyon | QQMQQQQQQ-----QQQMQQMQHQQQQQQQ----------------IQ-- |
| Carica_papaya | QPLPQLQQQ-------QLPQLQQQQQQ----PL---------SQLQQQQ- |
| Cucumis_sativus | QPLPQIQQQQ----QSQLPQIQQQQQQQQQPQL---------SQLQNQQQ |
| Eucalyptus_grandis | -------------------------------------------------- |
| Glycine_max | QQLPQLQQQ-----QQQLPQLQQQQQQVQQQQL---------AQLQQQ-- |
| Medicago_trunculata | QQIPQLQQQ-----QQQLPQLQQQ-------------------------- |
| Mimulus_guttatus | QQIIQQQQQ-----QQQ--------------------------------- |
| Oryza_sativa | QQMQQQ-QQ-----QQQIQQMQQQQ------------------------- |
| Populus2 | QQLSQLQQQQLPQMQQQLPQMQQQQQLQQQQQMQQQQQMQQQQQLQQQ-- |
| Populus_trichocarpa | --------------QQQLPQL------------------------QQQ-- |
| Sorghum_bicolor | QQMQHQQQQ-----QQQMQPMQQ-----------------------QQ-- |
| Triticum_aestivum | --IQ--QQQ-----QQQMQQMQQQ-------------------------- |
| Vitis_vinifera | --------------QQQLPQLQQQ-----------------------Q-- |
| Zea_mays | QQQQQIQPQ-----QQQMQQMQQQQQQQMQP--------------QQQ-- |
| Arabidopsis_thaliana | -----MQQQT-------SPLNQMQQQTSP------L--NQMQ----Q-QQ |
| Brachypodium_distachyon | -QQQQMQQQQ-------QQMQQMQQQ-------------QPQ-----QLQ |
| Carica_papaya | -PLPQLQQQQ-------QQLPQLQQQ------------QQL--------P |
| Cucumis_sativus | PQLSQLQSQQ-Q-----PQLSQLQSQ------------QQIP----QMQQ |
| Eucalyptus_grandis | -------------------------------------------------- |
| Glycine_max | -QLPQIQQQQQL--PQLQQLPQLQQQQQ-------L--PQLQ----QLQP |
| Medicago_trunculata | -QLSQLQQQQ-------QQLPQLQQLQH-------------Q----Q-LP |
| Mimulus_guttatus | --MPQMQQQQ--------NQQQMQQQQN---------------------- |
| Oryza_sativa | -QMQQMQQQQ-------QQPQQLQQQQQPQMVGTGMGQQQPQMVGTGMQG |
| Populus2 | -QQQQLQ-Q--------QQQQQMQQQQQ----------QQLQ----Q-MQ |
| Populus_trichocarpa | -QLSLLQ-Q--------QQLSLLQ-QQ-----------QQLP----Q-L- |
| Sorghum_bicolor | -QMQHMQHQQ-------QQMQQMQQQQQQ------M--QQMQ----PQQQ |
| Triticum_aestivum | ------------------------QQ-------------QPQ-----QLQ |
| Vitis_vinifera | -QLPQLQQQ--------HQLSQLQPQQQ-------H--QPLQ----Q-QQ |
| Zea_mays | -QMQQMQQQQQQMQPQQQQMQQMQQQQ-----------QQMQ----PQQQ |
| Arabidopsis_thaliana | QPQQMVMG--GQ-AFA-QAPG-RSQ-QG------GGGGQ------PNMPG |
| Brachypodium_distachyon | QQPQMVGPGMGQQQFM-QGHG-RAV-QMMQ-GKIAPQGP------GSMSG |
| Carica_papaya | RQQQIVGSSMAP-VYP----T-RAQ-LLA---QTSSQGP------PDIPG |
| Cucumis_sativus | QQQQMVAGGINQ-SYV-QG-G-RSQ-LVSQ-GQCSSQGQ------SNMSA |
| Eucalyptus_grandis | ------GSGMGQ-GYV-QGPG-RSQ-LMPQ-GPVSSQGL------PNMPG |
| Glycine_max | QQQQMVGSGMGQ-AYV-QGPG-RSQ-LVSQ-GQVSSQGA------TNIGG |
| Medicago_trunculata | QQQQMVGAGMGQ-TYV-QGPGGRSQ-MVSQ-GQVSSQGA------TNIGG |
| Mimulus_guttatus | -QQQ-----MNQ-AYMSQGGGQRSQ-IMSQGGQASSQGPP-----PTMPG |
| Oryza_sativa | QQPQMVGAGMGQ-QYM-QGHG-RTVQQMMQ-GKMAPQGPGSMPGAGSMPG |
| Populus2 | QQQQMVGTGMGQ-TYV-QGPG-RSQ-LVSQ-GQVSSQGP------ANMSG |
| Populus_trichocarpa | QQQQMAGTGMGQ-TYV-QGPG-RPQ-LVSQ-GQVSSQGP------ANMPG |
| Sorghumv bicolor | QQPQMVGTGMGQ-QFM-QGQN-RAV-QMMQ-GKIMPQGP------GSMPG |
| Triticum_aestivum | QQPQMVGTGMGQQQFM-QGHG-RAV-QMMQ-GKIAPQGP------GNMSG |
| Vitis_vinifera | QPQQNVGTGMGQ-AFV-QGPG-R-----------SSQGP------PNMPG |
| Zea_mays | QQPQMVGTGMGQ-QFM-QGHN-RAV-QMMQ-GKITPQGP------GSMPG |
| | : * * : . |
| Arabidopsis_thaliana | AGFM-G |
| Brachypodium_distachyon | GGYL-S |
| Carica_papaya | AGFM-G |
| Cucumis_sativus | GNFM-G |
| Eucalyptus_grandis | GGFM-S |
| Glycine_max | GGFM-S |
| Medicago_trunculata | GNFM-S |
| Mimulus_guttatus | GPFI-N |
| Oryza_sativa | GGYL-S |
| Populus2 | GGFMSQ |
| Populus_trichocarpa | GGFM-S |
| Sorghum_bicolor | GGFL-P |
| Triticum_aestivum | GGYL-S |
| Vitis_vinifera | GAFL-G |
| Zea_mays | GGFL-P |
| | . :: |

Underlined amino acid sequences correspond to the 4 conserved peptides within the vWF-A domain and the 3 conserved peptides within the ACID domain.
* = amino acid identity;
: = conservative amino acid substitutions;
. = partially conservative amino acid substitutions.

TABLE 2
Alignment of amino acid sequences of Med 18 polypeptides

| | |
|---|---|
| Arabidopsis_lyrata | MS-----------------------MECVVQGIIETQHVEALEILLQGLC |
| Arabidopsis_thaliana | MS-----------------------MECVVQGIIETQHVEALEILLQGLC |
| Brachypodium_distachyon | -------------------------MECVVQGIIETQHVEALEVLLQGLS |
| Carica_papaya | -------------------------MECVVQGIIETQHVEALEILLQGLC |
| Cucumis_sativus | -------------------------MECVVQGIIETQHVEALEILLQGIC |
| Eucalyptus_grandis | -------------------------MECVVQGIIETQHVEALEILLQGLC |
| Glycine_max_1 | -------------------------MECVVQGIIETQHVEALEILLQGLC |
| Glycine_max_2 | MDLSKVLDCINNICISCVMQLSIVDMECVVQGIIETQHVEALEILLQGLC |
| Glycine_max_3 | -------------------------MECVVQGIIETQHVEALEILLQGLC |
| Manihot_esculenta | -------------------------MECVVQGIIETQHVEALEILLQGLC |
| Mimulus_guttatus | -------------------------MECVVQGIIETKHVEALEILLQGLC |
| Oryza_sativa | -------------------------MECVVQGIIETQHVDALEVLLQGLS |
| Populus_trichocarpa | -------------------------MECVVQGIIETQHVEALEILLQGLC |
| Ricinus_communis | -------------------------MECVVQGIIETQHVEALEILLQGLC |
| Sorghum_bicolor | -------------------------MECVVQGIIETQHVEALEVLLQGLS |
| Vitis_vinifera | -------------------------MECVVQGIIETQHVEALEILLQGLC |
| Zea_mays | -------------------------MECVVQGIIENQHVDALEVLLQGLS |
| | **********.:**:***:****:. |
| Arabidopsis_lyrata | GVKRERLRVHELCLKSGPNLGVVSSEVRLLCDLDQPEPTWTVKHVGGPMR |
| Arabidopsis_thaliana | GVQRERLRVHELCLRSGPNLGVVSSEVRLLCDLDQPEPTWTVKHVGGAMR |
| Brachypodium_distachyon | GVPKERVRVHELCLKSGPNLGVVPSEVRLLCDLAQPTPSWTIRHVGGAMR |
| Carica_papaya | GVHRERLRVHEICLKSGPNLGIVSSEVRLLCDLEQPEPTWIVKHVGGAMR |
| Cucumis_sativus | GVPRERLRLHEICLKSGPSLGAVASEVRILCDLEQAEPTWIVKHIGGAIR |
| Eucalyptus_grandis | GVQKERLRIHELCLKSSPNLGAVPSEVQILCDLAQPEPSWIVRHVGGSMR |
| Glycine_max_1 | GVQRERLRIHEICLKSGQHLGTVASEVRLLCDLEQAEPSWIVKHVGGAMR |
| Glycine_max_2 | GVQRERLRIHEICLKSGPHLGTVASEVRLLCDLEQAEPSWIVKHVGGAMR |
| Glycine_max_3 | GVQRERLRIHEICLKSGPHLGTVASEVRLLCDLEQAEPSWIVIHAGGAMR |
| Manihot_esculenta | GVHREDFRIHELCLKSGPNLGLVASEVRLLFDLYQAEPTWTVKHVGGAMR |
| Mimulus_guttatus | GVHRESLRVHELCLKSVPNLGLVSSEIRLLCDLEQPEPTWIVRHVGGALR |
| Oryza_sativa | GVPKERVRVHELCLKSGPNLGVVPSEVRLLCDLAQSTPSWTIRHVGGAMR |
| Populus_trichocarpa | GVHKEHLRVHELYLKSGPNLGHVTSEVRLLCNLEHPEP-WTVKHVGGALR |
| Ricinus_communis | GVHRERLRIHELCLKSGPNLGIVASEVRLLCDLEQPEPTWTVKHVGGAMR |
| Sorghum_bicolor | GVPKERVRVHELCLKSGPNLGVVPSEVRLLCDLAQPTPSWTIRHVGGAMR |
| Vitis_vinifera | GVHRERLRIHELCLKSGPNLGLVASEVRVLCDLEQPEPTWIVRHIGGAMR |
| Zea_mays | GVPKERFRVHELCLKSGPNLGVVPSEVRLLCDLAQPIPSWTIRHVGGAMR |
| | ** :* .*:**: *:* ** *.**:::* :* :. * * : * **.:* |
| Arabidopsis_lyrata | GAGADQISVMVRTMIESKVSKNALRMFYALGYKLDHELLKVGFAFHFQRT |
| Arabidopsis_thaliana | GAGADQISVLVRNMIESKVSKNALRMFYALGYKLDHELLKVGFAFHFQRT |
| Brachypodium_distachyon | GAGAEQISVLVRTIVESKASNNVLRYFYGIGYKLDHEVLKVGFAFRFQRG |
| Carica_papaya | GAGAEQISVLVRTMVESKVSKNVLRLFYALGYKLDHELLRVGFTFHFQRG |
| Cucumis_sativus | GAGAEQLSVLVRTMVESKVSKNVLRLFYALGYKLDHELLRVGFAFRFHRG |
| Eucalyptus_grandis | GTGAEQISVLVRPMVESKASKNVLRFFYALGYKLDHELLRVGFAFHFQRG |
| Glycine_max_1 | GAGAEQISVLVRSMVESKTSKNALRMFYILGYKLDHELLRVGFSFNEHRG |
| Glycine_max_2 | GAGAEQISVLVRSMVESKTSKNALRMFYILGYKLDHELLRVGFSFNEHRG |
| Glycine_max_3 | GAGAEQISVLVRSMVESKTSKNALRMFYILGYKLDHELLRVGFSFNEHRG |
| Manihot_esculenta | GAGADQISVLVRTMVESKASKNVLRLFYALGYKLDHELLRVGFAFHFQRG |
| Mimulus_guttatus | GAGADQISVLVRNMMESKVSKNALRLFYALGYKLDHELLRVGFTFQFHRG |
| Oryza_sativa | GAGAEQISVLVRSIVESKASNNVLRYFYGIGYKLDHEVLKGGFAFRFHRG |
| Populus_trichocarpa | GAGAEQISVLVRNMVESKASKNVLRLFYALGYKLDHELLRVGSAFHFKRG |
| Ricinus_communis | GAGAEQISVLVRTMVESKASKNVLRLFYALGYKLDHELLRVGFAFHFQRG |
| Sorghum_bicolor | GAGAEQISVLVRTIVESKASKNVLHYFYILGYKLDHELLKIGFAFRFHRG |
| Vitis_vinifera | GSGAEQISVLVRTMVESKVSKNVLRLFYALGYRLDHELLRVGFAFHFQRG |
| Zea_mays | GAGAEQISVLVRTVVESKASKNVLHYFYILGYKLDHELLKSGFAFREHRG |
| | *:**:*:**:** ::***.*:*.*: ** :**:****:*: * :*.*:* |
| Arabidopsis_lyrata | AHISVSVSSVNKMPKVHAI--DEAVPVTPGMQIVDVTAPATPENYSEVAA |
| Arabidopsis_thaliana | AHISVSVSSVNKMPKVHAI--DEAVPVTPGMQIVDVTAPATSENYSEVAA |
| Brachypodium_distachyon | AQFTVTVTSANKMPKLHAT--DEAVAVTPGIQLVEITAPAAANNYNDVAS |
| Carica_papaya | AQIKVSVSSINKMLKLHAT--DEAVPVTPGIQLVEVTAPATSENYAEVVA |
| Cucumis_sativus | AHITVTVSSVNKMLKLHST--DEAVPVTPGIQLVEVTAPATSDNYSEVAG |
| Eucalyptus_grandis | VQITVTVSSVNKMLKLHAT--DEAVPVTPGMQLVEVTAPATSENYTEVVA |
| Glycine_max_1 | AQITVTVSSINKMLKLHAT--DEAVPVTPGIQMVEVTAPATAETYTEVAA |
| Glycine_max_2 | AQITVTVSSINKMLKLHAT--DEAVPVTPGIQMVEVTAPATAETYAEVAS |
| Glycine_max_3 | AQITVTVSSINKMLKLHAT--DEAMPVTPGIQMVEVTAPATAETYAEVAS |
| Manihot_esculenta | ARITVAVSSVNKMLKLHATDGDEAVPVTPGIQLVEVTAPATSENYSEVVA |
| Mimulus_guttatus | AQITVTVSSVNKMLKLHST--DEAVPVTPGIQLVEVTAPASSENYTEVVA |
| Oryza_sativa | AQITVTVTSVSKMTKLHAT--NEAVPITPAIQLVEITAPAAADNYNDVVS |
| Populus_trichocarpa | AWITVTVSSINKMLKLHAI--DDTMPVTLGIQVVEVTAPATSENYSEVAA |
| Ricinus_communis | ARITVRVSSVNKMLKLHAT--DEAVPVTPGIQLVEVTAPATPENYSEVVA |
| Sorghum_bicolor | AQITVTVTSTNKMPRLHAT--DEAVPVTPGIQLVEITAPAAADNYNDVVS |
| Vitis_vinifera | ASITVTVSSVNKMLKLHAT--DEAVPVTPGIQLVEVTAPASSENYSEVAA |
| Zea_mays | AQITVTVTSANKMSRLHAT--DEATPVTPAIQLVEITAPAAADNYNDVVS |
| | . :.* *:* .** ::*: ::: .:* .:*:*::****:.:.* :*.. |
| Arabidopsis_lyrata | AVSSFCEFLAPLVHLSKPFISTGVVPTAAAAAASLMSDGG---GTTL |
| Arabidopsis_thaliana | AVSSFCEFLAPLVHLSKPSISTGVVPTAAAAAASLMSDGG---GTTL |
| Brachypodium_distachyon | SVTAFCEYLAPLLHLSKPGNSTGIVLTAGAAAASLMSSGG---AKTL |
| Carica_papaya | AVSSFCEYLAPLLHLSKPGISTGVVPTAAAAAASLMSDGG---GTTL |
| Cucumis_sativus | AVSTFCEYLAPLLHLSKPGISTGVVPTAAAAAASLLSDGA---GTTM |
| Eucalyptus_grandis | AVTSFCEHLAPLLHLSKPGISTGVVPTAAAAAASLMSDGG---GTTL |
| Glycine_max_1 | AVSSFCEYLAPLLHLSKPGISTGVVPTAAAAAASLMSDGG---STTL |
| Glycine_max_2 | AVSSFCEYLAPLLHLSKPGISTGVVPTAAAAAASLMSDGG---GTNL |
| Glycine_max_3 | AVSSFCEYLAPLLHLSKPGISTGVVP---AAAASLMSDGG---GTTL |
| Manihot_esculenta | AVSSFCEYLAPLLHLSKPGVSTGVVPTAAAAAASLMSDGG---GTTL |
| Mimulus_guttatus | AMSSFCEYLAPLLHLSKPGVSTGVVPTAAAAAASLMSDGG---GTAL |
| Oryza_sativa | AVTSFCEYLAPLLHLSKPGNSTGIVPTAGAAAASLMSSGGGGGGKTL |
| Populus_trichocarpa | AVSSFCEYLAPLLHLSKPGVSTGVVPTAAAAAASLMSDGG---GTTL |
| Ricinus_communis | AVSSFCEYLAPLLHLSKPGVSTGVVPTAAAAAASLMSDGG---GTTL |
| Sorghum_bicolor | AVSAFCEYLAPLLHLSKPGHSTGIVATAGAAAASLMSSGG---GKTL |
| Vitis_vinifera | AVSSFCEYLAPLLHLSKPGTSTGVVPTAAAAAASLMSDAG----VTT |
| Zea_mays | AVTAFCEYLAPLLHLSKPGHSTGIVPTAAAAAASLMSSGG---GKTL |
| | ::::***.****:***** ***:* ******:*... |

* = amino acid identity;
: = conservative amino acid substitutions;
. = partially conservative amino acid substitutions.

### CITED REFERENCES

1. Conaway R C, Conaway J W (1993) General initiation factors for RNA polymerase II. Annu Rev Biochem 62:161-190.
2. Kornberg RD (2005) Mediator and the mechanism of transcriptional activation. Trends Biochem Sci 30:235-239. mammalian Mediator complex. Trends Biochem Sci 30:256-263.
3. Backström S, Elfving N, Nilsson R, Wingsle G, Björklund S (2007) Purification of a plant mediator from Arabidopsis thaliana identifies PFT1 as the Med25 subunit. Mol Cell 26:717-729.
4. Cerdán PD, Chory J (2003) Regulation of flowering time by light quality. Nature 423:881-885.
5. Kidd B N et al. (2009) The mediator complex subunit PFT1 is a key regulator of jasmonate-dependent defense in Arabidopsis. Plant Cell 21:2237-2252.
6. Liu Q et al. (1998) Two transcription factors, DREB1 and DREB2, with an EREBP/AP2 DNA binding domain separate two cellular signal transduction pathways in drought- and low-temperature-responsive gene expression, respectively, in Arabidopsis. Plant Cell 10:1391-1406.
7. Sakuma Y et al. (2006) Dual function of an Arabidopsis transcription factor DREB2A in water-stress-responsive and heat-stress-responsive gene expression. Plant Cell 18:1292-1309.
8. Tran L-S et al. (2007) Co-expression of the stress-inducible zinc finger homeodomain ZFHD1 and NAC transcription factors enhances expression of the ERD1 gene in Arabidopsis. Plant J 49:46-63.
9. Rizhsky L et al. (2004) When defense pathways collide. The response of Arabidopsis to a combination of drought and heat stress. Plant Physiol 134:1683-1696.
10. Schaefer D G, Zrÿd J P (1997) Efficient gene targeting in the moss Physcomitrella patens. Plant J 11:1195-1206.
11. Kieber J J, Rothenberg M, Roman G, Feldmann K A Ecker JR (1993) CTR1, a negative regulator of the ethylene response pathway in Arabidopsis, encodes a member of the raf family of protein kinases. Cell 72:427-441.
12. Alonso J M, Stepanova A N (2003) T-DNA mutagenesis in Arabidopsis. Methods Mol Biol 236:177-188.

1. A method for producing a genetically modified plant with increased tolerance to water deficit and/or salt stress as compared to a corresponding non-genetically modified wild type plant, which comprises the following steps:
a. reducing or deleting the amount or activity of a Mediator subunit in a plant cell, a plant or a part thereof,
b. generating and/or selecting a genetically modified plant with increased tolerance to water deficit and/or salt as compared to a corresponding non-genetically modified wild type plant and growing under conditions which permit the development of the plant, wherein said plant is an annual or perennial crop plant.

2. The method of claim 1, the method steps further comprising:
c. selfing or crossing the genetically modified plant with itself or another plant, respectively, to produce seed; and
d. growing a progeny plant from the seed, wherein the progeny plant has increased tolerance and/or resistance to water deficit or salt stress.

3. The method of claim 1, wherein said subunit is a Med25 polypeptide comprising:
i. an activator-interacting domain comprising three peptides localised in sequential sequence of (a), (b) and (c) in the C-terminal half of said polypeptide, and wherein said peptides are:
[SEQ ID NO: 5];
(a) KY(V/I)KXWEGXLSGQRQGQPV(F/L/I)IX(K/R)(L/M)E
(G/A)(Y/F)
[SEQ ID NO: 6], and
(b): LA (A/S )XWPXXM QIVRLI(S/A)Q (D/E)HMNNKQYV
GKADFLVFR(T/A)(M/L)(N/S)XHGFLXQLQ(E/D)KKL
[SEQ ID NO: 7].
(c): CAVIQLPSQTLLLS (V/M)(S/A)DKAXRLIGMLFPGDMWF
KPQ
wherein X is any amino acid, and wherein the amino acid sequences of peptides (a), (b) and (c) are at least 80% identical to the corresponding peptide of a Med25 polypeptide having SEQ ID NO: 9.

4. The method of claim 3, wherein said Med25 polypeptide has an amino acid sequence having at least 80% amino acid sequence identity to a sequence selected from among SEQ ID NO's: 9, 11, 13, 15, 17, 19, 21, 23, 25, 27, 29, 31, 33, 35, and 37.

5. The method of claim 3, wherein said Med25 polypeptide has an amino acid sequence selected from among SEQ ID NO's: 9, 11, 13, 15, 17, 19, 21, 23, 25, 27, 29, 31, 33, 35, and 37.

6. The method of claim 1, wherein said subunit is a Med18 polypeptide and wherein the amino acid sequence of the polypeptide has at least 80% amino acid sequence identity to a sequence selected from among SEQ ID NO: 39, 41, 43, 45, 47, 49, 51, 53, 55, 57, 59, 61, 63, 65, 67, 69 and 71.

7. The method of claim 6, wherein said Med18 polypeptide has an amino acid sequence selected from among SEQ ID NO's 39, 41, 43, 45, 47, 49, 51, 53, 55, 57, 59, 61, 63, 65, 67, 69 and 71.

8. The method according to claim 2, comprising reducing or deleting the expression of at least one nucleic acid molecule, wherein said molecule is selected from:
a. a nucleic acid molecule encoding a Med 25 polypeptide or Med18 polypeptide according to claim 2,
b. a nucleic acid molecule having a nucleic acid sequence selected from among SEQ ID NO's: 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68 and 70.

9. The method according to claim 8, whereby the method comprises at least one step selected from among: (a) introducing into at least one plant cell a nucleic acid molecule encoding a ribonucleic acid sequence, which is able to form a double-stranded ribonucleic acid molecule, whereby a fragment of at least 17 nucleotides of said double-stranded ribonucleic acid molecule has a nucleic acid sequence having at least 50% nucleic acid sequence identity to a nucleic acid molecule selected from the group (i) or (ii) of claim 8; (b) introducing into at least one plant cell an RNAi or antisense nucleic acid molecule, whereby the RNAi or antisense nucleic acid molecule comprises a fragment of at least 17 nucleotides with a nucleic acid sequence having at least 50% nucleic acid sequence identity to a nucleic acid molecule selected from the group (i) or (ii) of claim 8; (c) introducing into at least one plant cell a nucleic acid construct able to recombine with and silence, inactivate, or reduce the activity of an endogenous gene comprising a nucleic acid molecule selected from the group (i) or (ii) of claim 8; and (d) introducing or detecting a non-silent mutation in an endogenous gene comprising a nucleic acid molecule selected from the group (i) or (ii) of claim 8.

10. The method according to claim 8, wherein reducing or deleting of the amount or activity of an Med25 polypeptide or Med18 polypeptide is caused by any one of:
a. a natural or induced mutation in an endogenous gene of the plant cell, the plant or a part thereof, and optionally in combination with ECO-TILLING or TILLING;
b. T-DNA inactivation of an endogenous gene;
c. site-directed mutagenesis or directed breeding of an endogenous gene,
wherein said endogenous gene comprises a nucleic acid molecule selected from the group (i) or (ii) of claim 8.

11. A method according to claim 8, said method comprising: (a) providing a vector comprising: (i) said nucleic acid molecule for introducing into at least one plant cell; (ii) a flanking nucleic acid molecule comprising one or more regulatory elements fused to said nucleic acid molecule, wherein the regulatory elements control expression of said nucleic acid molecule; and (b) transforming at least one cell of said plant with the vector to generate a transformed plant with increased tolerance to water deficit and/or salt stress as compared to a corresponding non-transformed wild type plant.

12. The method as claimed in claim 1, wherein the plant is any one of (a) a monocotyledous crop plant selected from the group consisting of Avena spp; Oryza spp.; Hordeum spp., Triticum spp.; Secale spp.; Brachypodium spp.,; Zea spp.; (b) a dicotyledenous crop plant selected from among Cucumis spp.,; Phaseolus spp., Glycine spp.,; Medicago spp.,; Brassica spp; and Beta spp., (c) a hardwood selected from among acacia, eucalyptus, hornbeam, beech, mahogany, walnut, oak, ash, willow, hickory, birch, chestnut, poplar, alder, maple, sycamore, ginkgo, a palm tree and sweet gum; (d) a conifer selected from among cypress, Douglas fir, fir, sequoia, hemlock, cedar, juniper, larch, pine, redwood, spruce and yew, (e) a fruit bearing woody plant selected from among apple, plum, pear, banana, orange, kiwi, lemon, cherry, grapevine, papaya, peanut, and fig and (f) a woody plant selected from among cotton, bamboo and a rubber plant.

13. A genetically modified annual or perennial crop plant having increased tolerance to water deficit and/or salt stress as compared to a corresponding non-genetically modified wild type plant, wherein said plant has a reduced amount or activity of a Mediator subunit, and wherein the genome of said plant comprises a genetic modification selected from any one of:
i) a non-silent mutation in an endogenous gene comprising a nucleic acid molecule encoding a Med18 polypeptide;
ii) a transgene inserted into said genome, said transgene comprising a nucleic acid molecule encoding a ribonucleic acid sequence, which is able to form a double-stranded ribonucleic acid molecule, whereby a fragment of at least 17 nucleotides of said double-stranded ribonucleic acid molecule has a homology of at least 50% to a nucleic acid molecule encoding a Med18 polypeptide;
iii) a mutation in an endogenous gene comprising a nucleic acid molecule encoding a Med18 polypeptide, induced by introducing into at least one plant cell a nucleic acid construct able to recombine with and silence, inactivate, or reduce the activity of the endogenous gene,
wherein said Med18 polypeptide has an amino acid sequence having at least 80% amino acid sequence identity to a sequence selected from among SEQ ID NO's 39, 41, 43, 45, 47, 49, 51, 53, 55, 57, 59, 61, 63, 65, 67, 69 and 71.

14. A genetically modified plant according to claim 13, wherein the plant is any one of (a) a monocot selected from the group consisting of Avena spp; Oryza spp.; Hordeum spp., Triticum spp.; Secale spp.; Brachypodium spp.; Zea spp.; (b) a dicot plant selected from among Cucumis spp.,; Phaseolus spp., Glycine spp.,; Medicago spp.,; Brassica spp; and Beta spp., (c) a hardwood selected from among acacia, eucalyptus, hornbeam, beech, mahogany, walnut, oak, ash, willow, hickory, birch, chestnut, poplar, alder, maple, sycamore, ginkgo, a palm tree and sweet gum; (d) a conifer selected from among cypress, Douglas fir, fir, sequoia, hemlock, cedar, juniper, larch, pine, redwood, spruce and yew, (e) a fruit bearing woody plant selected from among apple, plum, pear, banana, orange, kiwi, lemon, cherry, grapevine, papaya, peanut, and fig and (f) a woody plant selected from among cotton, bamboo and a rubber plant.

15. A genetically modified plant having increased tolerance to water deficit and/or salt stress as compared to a corresponding non-genetically modified wild type plant, wherein said plant is a hardwood selected from among acacia, eucalyptus, hornbeam, beech, mahogany, walnut, oak, ash, willow, hickory, birch, chestnut, poplar, alder, maple, sycamore, ginkgo, a palm tree and sweet gum having a reduced amount or activity of a Med25 polypeptide, and wherein the genome of said plant comprises a genetic modification selected from any one of:
i) a non-silent mutation in an endogenous gene comprising a nucleic acid molecule encoding a Med25 polypeptide;
ii) a transgene inserted into said genome, said transgene comprising a nucleic acid molecule encoding a ribonucleic acid sequence, which is able to form a double-stranded ribonucleic acid molecule, whereby a fragment of at least 17 nucleotides of said double-stranded ribonucleic acid molecule has a homology of at least 50% to a nucleic acid molecule encoding a Med25 polypeptide;
iii) a mutation in an endogenous gene comprising a nucleic acid molecule encoding a Med25 polypeptide, induced by introducing into at least one plant cell a nucleic acid construct able to recombine with and silence, inactivate, or reduce the activity of the endogenous gene,
wherein said Med25 has an amino acid sequence having at least 80% amino acid sequence identity to a sequence selected from among SEQ ID NO's: 9, 11, 13, 15, 17, 19, 21, 23, 25, 27, 29, 31, 33, 35, and 37.

16. A genetically modified plant of claim 15 having increased tolerance to water deficit and/or salt stress as compared to a corresponding non-genetically modified wild type plant, wherein said plant is a hardwood selected from among eucalyptus and poplar, wherein said Med25 has an amino acid sequence having at least 80% amino acid sequence identity to a sequence selected from among SEQ ID NO's: 19, 29 and 31.

17. A genetically modified plant of claim 14 having increased tolerance to water deficit and/or salt stress as compared to a corresponding non-genetically modified wild type plant, wherein the genome of said plant comprises a transgene inserted into said genome, said transgene comprising a nucleic acid molecule encoding a ribonucleic acid sequence, which is able to form a double-stranded ribonucleic acid molecule having any one of SEQ ID No: 82, 83, 84 and 84.

18. A genetically modified plant according to claim 13, wherein the plant is a seed or plant part thereof.


SEQUENCE LISTING (95 sequences)

SEQ ID NO: 1 - protein, 24 aa, Vitis sp.
  vWF-A1 consensus peptide of Med25
XXTAAXGPXWXXXXXXYXXXXXRX

SEQ ID NO: 2 - protein, 60 aa, Vitis sp.
  vWF-A2 consensus peptide of Med25
XXXXVXXXXHGXXXXXXVQRXXWTXDXXXFXXWLXXXXFXGGGXXXXXXXEGLXEALXXX

SEQ ID NO: 3 - protein, 18 aa, Vitis sp.
  vWF-A3 consensus peptide of Med25
XCXLXXAXNPXXLXTPVX

SEQ ID NO: 4 - protein, 45 aa, Vitis sp.
  vWF-A4 consensus peptide of Med25
AEXXAXXFXXXXXSLSXXXPKQLPXXXXXXXXXKXNXXXXDXXXX

SEQ ID NO: 5 - protein, 27 aa, Vitis sp.
  ACID consensus peptide 1 of Med25
KYXKXWEGXLSGQRQGQPVXIXXXEXX

SEQ ID NO: 6 - protein, 51 aa, Vitis sp.
  ACID consensus peptide 2 of Med25
LAXXWPXXMQIVRLIXQXHMNNKQYVGKADFLVFRXXXXHGFLXQLQXKKL

SEQ ID NO: 7 - protein, 37 aa, Vitis sp.
  ACID consensus peptide 3 of Med25
CAVIQLPSQTLLLSXXDKAXRLIGMLFPGDMVVFKPQ

SEQ ID NO: 8 - DNA, 2448 bp, Vitis vinifera
  CDS (1)..(2448): Vitis vinifera cDNA encoding med 25 protein
atggcggagaagcagctggtcgtggccgttgaaggcactgccgccatgggtccctattggcaagccgttgtttccgattacctggacaagatcatcaggtacttttgtggtaatgagttggcagggcagaagccctcttcttctaactttgagctctctctggtcatgtttaatgctcatggatcttattgtagttgcttagtacaacgaagtggttggacaagagatgttgatcttttcttgcagtggctgtctgctttaccctttgctggtggtggtttcaatgatgctgcaattgcagaagggcttgctgaagctctgatgatgttctctgttgctgcaaatggaagccaaacccaacaaaatgtggatggacaacgacattgcattctagtagctgcaaataacccttatcccttgcccacaccagtttatcaaccacaaatgcaaaacatggaacagaatgagagcattgaatcacaaacagaaagtcgtctttctgatgcagaggcagttgctaaatcatttgctcagtgctctgtttctttgtcagtcatttgtccaaaacagcttccgaaactaaaatcaatttataatgcgggaaagcgcaatccccgagtagcagatccaccagttgataatgttaaaaaccctcatttcctggttctaatctcagatagtttcatggaggctcgtgctgctttaagtcggcctggactttcaaatatgacagcaaatcagagtcctgtaaaaatggatatagcttcagttcctcaggtttcagcggcccctcctgcttctattccatcagtgaatggtatcatgaaccggccaacaattgctgttggagctgtcccgactgctactgtaaaagttgaaccaagcacggtaacttccatcacatctggacctggttttccacatattccatctgtcccacgtgctgcttctcaaggagttccaagccttcagacttcttcaccatcttctacttctcaggagatgatctcaaatggtgacaatgtccaagatttgaaacctatagtgagtggtatatcacagaccttacgtcctgtggttcccgctgcagcaaatgtaagcattctgaacaacctctctcaagcacggcaagtgatgcattctgcagccctaagtgggggaacttctataggacttcagtcaatgggtgggacttccatggctatgcatatgtcaaatatgatatccagtggaatggcatcatctgtacctgccactcaaactgtgttctcatctgggcagtcagctgtctcatcaataactggatcagggacactagcaggaactgcacaggttgcacaaaactcagctcttggttcgttcacttcagcaacttctaatatgtcagtaaactcaaaccttggcatttctcaaccattgatacctggaatgagccaagggaatcttcctggaggacaaatggtacaaagtggaattggtatgaaccagaacatgatgagtggtcttggtccatcaggtatatcttctggaactggcacaatgattcctactccaggaatgtctcaacaagtgcaaccaggaatgccatctcttggtgtgaacaacaatgcagcagctaatatgcccttaccacagcagacatcaggtgctatgcaaacagcacagtccaaatatgtaaaggtctgggagggaaacttatcggggcagagacaagggcagcctgttttcatcaccagacttgaaggttatagaagcgcatcggcttcagagtcgcttgcagcaaactggcctgcgacgatgcaaatagtacggcttatatcccaggaccacatgaataacaagcagtacgttgggaaagcagattttctagtttttcgagcaatgaaccagcatggatttcttgggcagctgcaggaaaagaagctttgtgcagtcatccagttaccatctcagacattgctgctctctgtttctgacaaagcctgccgtttgataggaatgctttttcctggggatatggttgttttcaagccacagataaccaattcgcagcagcaacaacatcagcaactgcaacaacagcagattccgctgctgcaacaacagcaacagcttgcaccgctccagcaacagcaacagcttccacagctgcaacaacaacaacagcttccccaacttcagcagcagcaacagcttcagcagcagcagcatccccagcttcagcaacagcaacagcttccccagcttcagcaacaacagcagcttccacaactgcagcaacaacaccagctttcccaactgcagccacagcagcagcaccagcccctacaacagcaacagcagccgcaacaaatggttgggacaggaatgggtcaagcttttgttcaaggtccagggcggtcatcacaagggccaccaaacatgcctggaggggcctttctgggctaa

SEQ ID NO: 9 - protein, 815 aa, Vitis vinifera
MAEKQLVVAVEGTAAMGPYWQAVVSDYLDKIIRYFCGNELAGQKPSSSNFELSLVMFNAHGSYCSCLVQRSGWTRDVDLFLQWLSALPFAGGGFNDAAIAEGLAEALMMFSVAANGSQTQQNVDGQRHCILVAANNPYPLPTPVYQPQMQNMEQNESIESQTESRLSDAEAVAKSFAQCSVSLSVICPKQLPKLKSIYNAGKRNPRVADPPVDNVKNPHFLVLISDSFMEARAALSRPGLSNMTANQSPVKMDIASVPQVSAAPPASIPSVNGIMNRPTIAVGAVPTATVKVEPSTVTSITSGPGFPHIPSVPRAASQGVPSLQTSSPSSTSQEMISNGDNVQDLKPIVSGISQTLRPVVPAAANVSILNNLSQARQVMHSAALSGGTSIGLQSMGGTSMAMHMSNMISSGMASSVPATQTVFSSGQSAVSSITGSGTLAGTAQVAQNSALGSFTSATSNMSVNSNLGISQPLIPGMSQGNLPGGQMVQSGIGMNQNMMSGLGPSGISSGTGTMIPTPGMSQQVQPGMPSLGVNNNAAANMPLPQQTSGAMQTAQSKYVKVWEGNLSGQRQGQPVFITRLEGYRSASASESLAANWPATMQIVRLISQDHMNNKQYVGKADFLVFRAMNQHGFLGQLQEKKLCAVIQLPSQTLLLSVSDKACRLIGMLFPGDMVVFKPQITNSQQQQHQQLQQQQIPLLQQQQQLAPLQQQQQLPQLQQQQQLPQLQQQQQLQQQQHPQLQQQQQLPQLQQQQQLPQLQQQHQLSQLQPQQQHQPLQQQQQPQQMVGTGMGQAFVQGPGRSSQGPPNMPGGAFLG

SEQ ID NO: 10 - DNA, 2511 bp, Arabidopsis thaliana
  CDS (1)..(2511): Arabidopsis thaliana cDNA encoding Med 25 protein
atgtcgtcggaggtgaaacagctgatcgtcgttgctgaaggcaccgccgctttgggtccttattggcaaaccatcgtctccgactatctcgagaaaatcatcaggtctttctgtggcagtgagttaaatggagagaggaaccctgtttctactgttgagctatcactggtgatcttcaattctcatggttcatattgtgcttgcttggtacaacggagtggctggacaagggatgttgatattttcttgcattggctttcttccatacaatttggtggtggtggtttcaatgaggttgccacagctgaagggcttgccgaagcattgatgatgttttctcctccttcaggccaagctcaaccaagtaacgatctgaaaagacactgtatcctaatcacagccagcaatcctcacatattgccaacacctgtatatcgtccacgattgcaaaatgtggaacggaatgagaatggtgatgcgcaagctgagagtcgattatcagatgctgagacagtggcttcatattttgctaagtgttctgtttctttgtctgttgtatgtccaaagcagcttccaacaattagagcactatacaatgcgggaaagcccaatcaacaaagtgcggacttgtcaattgacacggctaagaacacattctatcttgtcctgatctcggagaattttgtggaggcatgtgctgccttaagtcattctgctacaaatttgccacagactcagagccctgtgaaagtggacagggccactgttgctccatctattccagtcactgggcaacctccagctcctgtgtcatcagccaatggacctattcagaatcggcaaccagtttctgttggaccagttccaactgctactgtgaaagttgagcctagcaccgtaacttctatggcaccagttcctagttttccccatatcccggctgtagctcggcctgctacacaagcaattccttcgattcaaacatcttcagcatcaccagtttctcaggatatggtcagcaacgccgagaatgcaccagatattaagcctgtggtggtcagtggaatgacgccaccattgcgtactggtcctcctggtggagccaatgtaaatctgcttaataatctttctcaagtccgacaagtcatgagctctgcagctctggcaggtgcagcctcatcggttgggcaaagtgcggttgcaatgcatatgtcaaatatgatatcaacaggaatggctacatctttgcctccttcacaaactgtgttttcaactggacagcagggaattacttcaatggctggttcgggtgcactaatgggatctgcacaaacgggacaaagcccgggtcctaataatgcctttagtcctcaaacaacgtcaaatgtcgcttcaaaccttggtgtttcacaaccaatgcaagggatgaaccaaggaagtcattctggagcaatgatgcaaggtggaatttccatgaaccaaaacatgatgagtggtcttggtcaaggaaatgtctcctctggaacaggtggaatgatgcctactccaggagttggccaacaagcgcaatcaggaatacaacaacttggtggcagtaacagctcagctcctaatatgcagctatcacagccatcatcgggggctatgcagacttcacaatccaaatatgtgaaagtctgggagggaaatttatctgggcaaaggcaagggcagcctgttcttatcaccagacttgagggttaccgaagtgcttctgcctctgattcgttggcagcaaactggccaccaactatgcagattgttcgtctcatatcccaggaccatatgaataacaagcaatatgttggcaaagctgacttccttgtgtttcgggccatgagtcaacatgggttcttaggacaacttcaggataaaaagctttgtgcagtcatccagttgccatcacagacgcttcttctctctgtctctgacaaggcttgccgcttgattggaatgcttttcccaggggatatggttgtgtttaaaccacaaattccaaatcagcaacagcagcagcaacaacaactccaccagcaacaacaacaacaacagcagatccagcagcagcagcaacaacaacaacacctccaacagcaacagatgccacaactccagcaacaacaacaacaacaccagcagcaacagcaacagcagcatcaattgtcacagctccaacatcatcagcagcaacaacaacaacagcagcaacaacagcagcagcatcaattgacacagcttcaacaccatcatcagcagcagcagcaggcgtcgccgctgaatcagatgcagcagcagacttcgccgctcaatcagatgcagcaacagacttcgcctctgaatcagatgcagcagcaacagcagcctcaacagatggtaatgggtggtcaagcttttgcacaagcccctggaagatcacaacaaggtggtggtggagggcagcctaacatgcctggagctggcttcatgggataa

SEQ ID NO: 11 - protein, 836 aa, Arabidopsis thaliana
MSSEVKQLIVVAEGTAALGPYWQTIVSDYLEKIIRSFCGSELNGERNPVSTVELSLVIFNSHGSYCACLVQRSGWTRDVDIFLHWLSSIQFGGGGFNEVATAEGLAEALMMFSPPSGQAQPSNDLKRHCILITASNPHILPTPVYRPRLQNVERNENGDAQAESRLSDAETVASYFAKCSVSLSVVCPKQLPTIRALYNAGKPNQQSADLSIDTAKNTFYLVLISENFVEACAALSHSATNLPQTQSPVKVDRATVAPSIPVTGQPPAPVSSANGPIQNRQPVSVGPVPTATVKVEPSTVTSMAPVPSFPHIPAVARPATQAIPSIQTSSASPVSQDMVSNAENAPDIKPVVVSGMTPPLRTGPPGGANVNLLNNLSQVRQVMSSAALAGAASSVGQSAVAMHMSNMISTGMATSLPPSQTVFSTGQQGITSMAGSGALMGSAQTGQSPGPNNAFSPQTTSNVASNLGVSQPMQGMNQGSHSGAMMQGGISMNQNMMSGLGQGNVSSGTGGMMPTPGVGQQAQSGIQQLGGSNSSAPNMQLSQPSSGAMQTSQSKYVKVWEGNLSGQRQGQPVLITRLEGYRSASASDSLAANWPPTMQIVRLISQDHMNNKQYVGKADFLVFRAMSQHGFLGQLQDKKLCAVIQLPSQTLLLSVSDKACRLIGMLFPGDMVVFKPQIPNQQQQQQQQLHQQQQQQQQIQQQQQQQQHLQQQQMPQLQQQQQQHQQQQQQQHQLSQLQHHQQQQQQQQQQQQQHQLTQLQHHHQQQQQASPLNQMQQQTSPLNQMQQQTSPLNQMQQQQQPQQMVMGGQAFAQAPGRSQQGGGGGQPNMPGAGFMG

SEQ ID NO: 12 - DNA, 2451 bp, Brachypodium distachyon
  CDS (1)..(2451): Brachypodium distachyon cDNA encoding Med 25 protein
atggcggccgagaggcagctggtggttgtcgtggaggggacggcagcgctggggccgtactggcacaccatcgcggccgagtacgtcgagaagatcgttcggagcttttgtgcagctcaactgtcagggcagaagcttgcaggggtaccgcccgagcttgcattagttgtcttccatacccatggaccctacagtgcttttgttgtacaacgcagtggttggacaaaagatatggatacttttctttcatggttatcaggaatatcatttagtggcggaggcttcagtgaagctgctatttgtgaaggtcttgctgaagcattgatgatactccaaggcagtcccagcaacaaccagaatcatcaaaaccatgagttacaaaagcattgtgtacttgttgctgcaagtaatccttaccctctgcctacacctgtctaccgcccttttgttcaaagtggtgatcacaaaaagaacaatgaagtaacaaaggaatcatgtcttgccgatgctgaggctgttgcgatctcattttctcagtgctgtgtatcgttgtcggtggtatctcctaaacagcttccaacactgaaggcaatatacaatgcgggaaagaggaatcctcaagcttctgatccatcagttgatcatgtgaaaaatccgcatttccttgttttgctgtctgagagtttcatggaggctcgaactgctctaagccatccttcacctgggaacctggtcccaaaccaaatcattacaaaaatggacattgctcctgctgctactgtgccaggaccaacttcaaatgccaatccctcagtgaatggatcaatgatgggccggcaaccaactgcaaatattaaagtggagccgacaattataacaccaatggtttctgcacctgcattctcgcatatgacacctatttcaaatgttacttcgcaaggggtatcggcactacaaagttcttcaccatctattatttcacaagaaacaaatgttgcaaatgaaattttgcaagagcataagcctttagtaaaccctatccaacaacaagttcggcctggtggcccagcaaatgtcagcatcctaaataatctgtcacagcatcggcattctttatcaggagctacctcaatgggacctaacatgggggcaacacctatacaagtgcatatgtcaaacatgatatcaagtggcatgacgtcaactccctctgtcatatcttctatgtctggaacagtacagccaactggtgcgcaacagttggtacaaaacacagctcttggttcttttggatcgaacaattctactgtatctggcaattcaaatgttgctgtatcatcttctttggccaacatacaaaacaacattgctactgcacaatcagttcctcctatggcacaaggtggtctgatgtctggttcacatggttcacaatctggacaaggtggaattggtacgaaccaaaatatgataagcagccttggacctacagctatctcttctgcacctgcaatgatgccaacaccagggatgacccaacaggcaggagtaaattctcttagtgtgaataacagttctgctatgaatatgcctatgccgcagcaacctaatggccagcaacagtccaagtatgtcaaaatttgggagggaactttgtctggtcaaaggcaaggacagcctgtatttatttgtaaacttgaaggttacaggagtgggacagcatctgacacacttgctgcagactggccagaaacaatgcagattgtgcgcctcatagcccaggagcatatgaacaataagcagtatgttggcaaggcagactttctagtattccggacattgaatcagcatgggttccttggacaactgcaagagaagaaactgtgtgccgtgattcagttaccttcacaaactttgctgttgtcagtttctgataaagctgggcgactcattggaatgctgttcccaggggatatggtagtatttaaaccacaggtctcaacccagcagccacagatgcagcaacagcagcagctacaacagcagcaccacccactacaccagcagcagctacaacaattacagcagcagcaactacaacagcaacacatgcaaatgcagccgcaaggccaacaacttcagcagcagcagcagattcagcaacaaatgcaacagcaacagcaacaacaacaacagatgcagcaaatgcaacaccaacagcagcagcagcagcaaattcaacagcagcaacagatgcagcagcaacagcaacagatgcagcaaatgcagcagcagcagcctcagcaactccagcagcagccgcagatggtgggtccagggatggggcagcagcaattcatgcaggggcatggtcgggcggtgcagatgatgcaaggaaagatcgcgccacagggaccaggcagcatgtctgggggaggctacctatcttga

SEQ ID NO: 13 - protein, 816 aa, Brachypodium distachyon
MAAERQLVVVVEGTAALGPYWHTIAAEYVEKIVRSFCAAQLSGQKLAGVPPELALVVFHTHGPYSAFVVQRSGWTKDMDTFLSWLSGISFSGGGFSEAAICEGLAEALMILQGSPSNNQNHQNHELQKHCVLVAASNPYPLPTPVYRPFVQSGDHKKNNEVTKESCLADAEAVAISFSQCCVSLSVVSPKQLPTLKAIYNAGKRNPQASDPSVDHVKNPHFLVLLSESFMEARTALSHPSPGNLVPNQIITKMDIAPAATVPGPTSNANPSVNGSMMGRQPTANIKVEPTIITPMVSAPAFSHMTPISNVTSQGVSALQSSSPSIISQETNVANEILQEHKPLVNPIQQQVRPGGPANVSILNNLSQHRHSLSGATSMGPNMGATPIQVHMSNMISSGMTSTPSVISSMSGTVQPTGAQQLVQNTALGSFGSNNSTVSGNSNVAVSSSLANIQNNIATAQSVPPMAQGGLMSGSHGSQSGQGGIGTNQNMISSLGPTAISSAPAMMPTPGMTQQAGVNSLSVNNSSAMNMPMPQQPNGQQQSKYVKIWEGTLSGQRQGQPVFICKLEGYRSGTASDTLAADWPETMQIVRLIAQEHMNNKQYVGKADFLVFRTLNQHGFLGQLQEKKLCAVIQLPSQTLLLSVSDKAGRLIGMLFPGDMVVFKPQVSTQQPQMQQQQQLQQQHHPLHQQQLQQLQQQQLQQQHMQMQPQGQQLQQQQQIQQQMQQQQQQQQQMQQMQHQQQQQQQIQQQQQMQQQQQQMQQMQQQQPQQLQQQPQMVGPGMGQQQFMQGHGRAVQMMQGKIAPQGPGSMSGGGYLS

SEQ ID NO: 14 - DNA, 2538 bp, Carica papaya
  CDS (1)..(2538): Carica papaya cDNA encoding Med25 protein
atggctgagaaaaaactagttgtcattattgaaggaactgcagcaatgggtcccttctggaagtctattctctctgactatctggaagagattgttaggtctgattttaaactaatatcacctgcttcacttctaaggtgtttctgtggtaatgaattaacggggcagaagacttcttcttccaatgtggagctctcactggttacctttaattgtcatggatcatattctggttgccttgttcaacggagtggctggaccaaagatgttgatactttcttgctgtggctcacagctcttcctttcgctggtggtggtttggatgatgctgccattgccgaagggcttgctgaggctctgatgatgtttgacatcaatttaaatgggagccaagctcaacaaaatgttgacttgcaaagacattgtatccttattgctgcaagcaatccttatgctttgccaacaccagtctatcgaccaaaattgcagaatttggatcagaatgaaaatgttgaaggtcagacagaaagtcgtctatcagatgctgagtcactggctaaatcatttccaatgtgctctgtgtctttatcagtcatatgtccgaaacagcttccaaaacttagagcaatctacaattcggggaagcgcaattcccgagctgcagatccacaggttgacaatgttaagaatcctcagtttcttgttctgatatcagagaactttatggaggcacgtgctgctttaagccgtccgggatcaaccttggcaaatcagagccctgtgaaaatggacatagcttctgctacttcagttacagggcagccccctgcttctgttccatcagtgaatggatctgtcatgaaccggcaaccaatttctgtcggaaatgttccttctaccactgtaaaagttgagccaagtactgtgacttccatggtaactgggcctgcttatccacatattccatctgttgctcgtcctgtttctcaaggaatcccgaatttgcaaacttcttcaccattgtctacttctcaagatatgatcataaataatgataacgtgcaagaagttaaacctattgtcagttctatgacacagcccttaaggccaatggctcctggtgctgcaaatgtgagcatactgaataatctctctcaagtacgacaagtgatgaactctgctgcccttaccgggggaacttcagcaggacttcaatcaatgggccaaactcctgttgccatgcatatgtcaaatatgatatctagtggaatggcatcttctgtgcctgctgcccaaaatgtgttctcttcggggcaatcagttattacttcaataacggggtcagggaccattactggcacttcacaggttgcacaaaactcaggtcttaatacattcacttctgcaacgcctagtgtttctggaaatgcaaatcttggaatttcacaatcaatggctactattcaaggaggtgctagtgttgcccaagctgtgactggcatgaaccaaggaagtcactcaggagcacaaatggtacaaagtggaattggaatgaaccaaaacacgatgactggagttggtccatcaagtgtttcatctgggactggcgcaatgattcccacttcaggaatgtctcaacaagtccagcctggcatgcagccacttggtgtgaacaacagttcagcagtcaacatgccattgtcacaacatgcatcaagttctttggcatcctcatcacagtccaaatatgtcaaggtttgggagggtaatttatctggacagagacaagggcagcctgtctttatcaccagactggagggttataggagctcatcagcttctgacacgcttgcagcaaattggccaccaaccatgcaaatagttcgtctcatatctcaggatcatatgaacaacaagcaatatgttgggaaagcagatttcctggtttttcgagctatgaatcagcatggctttcttggacagctgcaagaaaagaagctttgtgcagtcatccaattaccatctcagacgctgttgctttctgtttctgacaaagcctgccgcctgataggaatgcttttccctggggatatggttgtctttaaaccacaattaccaagtcagcagcagcaacaacagcagcagcagatgcaacaacagcaacagatgcaacaacagcagcatccacagctgcaacagcagcagcttcctcagcttcaacaacagcagcaacatccgcagcagcagcagcagcaacctcagcaacagccactcccgcagcttcagcaacaacagcttccgcagctgcaacagcagcagcagcaacctctctctcagctccaacaacagcaaccattgccacagctgcagcaacaacaacagcagcttccccaactgcagcaacagcagcaactaccacggcagcagcaaattgttgggtccagcatggctccagtctatccaacgcgtgcgcagttgcttgctcagacttcatcacaagggccgcctgacatccctggggcaggctttatgggttag

SEQ ID NO: 15 - protein, 845 aa, Carica papaya
MAEKKLVVIIEGTAAMGPFWKSILSDYLEEIVRSDFKLISPASLLRCFCGNELTGQKTSSSNVELSLVTFNCHGSYSGCLVQRSGWTKDVDTFLLWLTALPFAGGGLDDAAIAEGLAEALMMFDINLNGSQAQQNVDLQRHCILIAASNPYALPTPVYRPKLQNLDQNENVEGQTESRLSDAESLAKSFPMCSVSLSVICPKQLPKLRAIYNSGKRNSRAADPQVDNVKNPQFLVLISENFMEARAALSRPGSTLANQSPVKMDIASATSVTGQPPASVPSVNGSVMNRQPISVGNVPSTTVKVEPSTVTSMVTGPAYPHIPSVARPVSQGIPNLQTSSPLSTSQDMIINNDNVQEVKPIVSSMTQPLRPMAPGAANVSILNNLSQVRQVMNSAALTGGTSAGLQSMGQTPVAMHMSNMISSGMASSVPAAQNVFSSGQSVITSITGSGTITGTSQVAQNSGLNTFTSATPSVSGNANLGISQSMATIQGGASVAQAVTGMNQGSHSGAQMVQSGIGMNQNTMTGVGPSSVSSGTGAMIPTSGMSQQVQPGMQPLGVNNSSAVNMPLSQHASSSLASSSQSKYVKVWEGNLSGQRQGQPVFITRLEGYRSSSASDTLAANWPPTMQIVRLISQDHMNNKQYVGKADFLVFRAMNQHGFLGQLQEKKLCAVIQLPSQTLLLSVSDKACRLIGMLFPGDMVVFKPQLPSQQQQQQQQQMQQQQQMQQQQHPQLQQQQLPQLQQQQQHPQQQQQQPQQQPLPQLQQQQLPQLQQQQQQPLSQLQQQQPLPQLQQQQQQLPQLQQQQQLPRQQQIVGSSMAPVYPTRAQLLAQTSSQGPPDIPGAGFMG

SEQ ID NO: 16 - DNA, 2595 bp, Cucumis sativus
  CDS (1)..(2595): Cucumis sativus cDNA encoding Med25 protein
atggcagataagcagttgatagtggtcgttgaaggtaccgcagccatggggcccttctggcaaaccgtcgtttcggattacattgaaaagatcgttagatctttctgtggaaatgaattaactgggcagaaaccaccaacctctaatgttgagttctctctggtcacatttcacactcatggatcatattgtggatgtctagtacagcggactgggtggacaagagatgtcgatatttttatacaatggctttcagctattcccttttctggtggtggtttcagtgacgctgcaatagctgaagggctggctgaagcactaatgatgttccctactcagccaaatggtggccaaaatcaacaaactatggatatgcagaagcactgtattcttgttgctgcaagtaatccttatcccctgccaacgccagtatatagaccagcagtacaaaatttggagcaacacgacaatgttgaaccagggagctctcaatcatatgctgaaacagttgctaaatcatttcctcagtgcttcatatctctgtcagttatttgtccgaaacagcttccaaagctcaaagcaatttacactgctggaaagcgcaacccacgggcagcagatccacctatcgataatgttaaaagcccctcataccttgttttgatctctgaaaattttgtggaggctcgtgcagctttaagtcgtcctggaattactagtttacctgcaaaccagagtcctgtgaaaatggacatatcttctgttgtgcctgttactggaccacctcctaccactactccatcagtaaatggacctatcattaaccggcagcccgtttctgttcctaatggccctactgctactgtgaaagttgaaccaaatactgttacttctatgacaaatggatctggatatccaccacatatgccatcggttgttcgtgcagcttcacagggagtcccgagcttgcaaacttcttcaccactttcttctcaggagatgatcacaaataatgagaacacacaagatcttaaacctctagtgactggtgttccacagccagtacggtccttgggtcctgccaatgttagcattctgaataatatttctcaagcacgagtgatgagcactgctgcgttaaatggaggaacttccataggactcccatctatgggtcaaaccccaatagccatgcatgtatccaatatgatatccagtggaatgggatcatctgtgcctgctgcacaaaatgtattttcatctggtcaatcgggcatgacatcaataaatggatcttcaacactttcacaggttgcaccaaactcaggtataagttcacttacttcaggaaacaacaatatttctggaaatccaaatattgccacttcacaagcagtaggaaatctccaaggtagtgttagtgtgagtcaatcagtacctgggattagccaaggaaatcttgctgggacacaagtggtacaaagtggaattggcgtgagccaaaatgttatgagcaacctaacccaacctggtgtttcttctggaaatggtacgatgattcctactccaggaatgcctcaacaagttcaaaatgggatgcattcacttggcatgaacaatgcagcagctaatatgccattaccacagcattcagcaggtgctttgcaacaacaagcacaatccaaatacgtgaaattttgggagggaaatctatctggtcagagacaaggccagcctgtttttattacaagaatggaagcttttgtagtgattagaaaattacgttttctcttacacgatgtcaagcttgctgcaaactggccacagacaatgcaaatagttcggcttatctcacaagatcacatgaataataagcaatatgttggaaaagctgattttcttgttttccgagctatgaatcagcatggatttcttggtcagcttcaagaaaagaaactttgtgcagtcatccagttaccatctcagactttactgctgtccgtttctgataaagcttgccgcttgatagggatgctttttcctggggatatggttgtgtttaagcctcaaatatccagtcaacagcaacaacagcatcaaatgcaacaacaacaaatgcaacagcaacttcaacagcaacagcaacatcaactttctcagctgcaacagcagcatcaacttcctcagatgcaacaacagcagcagcagcagcagcaacagccacagctttctcatatgcaacaacagcaacagcaacagcaacagcagcagcctcttcctcaaatccaacagcagcagcagtcgcagcttcctcaaatacagcagcagcagcagcaacagcagcaaccacaattgtcacagcttcaaaaccagcagcaaccacaactgtcacagcttcaaagccagcagcaaccacaactgtcacagcttcaaagccagcagcagattccacaaatgcagcaacaacagcaacaaatggttgcgggtggaatcaatcaatcttatgtccaaggagggcgatcacagttagtttcacagggacaagtctcttcccagggtcaatctaacatgtctgctggaaactttatgggttga

SEQ ID NO: 17 - protein, 864 aa, Cucumis sativus
MADKQLIVVVEGTAAMGPFWQTVVSDYIEKIVRSFCGNELTGQKPPTSNVEFSLVTFHTHGSYCGCLVQRTGWTRDVDIFIQWLSAIPFSGGGFSDAAIAEGLAEALMMFPTQPNGGQNQQTMDMQKHCILVAASNPYPLPTPVYRPAVQNLEQHDNVEPGSSQSYAETVAKSFPQCFISLSVICPKQLPKLKAIYTAGKRNPRAADPPIDNVKSPSYLVLISENFVEARAALSRPGITSLPANQSPVKMDISSVVPVTGPPPTTTPSVNGPIINRQPVSVPNGPTATVKVEPNTVTSMTNGSGYPPHMPSVVRAASQGVPSLQTSSPLSSQEMITNNENTQDLKPLVTGVPQPVRSLGPANVSILNNISQARVMSTAALNGGTSIGLPSMGQTPIAMHVSNMISSGMGSSVPAAQNVFSSGQSGMTSINGSSTLSQVAPNSGISSLTSGNNNISGNPNIATSQAVGNLQGSVSVSQSVPGISQGNLAGTQVVQSGIGVSQNVMSNLTQPGVSSGNGTMIPTPGMPQQVQNGMHSLGMNNAAANMPLPQHSAGALQQQAQSKYVKFWEGNLSGQRQGQPVFITRMEAFVVIRKLRFLLHDVKLAANWPQTMQIVRLISQDHMNNKQYVGKADFLVFRAMNQHGFLGQLQEKKLCAVIQLPSQTLLLSVSDKACRLIGMLFPGDMVVFKPQISSQQQQQHQMQQQQMQQQLQQQQQHQLSQLQQQHQLPQMQQQQQQQQQQPQLSHMQQQQQQQQQQQPLPQIQQQQQSQLPQIQQQQQQQQQPQLSQLQNQQQPQLSQLQSQQQPQLSQLQSQQQIPQMQQQQQQMVAGGINQSYVQGGRSQLVSQGQVSSQGQSNMSAGNFMG

SEQ ID NO: 18 - DNA, 2109 bp, Eucalyptus grandis
  CDS (1)..(2109): Eucalyptus grandis cDNA encoding Med25 protein
atgtcgacgacggagagatcgctgatcgtggccgtcgacggcaccgctgccatggggccttactggaaaaccatcgtttccgattacctcgagaagatcgtcaggtccgtttgcgcttctaactgtgatctctctctggtcatgttcaattctcatggaagtttgtctgcttgcatggttcagcgaagtgcttggacaaaagatgtcagctgtttcctagaatggctctcagcaatgtcttttagtggtggtggttttaatgacgctgctattgcggaaggcctttctgaagctttgatgatgttcccccttcctctgaatggaaacctacctccgcaaagtctagatggcaaaaggcactgtgttcttgttgctgcaaccaatccttatcccttgtcaactccagtttatcgaccgacttttcaactggaagatgagcatgttgaaacttactctggaaaacctttgtgtgatgctgagggtgttgcaaagtttttccctcagtgctgtgtttctctctcagttatttgtccaaagcagcttccaaaacttcgggcaatctacaatgctggaaaacgcaatccacgtgcagcagatccacctgttgataatggaaagaatacccatttccttgtattaattgcggagaatttccttgaggcacggacagctttgagtcgtcctggaacagctagtttgccttcaaatcagagtcctgtcaagatggacacaactccacctccagttaattcagtcacggcacaacctcctgcatctgttccagaaccaactacagtgacttctatggcagctggccccaacttttctcacattccgtctgttcagcgtcctactactcaaggagtctctgccttgcagacttcctcaccttcttctgtatctcaagacatggtaacaaacaatgaaagcgtacaggatttgaagccagtggtgagtgctggcattccacaaacattgcgacctgtagttcctgctaattcaaacatactaaacaacctctctcaagtacgacaagtggtcaattctgcaacattgactggagggacttcaatagggctccaatctatgggtcaaaatccaatggccatgcatatgtcaaatatgatttctagtgggatgcagtctactgtgcctgcagctcaaacggggtattcttcaggccaatcgggcatcacctctgtgacgggtcctggcactctctcggggaccataccatctgcacaaaatccaggcctcggttcatttgcttcagcaactagcaacattactggaaattcaaacattggaatgtcccaaccaactggcaatcttcaagttggtcctggtatgggtcaaacagctccaggcatgagccaaggaagcctatcaggatcacaaatggtgcagaatggagttggcatgaatcctaatatgatgagttccctgggtccaacagctccctcttccggaagtggcacaatgatgcccactccaggaatgccttcacaaggacaatctggcatgtcatcaattaatggtaacagcaatccaggtccaggtgcaagtatgccattgtcacagcaatcgtcaagtgctttgcagagtgcacagtccaaatatgtgaaagtttgggagggaaatttatctgggcaaagacaaggacagcccgtcttaatcaccagattggaggggtataggagtgcatcagcttctgagacgcttgcagcaaactggccaccaaccatgcagatagttcgtctaatatctcaggaccacatgaataacaagcaatacgttgggaaagcagatttcctggtttttcgggcaatgaatcagcatggttttcttgggcagctccaagagaaaaaactttgtgcagtcattcaattaccgtctcagacgctgctgctctctgtgtcagacaaggcatgccgcttgattggaatgcttttccctggggatatggttgtattcaagccgcaaattccgagcgggtccggaatgggacaaggctatgtccaaggtccagggcgttcacagttgatgccacagggacccgtttcatcacaagggctgcccaacatgcctggcgggggattcatgagctaa

SEQ ID NO: 19 - protein, 702 aa, Eucalyptus grandis
MSTTERSLIVAVDGTAAMGPYWKTIVSDYLEKIVRSVCASNCDLSLVMFNSHGSLSACMVQRSAWTKDVSCFLEWLSAMSFSGGGFNDAAIAEGLSEALMMFPLPLNGNLPPQSLDGKRHCVLVAATNPYPLSTPVYRPTFQLEDEHVETYSGKPLCDAEGVAKFFPQCCVSLSVICPKQLPKLRAIYNAGKRNPRAADPPVDNGKNTHFLVLIAENFLEARTALSRPGTASLPSNQSPVKMDTTPPPVNSVTAQPPASVPEPTTVTSMAAGPNFSHIPSVQRPTTQGVSALQTSSPSSVSQDMVTNNESVQDLKPVVSAGIPQTLRPVVPANSNILNNLSQVRQVVNSATLTGGTSIGLQSMGQNPMAMHMSNMISSGMQSTVPAAQTGYSSGQSGITSVTGPGTLSGTIPSAQNPGLGSFASATSNITGNSNIGMSQPTGNLQVGPGMGQTAPGMSQGSLSGSQMVQNGVGMNPNMMSSLGPTAPSSGSGTMMPTPGMPSQGQSGMSSINGNSNPGPGASMPLSQQSSSALQSAQSKYVKVWEGNLSGQRQGQPVLITRLEGYRSASASETLAANWPPTMQIVRLISQDHMNNKQYVGKADFLVFRAMNQHGFLGQLQEKKLCAVIQLPSQTLLLSVSDKACRLIGMLFPGDMVVFKPQIPSGSGMGQGYVQGPGRSQLMPQGPVSSQGLPNMPGGGFMS

SEQ ID NO: 20 - DNA, 2634 bp, Glycine max
  CDS (1)..(2634): Glycine max cDNA encoding Med25 protein
atggcggagaaacagctcatcgttgcagtcgaaagcaccgctgctatgggaccctattggaacaccattctaatggattatctcgataagatgatcaggtgtttcggtggaaatgactcaactggacagaaattttctgcttccaatgtggagtttgctctagtcacctataatactcacggatgttattctagttgccttgtgcaacggtctggttggaccagagacccagatgttttcttctcgtggctatcatctataccctttaatggtggtggttttaatgatgctgcaattgctgaagggctttctgaagctctgatgatgctctggaattctcaaagcggagcccctaatcagcagagtgtggatatgcacaagcattgtatccttgttgcagcaagtaatccctatccgttgcagacaccagtttatgttccgcgaccacagaacctagagcagagtgaaaccattgactcagactcagggagccatttatatgatgctgaagctgtagctaaagcatttcctcagttttctatttctctgtcagtcatctgcccaaaacaacttcccaaaattaaatccatctacaatgcgggaaaacgaaataatagagcagctgatccaccacttgaggctaaaacgcctcattttcttattttaatatcagaaggtttcagggaagcccggggtgctttgagtcgttctgggatcacaagtttgccttcaaatcaaagtcctgttaaagtagatgctgtttctgtaacaccagttacaggggcacccccaacttctatgccagttaatggatctattccaaatcggcaaccagttcctgctgggaatgtggcccctgctactgtaaaagtggagccagtgcctgtaacctccatggtatctggaccggcttttcctcataattcgtcagttcctcgtgctactagtactagtcaaggagttccaagcttgcagacgtcttctccatcttctgtttctcaagatattataacaaataatgaaactgcacaggatacaaagcctacagtgtcaatgctgccccttcgtccagtgaatcctgttcaagcaaatgtgaacattttgaacaatctctctcaagcacgacaggtgatgaactctgctgccttaagtggtggaacctctatgggacttccgtcaatgggtcagactcctgttgccatgcacatgtcaaacatgatatctagtgggatgacatcttctgtacctgcggctcaaaatgtattttcatctggacagtcaggaataacatcaatgactagttctggacctcttactgttcctgcacaggttggacaaaattcaggtctcggttcattaacttcgaacacttctaatttgtcttcaagttcgaacattgggatttcacaaccattgggtaatcttcaaggggttgttagtattggacaacaggtgtccggtatgagccaaggaaacctttcaggggcccaaatggtgcaaggtggagttagcatgaaccaaaatgtaatgagtggtcttggtcaatcagttgtttcttctgggactggcacgatgattcctactccaggaatgtctcaaccagtacaatcagttatgcaaccacttgtgaacaatgccgcagctaatatgcctttgtcacaacagacatcaggtggtatgcaatcggcacaatccaaatatgtgaaggtctgggagggaagcttgtctggacaaagacaaggacagccagtatttatcaccaaacttgaaggttacaggaattcttccgcctctgagacacttgcagcaaactggccccctgtaatgcaaatagttcggcttatatctcaggaccacatgaataacaaacaatatgtaggaaaggcagatttcctagtttttcgggcaatgaacccacatggatttcttggtcagctgcaggaaaagaagctgtgtgcagttattcaattaccatcacagacgttgctactatctgtttctgacaaagccttccgcttgattgggatgctttttcctggggatatggttgtatttaagccgcaattgtcgagtcagcagcagcaacaaatgcagcaacaacatcagcagatgcaaaaccaacagcagcatcttccgcagttgcaacaacagcagcagctacctcacatgcaacaacagcaacttcctcagctccaacaacagcagcaacaacttcctcagctgcagcagcagcagcagcaacttcctcagctccaacaacagcagcaacaacttcctcagctgcagcagcagcagcaacaacttcctcagctgcagcagcagcagcaacaacttcctcagctgcaacagcagcagcaacaggtccagcagcagcagcttgcgcagctgcaacagcagcagcttccgcaaatccagcaacagcagcagctacctcaacttcaacaactcccacaactacagcaacagcagcagcttccgcagttacagcaactgcagccgcagcagcaacagatggttgggtcaggaatgggtcaagcctatgttcaaggccccgggcggtcacagctggtttcgcagggacaagtttcatcacagggagcaacaaacataggtggagggggcttcatgagttaa

SEQ ID NO: 21 - protein, 877 aa, Glycine max
MAEKQLIVAVESTAAMGPYWNTILMDYLDKMIRCFGGNDSTGQKFSASNVEFALVTYNTHGCYSSCLVQRSGWTRDPDVFFSWLSSIPFNGGGFNDAAIAEGLSEALMMLWNSQSGAPNQQSVDMHKHCILVAASNPYPLQTPVYVPRPQNLEQSETIDSDSGSHLYDAEAVAKAFPQFSISLSVICPKQLPKIKSIYNAGKRNNRAADPPLEAKTPHFLILISEGFREARGALSRSGITSLPSNQSPVKVDAVSVTPVTGAPPTSMPVNGSIPNRQPVPAGNVAPATVKVEPVPVTSMVSGPAFPHNSSVPRATSTSQGVPSLQTSSPSSVSQDIITNNETAQDTKPTVSMLPLRPVNPVQANVNILNNLSQARQVMNSAALSGGTSMGLPSMGQTPVAMHMSNMISSGMTSSVPAAQNVFSSGQSGITSMTSSGPLTVPAQVGQNSGLGSLTSNTSNLSSSSNIGISQPLGNLQGVVSIGQQVSGMSQGNLSGAQMVQGGVSMNQNVMSGLGQSVVSSGTGTMIPTPGMSQPVQSVMQPLVNNAAANMPLSQQTSGGMQSAQSKYVKVWEGSLSGQRQGQPVFITKLEGYRNSSASETLAANWPPVMQIVRLISQDHMNNKQYVGKADFLVFRAMNPHGFLGQLQEKKLCAVIQLPSQTLLLSVSDKAFRLIGMLFPGDMVVFKPQLSSQQQQQMQQQHQQMQNQQQHLPQLQQQQQLPHMQQQQLPQLQQQQQQLPQLQQQQQQLPQLQQQQQQLPQLQQQQQQLPQLQQQQQQLPQLQQQQQQVQQQQLAQLQQQQLPQIQQQQQLPQLQQLPQLQQQQQLPQLQQLQPQQQQMVGSGMGQAYVQGPGRSQLVSQGQVSSQGATNIGGGGFMS

SEQ ID NO: 22 - DNA, 2463 bp, Medicago truncatula
  CDS (1)..(2463): Medicago trunculata cDNA encoding Med25 protein
atggcggagaaacagcttatcgttgctgttgaaaccactgcagctatgggaccttattgggatactcttctaatggattatcttgaaaaaatcgtcaggtgccttggtgggaacgagtcgactgggcagaagccttctggttccaacgtcgagttttccctagtcacctataatactcatgggtgttattctggtatcctcgtgcaacgtactggctggacaagagacccagatgttttcttacagtggctagaatctataccattttctggtggtggttttaatgacgcagctattgctgaagggcttgctgaagctctgatgatgttccccccttctcaaagtggaggcctaaatcagcagaatgtggatacgaacatgcactgtatccttgtggcagccagtaatccttacccattgcagacaccggtatatgttccgcaactgcagagcctagagaagactgaatccattgactcaaaccaagtgaaccagttatatgatgctgaagctgttgctaaagcgtttccgcagtttaatatttctctatcggtcgtctgcccaaaacaacttcccaaaattaaagccatctacaatgcgggaaagcgaaataatagagctgctgatccaccggttgatgctaagaccactcattttcttattttaatatcagaaggttttagggaagcccgcagtgccttgagtcgacctggaactaacatgccttcaaatcaaagtcctgtaaaagtagacgctgtatctgccacaccagttacgggggcacccccatcttcattgccatcagtgaatggatctattccgaaccggcaaccaatacctgctgggaatgtgactcctgctactgtaaaagtggagcaagtgcctgtaacttctggaccagctttttcacataatccatcagttccgcgtgctactggtacgggtctaggagttccaagcttgcagacatcttctccatcctctgtttctcaagatattatgacaagtaatgaaaatgcaatggatacaaagcctattgtgtcaatgttgcaaccaatacgtcctgtgaatccagctcaagcgaatgtgaacattttgaataatctctcacaagcacgtcaggtaatggctttaagtggcggaacttctatgggacttcaatcaatgggccagactcctgttgccatgcacatgtcaaacatgatatccagtgggacgacatcttctgggcctacaggtcaaaatgtattttcatctggaccgtcagtaataactagttctggatctcttactgcttctgcacaggttggacaaaactcaggtctttcttcattaacatcagccacttctaactcaagtccaaatactgggatttcacaaccaatagctaatctccaaggaggtgttagtatgggacaacaggttcccactatgaacccaggaaacctttcaggggcccaaatggtgcaaggcggagttaacatgaaccaaaatgtaataaatggcctcggtcaatcaggtgtctcttctggaactggagcaatgattcctactccaggaatacctcaacaggtacaatcaggtatgcaacctcttgtgaataatgcagctgctgctaatatgactttgacgcaacagacagcttcttcacaatccaaatatataaaggtttgggagggaagcttatcaggacaaagacaaggacagccggtatttatcaccaaactcgaaggttacaggagttcttctgcaagtgagacacttgcagcgaactggcctcctgtaatgcaaatagttcggcttatatctcaggatcacatgaataacaagcaatatgtaggaaaggcagattttctagtatttcgggcaatgaacccgcatggttttcttggtcagctgcaggaaaagaagctgtgtgcagttattcaattgccatcacagacgttgctactatctgtgtcagacaaagcctgccgcttgatcgggatgctttttcctggggacatggttgtatttaagccacaattgtctggtcagcagcagcagcaacagatgcaacagcaacaaatgcaacaacaccagcagatgcaatcgcaacagcagcatcttccacaattgcagcaacagatgcaacaacaacaacaacagcaacagcttccacagctgcaacagaatcagcagctttcccagattcaacagcagattccacaactccagcaacagcagcagcaactccctcaacttcaacagcagcagctttctcagctgcagcaacagcagcaacagcttccgcagttacagcaactgcaacatcaacagcttcctcagcagcaacaaatggttggtgctggaatgggtcaaacctatgttcaaggtcctggggggcgctcacaaatggtttctcagggacaagtttcatcacagggagctacaaacattggtggagggaatttcatgagttag

SEQ ID NO: 23 - protein, 820 aa, Medicago truncatula
MAEKQLIVAVETTAAMGPYWDTLLMDYLEKIVRCLGGNESTGQKPSGSNVEFSLVTYNTHGCYSGILVQRTGWTRDPDVFLQWLESIPFSGGGFNDAAIAEGLAEALMMFPPSQSGGLNQQNVDTNMHCILVAASNPYPLQTPVYVPQLQSLEKTESIDSNQVNQLYDAEAVAKAFPQFNISLSVVCPKQLPKIKAIYNAGKRNNRAADPPVDAKTTHFLILISEGFREARSALSRPGTNMPSNQSPVKVDAVSATPVTGAPPSSLPSVNGSIPNRQPIPAGNVTPATVKVEQVPVTSGPAFSHNPSVPRATGTGLGVPSLQTSSPSSVSQDIMTSNENAMDTKPIVSMLQPIRPVNPAQANVNILNNLSQARQVMALSGGTSMGLQSMGQTPVAMHMSNMISSGTTSSGPTGQNVFSSGPSVITSSGSLTASAQVGQNSGLSSLTSATSNSSPNTGISQPIANLQGGVSMGQQVPTMNPGNLSGAQMVQGGVNMNQNVINGLGQSGVSSGTGAMIPTPGIPQQVQSGMQPLVNNAAAANMTLTQQTASSQSKYIKVWEGSLSGQRQGQPVFITKLEGYRSSSASETLAANWPPVMQIVRLISQDHMNNKQYVGKADFLVFRAMNPHGFLGQLQEKKLCAVIQLPSQTLLLSVSDKACRLIGMLFPGDMVVFKPQLSGQQQQQQMQQQQMQQHQQMQSQQQHLPQLQQQMQQQQQQQQLPQLQQNQQLSQIQQQIPQLQQQQQQLPQLQQQQLSQLQQQQQQLPQLQQLQHQQLPQQQQMVGAGMGQTYVQGPGGRSQMVSQGQVSSQGATNIGGGNFMS

SEQ ID NO: 24 - DNA, 2334 bp, Mimulus guttatus
  CDS (1)..(2334): Mimulus guttatus cDNA encoding Med25 protein
atggtcccgaaacagctaatcgtcgccgtcgagggcacggcggccatcgggcctttctggcaaacaatcgtctccgattacctcgacaaaatcatacgatccttttgtggaaacgagccgacgggtcagaagccggctactacccattttgagctctccctagtcatgttcaatgctcacggttcttacagtgcttgtcttgtgcaacggagtggttggacaaaagacatggatatattctttcaatggctttcggctatcccctttgccggtggtggttttaacgacgctgcgatcgccgaagggctatcagaagctttaatgatgttttcttccccaaagggcaaccaaaatcaaaatgtggaaggacaaagacactgcattcttattgctgcaagcaacccttatccattacccacaccagtatttcgaccacaaatccaaaatccagaaaagaacgacggtgttgaatcgcaaccagataatcgtctgtctgatgcagagacattagcaaaatcttttgctcagtgtgctgtttctctatccgttatttgtccgaagcaacttccaaaactcagaggaatctttaatgcgggaaaacgcaatccaagagctccagatccgcctgtagacaataataagaatcctcatttccttgttctcatctcggagaattttatggaggctcgttcttgtttaagtaggtctggaattacaagtctgccaccaaatcaaatccccgtcaagatggatgtcacacctgctcctcctcccatttctgggccacctccagtttctatcccggcagtcaatggatctctcttggcgcggcaaccagtacctgctgcaaacatccctcctgcaattgtaaaattggagcctcccaccattgcaccctccatgacggggcccacatttccacacatctcatctattcctcgagccgttccacaaaccgttccaaccatgcaaacttcttctcctatatcaactcctcaggaagttatcccgatcaacgagaacattccggaagtgaaaccgttggttgccagctcagcacccccgatacgtccagctggcggcgccgcggcgaatgtgagaattttgaacgatatggctcaagcccgccaagctctagccgcagcagccgccggaggaggaggaggagcttccattgggatcccttctatgggcggcggcaccaccatgctctcgaatatgatatccagcggaatgatgacatcatcctccatgtcgtcgggcccgtctatcgtcacctcaatttctgggccgttgccgatgtcagcgaatggacaggtggcgcagaacaattctggaaattctggcattgccatgtcatcgcagccattgagtggcctacaagggaacgtgggtcccggccagacggggaatattccggtggtcccgcagatggtgcagacggggatgggtatgaatgctaatatgatgagtggcggtggtggtgcttcgggaatgtctcagcagggacaacctcctggtggtatgaatagcaataatgttgtgaatactttgaatcagcaaacatcggctacaatgcagtcggcgcagtcgaaatacgttaaagtctgggaggggaatttatcgggacagagacaaggccagcctgttttcatcaccagactggaggggtacaggagtgcctcagcttccgagacgcttgctgcaaactggccatcaacaatgcaaatagttcgcttaatatcccaagatcacatgaataacaagcaatatgtcggcaaggccgattttctcgtttttcgggcaatgaatcagcacggctttcttggacagctccaagaaaagaagctttgtgcagttatacagttaccgtcacagacattgctcctctcggtttcagacaaggcccatcgtttgatcggaatgcttttccccggggatatggttgtatttaagccacaaatacctaataataataacaataataataataatcagcagcagcagcagcaaatgcaagtgcaattacagcagcagcaaatgcaagtgcaattgcaacagcagcaaatacaacaacagcaacaaccaataatgcagattcagcagcagcagcagcagcagcagcaacaacaacaacaacaacagcaacaaatgcctcaaatgcaacagcaacaaaatcagcaacaaatgcaacagcaacaaaatcagcaacaaatgaatcaagcttatatgagtcaaggtggtggccagaggtctcaaattatgtctcaaggaggacaagcttcgtcgcaaggaccacctccgactatgcctggaggaccctttattaattaa

SEQ ID NO: 25 - protein, 777 aa, Mimulus guttatus
MVPKQLIVAVEGTAAIGPFWQTIVSDYLDKIIRSFCGNEPTGQKPATTHFELSLVMFNAHGSYSACLVQRSGWTKDMDIFFQWLSAIPFAGGGFNDAAIAEGLSEALMMFSSPKGNQNQNVEGQRHCILIAASNPYPLPTPVFRPQIQNPEKNDGVESQPDNRLSDAETLAKSFAQCAVSLSVICPKQLPKLRGIFNAGKRNPRAPDPPVDNNKNPHFLVLISENFMEARSCLSRSGITSLPPNQIPVKMDVTPAPPPISGPPPVSIPAVNGSLLARQPVPAANIPPAIVKLEPPTIAPSMTGPTFPHISSIPRAVPQTVPTMQTSSPISTPQEVIPINENIPEVKPLVASSAPPIRPAGGAAANVRILNDMAQARQALAAAAAGGGGGASIGIPSMGGGTTMLSNMISSGMMTSSSMSSGPSIVTSISGPLPMSANGQVAQNNSGNSGIAMSSQPLSGLQGNVGPGQTGNIPVVPQMVQTGMGMNANMMSGGGGASGMSQQGQPPGGMNSNNVVNTLNQQTSATMQSAQSKYVKVWEGNLSGQRQGQPVFITRLEGYRSASASETLAANWPSTMQIVRLISQDHMNNKQYVGKADFLVFRAMNQHGFLGQLQEKKLCAVIQLPSQTLLLSVSDKAHRLIGMLFPGDMVVFKPQIPNNNNNNNNNQQQQQQMQVQLQQQQMQVQLQQQQIQQQQQPIMQIQQQQQQQQQQQQQQQQQMPQMQQQQNQQQMQQQQNQQQMNQAYMSQGGGQRSQIMSQGGQASSQGPPPTMPGGPFIN

SEQ ID NO: 26 - DNA, 2529 bp, Oryza sativa
  CDS (1)..(2529): Oryza sativa cDNA encoding Med25 protein
atggcggcggcggcggccgagaggcagctggtggtggccgtggaggggacggcggcgctggggccgtactggcccgtcaccgtggcggactacgtcgagaagatcgtgcggagtttttgtgcacatgaaatggcaggacagaagctcgcagggacaccccctgaacttgcattagtcgtcttccatacccatggtccttatagcgctttttgtgtgcaacggagtggatggacaaaagatatgaatgtgtttctttcatggttatctggaatatcatttagtggtggaggctttagtgaagctgctatttctgaaggtcttgctgaagcattgatgatactccaaggcagttctagtaacagtcagaatcatcaaagccatgaagtacaaaaacattgcatacttgttgcagcaagtaatccttatccactgcctacgcctgtctaccgcccccttgttcaaagtagcgatcacaaggagaacaatgatggagcaaaagaatcttgtcttgctgatgctgagactgttgcaaaatcatttgctcagtgctccgtttcattgtcggtggtatctcctaaacagcttccaactctgaaagcaatatacaatgcggcaaagaggaatcctcgagcggctgacccatcagtggatcatgcaaaaaatccacattttcttgttttgttgtctgacaattttttggaggctcgaactgctctaagtcgccctttacctggcaacttggtcacaaatcaccccattacaaaaatggatacagctgcaacatctgtgccagtaccaacttcaaatggcaacccctcagttaatggacctatgcttacccgccaaccaaatggtgttgttgcaaatattaaaacggagccaacaactttaccgcccatggtttctgcacctgctttctcgcatgtaacacctgttgcaaatggtgtttcacaaggattatcatcagtacaaagtccctcaccgtcccttatttcacaggaaactaatcttgcaaatgatagtgtgcaagaacataagcctttaataaaccctatccaacagtcaattcgacctggtggtccagcaaatgtcagcatcctcaacaatctatcacagcatcggtcagtggcaaccattatatcaggtggaatgcctggcatccctatgtctggaacaggacagtcaattggtagtcaacaagtcgtacaaaacactgcttttggatcaaacacacccataacaggcaattcaaatattgctgtgtcatcttctttgggtggcatccaaagcaatatcggtatatcagggcctcctgtgacacagggaggttcaatgggtagtacgcaattgggacaaggtggaatcaatacaaaccaaaatatgataagtagccttgggacaacaactgtctcttctgcacctgcaatgatgccaacaccagggatggctcaacaggcaggtgtaaattctcttggtgtgaccaacagttctgccatgaacatgcctatagtgcagcatcctaatgcgcagcagcagcaacagcaacagcaacagcagcagcagcagcagccaccgccgaagtacgtcaaaatttgggagggaactttatctgggcaaaggcaaggacaacctgtatttatctgtaaacttgaaggttacaggagtggaacagcatctgaaacacttgcagcagactggcctgaaacaatgcagattgtgcgccttatagctcaggagcatatgaacaataaacaatatgttggaaaagcagactttctagtatttcggacattaaatcagcacggcttccttgggcaactgcaggaaaagaagctgtgcgcagtgattcaactgccttcgcaaactttgttgttgtcagtgtcagacaaagctgggcgcctcattggcatgctgttccctggggatatggtggtgtttaaaccgcaggtaccaacccagcagccaccaatgcagcaacaacagttacaacagcagcagaaccaactacaacagcagaatcagctccaccagcagcaccagctgcaaccacagaaccagctgcaacagcaacaccagctgcaacaacagttacaacagcagcaactacaacaacacatgcaactgcagacacaaggccttccgcttcagcagcagcaatcccaaggccatccgcttcagcagcagcagatgcagcaaatgcagcaacaacagcagcagcagcagattcagcaaatgcagcagcagcagcagatgcagcagatgcaacagcagcagcagcagccccaacagcttcagcagcagcagcaaccgcagatggtcggcacagggatggggcagcagcaaccacagatggtcggcacggggatggggcagcagcaaccgcagatggtcggcgcagggatggggcagcaatacatgcaggggcacggtaggacggtgcagcagatgatgcaagggaagatggcgccgcagggtccaggaagcatgccgggtgcagggagcatgcctgggggtggctacctatcttga

SEQ ID NO: 27 - protein, 842 aa, Oryza sativa
MAAAAAERQLVVAVEGTAALGPYWPVTVADYVEKIVRSFCAHEMAGQKLAGTPPELALVVFHTHGPYSAFCVQRSGWTKDMNVFLSWLSGISFSGGGFSEAAISEGLAEALMILQGSSSNSQNHQSHEVQKHCILVAASNPYPLPTPVYRPLVQSSDHKENNDGAKESCLADAETVAKSFAQCSVSLSVVSPKQLPTLKAIYNAAKRNPRAADPSVDHAKNPHFLVLLSDNFLEARTALSRPLPGNLVTNHPITKMDTAATSVPVPTSNGNPSVNGPMLTRQPNGVVANIKTEPTTLPPMVSAPAFSHVTPVANGVSQGLSSVQSPSPSLISQETNLANDSVQEHKPLINPIQQSIRPGGPANVSILNNLSQHRSVATIISGGMPGIPMSGTGQSIGSQQVVQNTAFGSNTPITGNSNIAVSSSLGGIQSNIGISGPPVTQGGSMGSTQLGQGGINTNQNMISSLGTTTVSSAPAMMPTPGMAQQAGVNSLGVTNSSAMNMPIVQHPNAQQQQQQQQQQQQQQPPPKYVKIWEGTLSGQRQGQPVFICKLEGYRSGTASETLAADWPETMQIVRLIAQEHMNNKQYVGKADFLVFRTLNQHGFLGQLQEKKLCAVIQLPSQTLLLSVSDKAGRLIGMLFPGDMVVFKPQVPTQQPPMQQQQLQQQQNQLQQQNQLHQQHQLQPQNQLQQQHQLQQQLQQQQLQQHMQLQTQGLPLQQQQSQGHPLQQQQMQQMQQQQQQQQIQQMQQQQQMQQMQQQQQQPQQLQQQQQPQMVGTGMGQQQPQMVGTGMGQQQPQMVGAGMGQQYMQGHGRTVQQMMQGKMAPQGPGSMPGAGSMPGGGYLS

SEQ ID NO: 28 - DNA, 2391 bp, Populus trichocarpa
  CDS (1)..(2391): Populus trichocarpa cDNA encoding Med25 protein
atggcggagaaacagttgatcgtggccgttgaaggcactgctgccatgggccctttctggtcgatcatcgtttctgattaccttgaaaagatcatcaggtacaaggtccctacttccattgttgagctctcaattgtcacgtttaattctcatggatcatattctgcttgcctagtacaacggagtggctggacaagagatgttgatatcttcttacagtggctatcagcaattccctttgctggtggtggtttcaatgatgctgcaattgcagaagggctttctgaagctctaatgatgttccctattgatccaaatggaagccaaactcaatcaaatatagacgagcaaaggaactgtattcttattgctgcaagtaacccacacccattgcccacaccagtatatcgtccacaaatacaaaatttggagcagattgaaaatattgatgcacagaatgaaagtcgtctatctgatgcagagacagttgctaaatcatttcctcagtgctcggtttctctttcaatcatttgtccaaaacagcttccaaaacttaggtcaatctacaatgctgggaagcgcaacagccgagcagcagatccacctgttgacagtgtaaaaaatcctcattttcttgtgttaatctcagagaactttatggaggctcgtgctgcattgagtcttcctggagttacaagtttgacttctaatcaaactcctgtaaaagtggatattgcttcagttacttctgtaactgggccagctccaacttctattccgtcagtgaatggatctatcacaaatcgaccaacaatttcagttggaaatgttcccactgcaactgtgaaagttgagccaagtactataacttccatggcaaatggacctactttcccccataatccttctgttccacgccctgcatctcaaggagttccaatcttgcagacttcttcaccatctactactactcaagatatggcaacaagtggtgatgatgtgcaggacttgaaaccaaatgtcagtgtgatgactcagtctgcacgtcctgggcctcctgctgcagcaaatgtgagcattctgaacaatatctctcaagcacggcaagtgatgaactctgcaggcttaagtggaggaacttctttaggtctcccatcaataaatcaaacttctgtggccatgcatatgtcaaatatgatttcgagtggaatggcatcttctgtgcctgctgccccaactgtattttcatctgggcaaccaggtgtctcttcgataacagggtcagggactcttactgggacaacgcaaattgggccaaactctggtcttggatcattcacttctgctacttctaatgtgtctggaaattcaaaccttgggaggatttcacaaccaatgggtaatcttcaagcaggtgttagcattggccaatcagcaccaggcatgagccaaggaaatatttcaggagcacaaatggtacaaagtgggatgggtgcaaacccaaacacaatgagcggtctgggtccatcaggtgtctcttctggtttgaacacgatgattcctacccctggaatgtctcaacaagtacaatccgggatgcagcctcttggtgcaaacaataactcagcagataatttgtcaatgtcacaacaaacagcagggggcttgcaaccaccacaatcaaaatatgtcaaagtctgggagggaaacttgtctgggcaacggcaagggcagcctgtctttatcaccagattagaaggttataggagtgcttcggcctcggagacgcttgcagcaaactggccaccgacaatgcaaatagttcggcttatatctcaggaccacatgaataacaagcaatatgtcggaaaggctgattttctagtttttcgggccatgaatcagcatggatttcttggacaactccaagagaagaagctttgtgcagtaatccagttaccatctcagacattgcttctctctgtttctgacaaagcctgccgcttgatagggatgctttttcctggggatatggttgtgtttaagccacaaatatccagtcagcagcagcagatgcaaatgcaacggcagcagcagcagcagcagatgcaacaacaacaacaacagcaacaacatccacaactgcagcagcagcaacatcagcagcagcttccacaattgcaacagcagcagctgtcgctgctgcaacaacagcagctgtcgctgctgcaacagcagcagcagcttccgcaattgcagcaacagcaaatggctggaacaggaatgggtcaaacttatgttcaaggtccaggtcgtccacaattagtttctcaaggacaagtttcttcacaagggccagcaaacatgcctggaggaggctttatgagttaa

SEQ ID NO: 29 - protein, 796 aa, Populus trichocarpa
MAEKQLIVAVEGTAAMGPFWSIIVSDYLEKIIRYKVPTSIVELSIVTFNSHGSYSACLVQRSGWTRDVDIFLQWLSAIPFAGGGFNDAAIAEGLSEALMMFPIDPNGSQTQSNIDEQRNCILIAASNPHPLPTPVYRPQIQNLEQIENIDAQNESRLSDAETVAKSFPQCSVSLSIICPKQLPKLRSIYNAGKRNSRAADPPVDSVKNPHFLVLISENFMEARAALSLPGVTSLTSNQTPVKVDIASVTSVTGPAPTSIPSVNGSITNRPTISVGNVPTATVKVEPSTITSMANGPTFPHNPSVPRPASQGVPILQTSSPSTTTQDMATSGDDVQDLKPNVSVMTQSARPGPPAAANVSILNNISQARQVMNSAGLSGGTSLGLPSINQTSVAMHMSNMISSGMASSVPAAPTVFSSGQPGVSSITGSGTLTGTTQIGPNSGLGSFTSATSNVSGNSNLGRISQPMGNLQAGVSIGQSAPGMSQGNISGAQMVQSGMGANPNTMSGLGPSGVSSGLNTMIPTPGMSQQVQSGMQPLGANNNSADNLSMSQQTAGGLQPPQSKYVKVWEGNLSGQRQGQPVFITRLEGYRSASASETLAANWPPTMQIVRLISQDHMNNKQYVGKADFLVFRAMNQHGFLGQLQEKKLCAVIQLPSQTLLLSVSDKACRLIGMLFPGDMVVFKPQISSQQQQMQMQRQQQQQQMQQQQQQQQHPQLQQQQHQQQLPQLQQQQLSLLQQQQLSLLQQQQQLPQLQQQQMAGTGMGQTYVQGPGRPQLVSQGQVSSQGPANMPGGGFMS

SEQ ID NO: 30 - DNA, 2691 bp, Unknown
  Populus2
atggcggacaaacagctgatcgtggccgttgaaggcacggccgccatgggccctttctggtcgatcatcgtttctgattatcttgaaaagattatcaggtgttattgtggtaatgaaacgtcagggcagaagccccctacttctagtgctgagctctctcttgtcacgtttaattctcatggatcatattctgcttgcctggtacaacgtagtggctggacaagagatgttgacatcttcttacactggctatcagccattccttttgctggtggcggtttcaatgatgctgcaattgcagaagggctttctgaagctctaatgatgttccctattactccaaatggaaaccaaactcaaccaaatacagatgggcaaagaaattgtattcttattgctgcaagtaaccctcacccattgcccactccagtctatcgtccacaaatacaaaatttggagcagactgaaaacatcgatgcacagaatgaaagtcgtctatctgatgcagagacagttgctaaatcatttcctcagtgctcggtttctctttcagtcatctgtccaaaacagcttccaaaacttagattaatctacaatgcagggaagcgcaacagccgagcagcagatccacctgtcgacaacataaaaaatcctcattttcttgtgctgatctcagagaactttatggaggctcgtgctgcattaagtcgtcctggagctacaagtttgccttctaatcaaacacctgttaaagtggatattgcttcagttacttcagtaactgggccacttccagcttcaattccttcagtgaatggatctatcacaaatcggccgccaatttcagttggaaatcttccaactgcaactgtgaaagtcgaaccaagtactataacttctatggcaaatggatctgcttttccccatattacttctgttccacgcccttcatctcaaggagttccaagcatgcagacttcatcaccatccacgactactcaagatatggcaacaaacggtgaaaatgcgcaggacttgaaacctaatgtcagtgtgacgacacagtctgcacgttctgcgcctcctgctgcagctaacgtgaacattctgaacaatctttctcaagcaaggcaagtgatgaactctgcagccctaagtggaggaacttctattggtctcccatcaataaatcaaacttctgtggctatgcatatgtcaagtatgatttcaagtggaatgacgtcttccgtgcctgctgctcaaactgtattttcatctgggcaaccaggtgtttcttcaataacagggtcagggaatcttactgggacaacacaaatcacaccaaactctggtctgggctcattcacttccgcaacttctaacatgcctggaaactcaaaccttggggggatttcacaaccaatgggtaatcttcacgcaggtgttagcatcagccaatcagcaatgagtggtctgggtacaaacccaaacacaatgagcggttcaggagcacaaatggtacaaagtggaatgggtgcaaaccccaacacaatgagtggtctgggtgcttcaggtgtctcttctggttcgaacacaatgattcctactccaggaatgtctcaacaagtacaatctgggatgcagcctcttggtgcaaacaataactcagcagctaatttgccattgtcacaacagacagcagggggcttgcaaccagctcaatcgaaatatgtcaaagtttgggagggaaacttgtctgggcaacggcaagggcagcctgtctttatcactagactagaaggttataggagtgcttcggcctctgagacgcttgcagcaaactggccgccaacaatgcaaatagttcggcttatatctcaggatcacatgaataacaagcaatatgttggaaaggctgattttctagtatttcgggctatgaatcagcatggatttcttggacaactccaagagaagaagctttgtgcagtaatccagttaccatctcagacattgctactctctgtttctgacaaagcctgccgcttgatagggatgctttttcctggggatatggttgtgtttaagccacaaatatccagtcagcagcaacaaatgcaacagcagcatcaccaacagatgcaacaacaacaacatccacaactacagcagcttcaacatcagcagcagctcccacaactacagcagcagcaacagctctcacagctacagcagcagcagcagcagctctcacagcttcaacagcagcagccacagcagctctcacagcttcaacagcagcagccacagcagctacctcaattgcagcagcagcagcagctctcacagcttcaacagcagcagcttccacaaatgcagcagcagcttccacaaatgcagcagcagcagcaattgcagcagcagcagcaaatgcagcagcagcagcaaatgcagcagcagcagcaattgcagcagcagcagcagcagcaattgcagcagcagcagcagcagcaaatgcagcagcagcagcagcagcagcttcagcaaatgcagcagcagcagcaaatggttggaacaggaatgggtcaaacttatgttcaaggtccaggtcgttcacaattagtttctcaaggacaagtttcttcacaagggccagcaaacatgtctggaggaggctttatgagtcaataa

SEQ ID NO: 31 - protein, 896 aa, Unknown
  Populus2
MADKQLIVAVEGTAAMGPFWSIIVSDYLEKIIRCYCGNETSGQKPPTSSAELSLVTFNSHGSYSACLVQRSGWTRDVDIFLHWLSAIPFAGGGFNDAAIAEGLSEALMMFPITPNGNQTQPNTDGQRNCILIAASNPHPLPTPVYRPQIQNLEQTENIDAQNESRLSDAETVAKSFPQCSVSLSVICPKQLPKLRLIYNAGKRNSRAADPPVDNIKNPHFLVLISENFMEARAALSRPGATSLPSNQTPVKVDIASVTSVTGPLPASIPSVNGSITNRPPISVGNLPTATVKVEPSTITSMANGSAFPHITSVPRPSSQGVPSMQTSSPSTTTQDMATNGENAQDLKPNVSVTTQSARSAPPAAANVNILNNLSQARQVMNSAALSGGTSIGLPSINQTSVAMHMSSMISSGMTSSVPAAQTVFSSGQPGVSSITGSGNLTGTTQITPNSGLGSFTSATSNMPGNSNLGGISQPMGNLHAGVSISQSAMSGLGTNPNTMSGSGAQMVQSGMGANPNTMSGLGASGVSSGSNTMIPTPGMSQQVQSGMQPLGANNNSAANLPLSQQTAGGLQPAQSKYVKVWEGNLSGQRQGQPVFITRLEGYRSASASETLAANWPPTMQIVRLISQDHMNNKQYVGKADFLVFRAMNQHGFLGQLQEKKLCAVIQLPSQTLLLSVSDKACRLIGMLFPGDMVVFKPQISSQQQQMQQQHHQQMQQQQHPQLQQLQHQQQLPQLQQQQQLSQLQQQQQQLSQLQQQQPQQLSQLQQQQPQQLPQLQQQQQLSQLQQQQLPQMQQQLPQMQQQQQLQQQQQMQQQQQMQQQQQLQQQQQQQLQQQQQQQMQQQQQQQLQQMQQQQQMVGTGMGQTYVQGPGRSQLVSQGQVSSQGPANMSGGGFMSQ

SEQ ID NO: 32 - DNA, 2577 bp, Sorghum bicolor
  CDS (1)..(2577): Sorghum bicolor cDNA encoding Med25 protein
atggcggctgcagataggcagcttgtggttgccgtcgagggaacggcggcgctggggccctactggtccaccattgtggcggagtacgtcgagaagatcgtgcggagcttttgtgcaagtgaactgccaggccagaagcttgcagggcctccgcctgagcttgcattagttgtttttcatacccatggaccctacagtgcttttgatgtgcagcgaagtggatggacaaaagataccgatgctttcctatcatggttatctggaatatcattcagtggtggaggattcagtgaagcttctacttgtgaaggtcttgcagaagcactgaagatacttcaaggtagtcctaatgcaacccaaagtcatcaaaaccatgaagcacaaaagcattgcatacttgtcgctgcaagtaatccttatcctttgccgacacctgtatactgtcttcctactcaaagtactgatcacaaagagaacattgagacatcaaaagaaccttctattgctgatgctgagaccgttgcaaagtcatttgctcagtgctctgtctcactgtcggtgatatctccaaaacagcttccaacattaaaggcgatatacaatgcgggcaagcgaaaccctcgagctgctgatccatcagtcgaccatgcgaaaaatccacacttccttgttttgctatctgagaatttcatggaggcgcgaactgctctaagtcgtcctttacatggaaacatggccccaaatcaaagcattacaaagatggacactgcacctgcagtgactatgccaggaccgacttcaaatgccaacccctcaggccgtcaaccagttgttggaggtatttctaccgcaactgttaaactggaaccagcaaccatgccaccaatagtttctgcacctgctttctcacatgtaacacctatttcaaatgtggcttcacaaggaatatcagcacttcaaacctcatcaccatctcttatttcacaagaagcaaatatggcaaatgataatgtgcaagaacacaagcctataataaaccctgttcaacagccagttcggcctggtggtcatggcagcctcctcaacaatctgtcacaggttcggttgatgaactcaacttctttaggaggaggagctacctcaatgggacttcctaacatgggggcaacaccaatacaagtccacatgtcaaacatgatatcaagcggcatgacatcaacaccctctgtcatctcctctatgtctggacctggacaacctattggtactcaacagatggtacagagcacagctcttggttcctttggctcaaacacttctactgtatcaggcaattcaaatgttgctgtatcatcctctctgactaacaaccaaagcagtatgggcatgggtcaatcagtgcaacctgtggcacaaggaggcttggtggctggttcacaattaggacaaggtggaattggtgcaaaccaaaatgtgatgagtagccttggatctacagctatctcttcagcacctgcgatgatgccaacacctgggatggtcccacagacaggagtcaattccttaggtgtgaataacaatcctgctatgaatatgcctattccacagcatgccaatgcccaacagccgccaccaaagtatgtcaaaatttgggagggaactttatctggccaaaggcaaggacagcctgtatttatttgtaaactcgaaggttataggagtggaacagcatctgaaacacttgcagcggactggccagaaactatgcagattgttcgcctcatagcccaggagcatatgaacaacaaacaatatgttggcaaggccgactttcttgtttttcggactttaaatcagcacgggtttcttggacaactacaagagaagaagctgtgtgctgtgatacagttaccatcgcaaactctgctgctgtcaatgtctgacaaagcaggtcgtttgattggcatgctcttccctggggacatggtggtgttcaaaccgcaggtcccaactcagcagacaccaatgcagcagcagcagctacaacaacagcaacttcaacaacaacagttacagcagcagcagctgcaacaacaacacatgcatatgcagcagcaaggcctcccacttcagcagtcgcagatgcaacttcagcaacagcagcagccacagatgcaaccgatgcagcagcagccaacacagatgcaacaccagcagcagcaaccttcgcagatgcagccgatgcagcatcagcagccgcagccacagcagatgcaatcgatgcagcaccagcagcagatgcagcaccagcagcagcagcagcagcagatgcaaccaatgcagcaacagcagcagatgcaacacatgcagcatcaacagcaacagatgcagcaaatgcaacagcagcagcaacagatgcagcaaatgcagcctcaacagcagcagcaaccccagatggtgggtacaggaatggggcagcagttcatgcaggggcagaatcgggcagtgcagatgatgcaagggaagattatgccacagggcccgggcagcatgcctgggggaggcttcctaccttaa

SEQ ID NO: 33 - protein, 858 aa, Sorghum bicolor
MAAADRQLVVAVEGTAALGPYWSTIVAEYVEKIVRSFCASELPGQKLAGPPPELALVVFHTHGPYSAFDVQRSGWTKDTDAFLSWLSGISFSGGGFSEASTCEGLAEALKILQGSPNATQSHQNHEAQKHCILVAASNPYPLPTPVYCLPTQSTDHKENIETSKEPSIADAETVAKSFAQCSVSLSVISPKQLPTLKAIYNAGKRNPRAADPSVDHAKNPHFLVLLSENFMEARTALSRPLHGNMAPNQSITKMDTAPAVTMPGPTSNANPSGRQPVVGGISTATVKLEPATMPPIVSAPAFSHVTPISNVASQGISALQTSSPSLISQEANMANDNVQEHKPIINPVQQPVRPGGHGSLLNNLSQVRLMNSTSLGGGATSMGLPNMGATPIQVHMSNMISSGMTSTPSVISSMSGPGQPIGTQQMVQSTALGSFGSNTSTVSGNSNVAVSSSLTNNQSSMGMGQSVQPVAQGGLVAGSQLGQGGIGANQNVMSSLGSTAISSAPAMMPTPGMVPQTGVNSLGVNNNPAMNMPIPQHANAQQPPPKYVKIWEGTLSGQRQGQPVFICKLEGYRSGTASETLAADWPETMQIVRLIAQEHMNNKQYVGKADFLVFRTLNQHGFLGQLQEKKLCAVIQLPSQTLLLSMSDKAGRLIGMLFPGDMVVFKPQVPTQQTPMQQQQLQQQQLQQQQLQQQQLQQQHMHMQQQGLPLQQSQMQLQQQQQPQMQPMQQQPTQMQHQQQQPSQMQPMQHQQPQPQQMQSMQHQQQMQHQQQQQQQMQPMQQQQQMQHMQHQQQQMQQMQQQQQQMQQMQPQQQQQPQMVGTGMGQQFMQGQNRAVQMMQGKIMPQGPGSMPGGGFLP

SEQ ID NO: 34 - DNA, 2280 bp, Triticum aestivum
  CDS (1)..(2280): Triticum aestivum cDNA encoding Med25 protein
atggtggaggggatggcttcggagaggcagctggtggtggtcgttgagggcacggcggcgctggggccgtactggcccgccattgcggccgagtacgtcgagaagatcgttcggagtttttgttctactgaactatcagggcagaagcttgcaggggtaccacctgagcttgcattagttgtcttccatacccacggaccatacagtgcctttattgtacaacgcagcggttggacaaaagatatggatgcttttctttcatggttatcaggaatatcatttagtggtggaggcttcagtgaagctgctatttgtgaaggtcttgctgaagcactgatgatactccaaggcagttctagtagcagccagaatcatcaaaatcgtgaacttcaaaagcattgcctacttgttgctgcaagtaatccttacccgctgcctacacctgtctaccgcccttctgttcaaagtagtgatcacaaaaagagcaacgaagcaacaaaggaatcatgtcttgctgatgctgaggctgttgcagtctcatttgctcagtgctctgtgtcattgtcggtggtatctcctaaacagctaccaacactgaaggcaatatacaacgcgggaaagaggaatcctcaagcttctgatccatcagttgatcatgccaaaaatccacattttcttgttttgctctctgagagtttcatggaggctcgaacagctctaagccatcctttacccgggaaccttgtcccaaaccacaccattacaaaaatggataccacacctgcagctactgtgccaggaccaccttcgaatgccagtccctcagtgaatggaacgatgatgggacggcaaccgactgcaaatgttaaagtggagccaacaactataccaccaatggtttctgcacctgcattctctcatatgacacctatttcaaatgttgcttctcaaggtgtatcagcaatgcaaacttcttcaccatcgattatttcacaagaaacaaatgttgcaaatgaaatattgcaagagcataagcctttagtaaaccctatccaacaacaggttcggcctggtggcccagcaaatgttagcatcctaaacaatctgtcacaacatcgacactctttaacagcagctacctcaatgggacctaacatgggagcaacacctatacaagtgcacatgtcaaacatgatatcgagtggcatgacatcaactccggctgtcatatcttctatatctggaacagtacagccaattggtgctcaacagttggcacagaatacagctctcggttcttttgggtcaaacacttctactgtatcttgtaattcaaatatcgccgtatcgtcttctttagctaacatacaaagcaacatggccatgggacaatcagtgccttctatggcacaaggtggtctgatggctggtccacaatcaggacaaggtggaattggtacgaaccaaaacatgataaataaccttgggactacagctatcaattctacgcctacaatgatgccaacaccaggaatggtccagcaaacaggagtaaatgcccttagtgcgaacaacagttctgctatgaatatgcctctggcacaacatcctaatggccagcaaccatcgaagtatgtcaaaatttgggagggaactttgtctgggcaaaggcaaggacagcctgtatttatttgtaaacttgaaggttataggagtggaacagcgtctggcacacttgcttcagactggccagaaacgatgcagatcgtgcgcctcatagcccaagagcatatgaacaacaagcagtatgttggcaaggcagactttctagtatttcggacattgaatcagcatgggttcctcggacaactacaagagaagaaactgtgtgcagtgattcagctaccttcacaaactttgctgttgtcagtgtctgacaaagctgggcgcctcataggaatgctgttcccaggggatatggtggtattcaaaccacaggtctcaactcagcagccacagatgcagccacagcagcagcagcagcagcagcagctacagcagcagcaccaccaaatacagcagcaacagcaacaacagatgcagcaaatgcagcagcagcagcagcagcctcaacaactccagcagcagccgcagatggtgggtacagggatgggtcagcaacaattcatgcaggggcatggtcgggcggtgcagatgatgcaaggaaagatcgcgccacagggcccaggcaacatgtctggaggaggctacctatcttaa

SEQ ID NO: 35 - protein, 759 aa, Triticum aestivum
MVEGMASERQLVVVVEGTAALGPYWPAIAAEYVEKIVRSFCSTELSGQKLAGVPPELALVVFHTHGPYSAFIVQRSGWTKDMDAFLSWLSGISFSGGGFSEAAICEGLAEALMILQGSSSSSQNHQNRELQKHCLLVAASNPYPLPTPVYRPSVQSSDHKKSNEATKESCLADAEAVAVSFAQCSVSLSVVSPKQLPTLKAIYNAGKRNPQASDPSVDHAKNPHFLVLLSESFMEARTALSHPLPGNLVPNHTITKMDTTPAATVPGPPSNASPSVNGTMMGRQPTANVKVEPTTIPPMVSAPAFSHMTPISNVASQGVSAMQTSSPSIISQETNVANEILQEHKPLVNPIQQQVRPGGPANVSILNNLSQHRHSLTAATSMGPNMGATPIQVHMSNMISSGMTSTPAVISSISGTVQPIGAQQLAQNTALGSFGSNTSTVSCNSNIAVSSSLANIQSNMAMGQSVPSMAQGGLMAGPQSGQGGIGTNQNMINNLGTTAINSTPTMMPTPGMVQQTGVNALSANNSSAMNMPLAQHPNGQQPSKYVKIWEGTLSGQRQGQPVFICKLEGYRSGTASGTLASDWPETMQIVRLIAQEHMNNKQYVGKADFLVFRTLNQHGFLGQLQEKKLCAVIQLPSQTLLLSVSDKAGRLIGMLFPGDMVVFKPQVSTQQPQMQPQQQQQQQQLQQQHHQIQQQQQQQMQQMQQQQQQPQQLQQQPQMVGTGMGQQQFMQGHGRAVQMMQGKIAPQGPGNMSGGGYLS

SEQ ID NO: 36 - DNA, 2748 bp, Zea mays
  CDS (1)..(2748): Zea mays cDNA encoding Med25 protein
atggcggcggcggataggcagttggtagttgccgtcgaggggaccgcggcgctggggccctactggtccaccattgtggcggagtacgtcgagaagatcgtgaggagcttttgtgcaagtgaactgccaggccagaagcttgtgggtactccgcctgaacttgcattagttgtttttcatacccatggaccttacagtgcttttgatgtgcagcgaagtggatggacaaaagatgtcgatgctttcctatcatggttatctggaatattgttcagtggtggaggattcagtgaagcttctacttgtgaaggtcttgctgaagcattgaagatacttcaaggcaatcctaatacaacccaaggtcatcaaaaccatgaagcacaaaagcattgcatacttgtcgctgcaagtaatccttatcctctgccaacacctgtatactgtcttcctactcaaagtactgatcacaaagagaacattgagacatcaaaagaaccttcaattgctgatgctgagaccgttgcaaagtcatttgctcagtgctctgtctcattgtcagtgatatctccaaaacagcttccaacattaaaggcaatatacaatgcgggcaagcgaaatcttcgggctgctgatccatcagtcgaccatgcgaaaaatccacacttccttgttttgctctctgagaatttcatggaggcacggactgctcttagtcgccctttacatggaaacttggctccaaatcaaaccataacgaagatggacacggcacctgcagtgactatgccaggaccaacatcaaatggcaactcctcaggccgtcaaccaattgttggaggtatttctaccacatctgttaaagtggagccaacaaccatgccaccgatagtttctgcacctgctttctcgcatgtaacgcctatttcaaatgtggcttcacaaggaatatcagcactgcaaacctcatcaccttctcttatttcacaagaagcaaatatgggaaatgataatgtacaagaacacaagcctataataaacccagttcaacaaccgattcggcctggtggtcatggcagcctcctcaacaatctgtcacaggttcggttgatgaactcaacttctttaggaggaggagctacctcgatgggactgcctaacatgggagcaacaccaatacaagtccacatgtcaaacatgatatcaagcggcatgacatcaacaccctctgtcatctcctctatgtctggacctggacaacctattagtactcaacagatggtacagagcacagctcttggttcgtttggctcaaacactcctactgttacaggcaattcaactattgctgtatcatcctctttgactaacaaccaaagcagcatgggcatgggtcaatctgtgcaatctgtggcacagggaggcttggtgtctggttcacaattaggacaaggtggaattgttgcaaaccaaaatgtgatgagcacccttggacctacagctatctcttcaacgcctgcgatgatgccgacacctgggatggtcccacagacaggagtcaattccttaggtgtgaataacaattctgctatgaatatgcctattacgcagcagcatgccaatgcccaacagccgccaccaaagtacgtcaaaatttgggagggaactttatctggccaaaggcaaggacagcctgtaataatttgtaaactcgaaggttataggagtggaacagcgtctgaaacacttgcagcggactggccagaaacaatgcagattgttcgcctcatagcccaggagcatatgaacaacaaacaatatgttggcaaggcagactttctagtttttcggactttaaatcaccatgggtttcttgtacaactacaagagaagaagctgagtaattttgttattttgctgcggttcatctactgtcgtttcatacacttagttttctgtcagttactttatacagttctaatatccaattggtcctggtttcagtgtgctgtgatacagttaccatcgcaaactctgttgctgtcgatggctgacaaagcaggtcgtttgattggcatgctcttccctggggacatggtggtgttcaaaccgcaggcctcaactcagcagacaccaatgcagcaacagcagctacagcaatttcaacagcagcagcagcagctgcaacaacacatgcatatgcagccgcaaggcctcccacttcagcaatcacagatgcaactccagcaacagcagccgcagatgcaaccgatgcagcagcagcaaccttcgcagatgcagcagcagatgcaatcgatgcagcagcagatgcagccaatgcagcagcaaatgcagcatcaacagcaacagatgcagcatcaacagcaacagatgcagcaaatgcagcagcagcagcagcaacaacaacaacaacaacaacagatacagcctcagcagcaacagatgcagcaaatgcagcagcagcagcagcaacagatgcagcctcaacagcaacagatgcagcaaatgcagcagcagcagcaacagatgcagcctcaacagcaacagatgcagcaaatgcagcagcagcagcaacagatgcagcctcaacagcagcagcaaccccagatggtgggtacaggaatggggcagcagttcatgcaggggcacaatcgggcagtgcagatgatgcaagggaagattacgccacaggggccgggcagcatgcctgggggaggcttcctaccttaa

SEQ ID NO: 37 - protein, 915 aa, Zea mays
MAAADRQLVVAVEGTAALGPYWSTIVAEYVEKIVRSFCASELPGQKLVGTPPELALVVFHTHGPYSAFDVQRSGWTKDVDAFLSWLSGILFSGGGFSEASTCEGLAEALKILQGNPNTTQGHQNHEAQKHCILVAASNPYPLPTPVYCLPTQSTDHKENIETSKEPSIADAETVAKSFAQCSVSLSVISPKQLPTLKAIYNAGKRNLRAADPSVDHAKNPHFLVLLSENFMEARTALSRPLHGNLAPNQTITKMDTAPAVTMPGPTSNGNSSGRQPIVGGISTTSVKVEPTTMPPIVSAPAFSHVTPISNVASQGISALQTSSPSLISQEANMGNDNVQEHKPIINPVQQPIRPGGHGSLLNNLSQVRLMNSTSLGGGATSMGLPNMGATPIQVHMSNMISSGMTSTPSVISSMSGPGQPISTQQMVQSTALGSFGSNTPTVTGNSTIAVSSSLTNNQSSMGMGQSVQSVAQGGLVSGSQLGQGGIVANQNVMSTLGPTAISSTPAMMPTPGMVPQTGVNSLGVNNNSAMNMPITQQHANAQQPPPKYVKIWEGTLSGQRQGQPVIICKLEGYRSGTASETLAADWPETMQIVRLIAQEHMNNKQYVGKADFLVFRTLNHHGFLVQLQEKKLSNFVILLRFIYCRFIHLVFCQLLYTVLISNWSWFQCAVIQLPSQTLLLSMADKAGRLIGMLFPGDMVVFKPQASTQQTPMQQQQLQQFQQQQQQLQQHMHMQPQGLPLQQSQMQLQQQQPQMQPMQQQQPSQMQQQMQSMQQQMQPMQQQMQHQQQQMQHQQQQMQQMQQQQQQQQQQQQQIQPQQQQMQQMQQQQQQQMQPQQQQMQQMQQQQQQMQPQQQQMQQMQQQQQQMQPQQQQQPQMVGTGMGQQFMQGHNRAVQMMQGKITPQGPGSMPGGGFLP

SEQ ID NO: 38 - DNA, 660 bp, Arabidopsis lyrata
  CDS (1)..(660): Arabidopsis lyrata cDNA encoding Med18 protein
atgtcaatggagtgtgtggttcaaggaatcattgagacacagcatgttgaagcgcttgagattcttcttcaaggtctttgtggtgttaaacgcgaacgtttaagagttcatgagttatgtttaaaaagtggcccaaatcttggtgttgtgtcttcagaagttaggctgttatgtgatcttgatcaaccagaacccacatggacggttaaacatgttggtggtccgatgagaggtgcaggagctgatcaaatctctgttatggttagaactatgattgaaagtaaagtaagcaagaatgctttgcgtatgttctatgcacttggttataaactagaccatgagcttctaaaagtcggattcgcctttcatttccaacggactgcgcacattagtgtttctgtatcttcggttaataagatgcctaaagttcatgctatagatgaggcagttcctgtaacacctgggatgcagattgttgatgttacagctcctgcaacacctgagaattacagtgaagtcgctgctgcggtttcatctttctgtgaatttctcgccccgcttgttcacttgtcgaaaccgtttatttccactggggttgtgccaaccgctgctgcggctgccgcatctcttatgtcggatggaggaggtactacattgtaa

SEQ ID NO: 39 - protein, 219 aa, Arabidopsis lyrata
MSMECVVQGIIETQHVEALEILLQGLCGVKRERLRVHELCLKSGPNLGVVSSEVRLLCDLDQPEPTWTVKHVGGPMRGAGADQISVMVRTMIESKVSKNALRMFYALGYKLDHELLKVGFAFHFQRTAHISVSVSSVNKMPKVHAIDEAVPVTPGMQIVDVTAPATPENYSEVAAAVSSFCEFLAPLVHLSKPFISTGVVPTAAAAAASLMSDGGGTTL

SEQ ID NO: 40 - DNA, 660 bp, Arabidopsis thaliana
  CDS (1)..(660): Arabidopsis thaliana cDNA encoding Med18 protein
atgtcaatggagtgtgtggttcaaggaatcatcgagacacagcatgttgaagctcttgagattcttcttcaaggtctttgtggtgttcaacgcgaacgtttaagagttcatgagttatgcttaagaagcggcccaaatcttggagttgtgtcatcagaagttaggctattatgtgatcttgatcaacccgaacccacatggacggttaaacatgttggtggtgcgatgagaggtgcaggagctgatcaaatctctgttttggttagaaatatgattgaaagtaaagtaagcaagaatgctttgcgtatgttctatgcactcggttacaaactagaccatgagcttctcaaagtcggattcgcctttcatttccaacggactgcgcacataagtgtttctgtatcttctgttaataagatgcctaaagttcatgctatagatgaggctgttcctgtaacacctggtatgcagattgttgatgtaactgctcctgcaacttctgagaattacagtgaagtcgctgctgctgtttcatctttctgtgaatttcttgctccgcttgttcacttgtcgaaaccgtctatctccacgggggttgtgccaaccgctgctgcggcggctgcatctcttatgtcggatggaggaggtactacattgtaa

SEQ ID NO: 41 - protein, 219 aa, Arabidopsis thaliana
MSMECVVQGIIETQHVEALEILLQGLCGVQRERLRVHELCLRSGPNLGVVSSEVRLLCDLDQPEPTWTVKHVGGAMRGAGADQISVLVRNMIESKVSKNALRMFYALGYKLDHELLKVGFAFHFQRTAHISVSVSSVNKMPKVHAIDEAVPVTPGMQIVDVTAPATSENYSEVAAAVSSFCEFLAPLVHLSKPSISTGVVPTAAAAAASLMSDGGGTTL

SEQ ID NO: 42 - DNA, 654 bp, Brachypodium distachyon
  CDS (1)..(654): Brachypodium distachyon cDNA encoding Med18 protein
atggagtgtgtggtgcagggtatcatcgagacccagcatgttgaagctcttgaggtccttctgcaggggctgtctggtgtcccaaaagagcgtgttagggttcacgagctctgtcttaaaagtgggcccaatctaggagttgtcccgtcagaggttcgtttgttgtgtgacttggctcagccaacaccatcctggaccataagacatgttggtggtgccatgagaggtgctggtgcagagcagatctcagttcttgtccgtacaatcgtagaaagcaaagccagcaacaatgtattgcgctacttctatggcataggttacaagttagatcatgaagttttgaaggtgggtttcgcatttcgtttccaacgaggtgcccagttcaccgtgactgtcacttccgccaacaagatgccgaaactccacgcgaccgacgaagctgtggcggtcactcctggaatacagcttgtggaaatcacagctcctgctgctgctaacaattacaacgacgtagcttcatctgtcactgcgttctgtgaatatctagcaccgctgctgcacctctctaaaccaggcaattcaactggaatcgtcctgactgctggtgctgctgctgcctcgctcatgtcaagtggtggtgctaaaacattgtaa

SEQ ID NO: 43 - protein, 217 aa, Brachypodium distachyon
MECVVQGIIETQHVEALEVLLQGLSGVPKERVRVHELCLKSGPNLGVVPSEVRLLCDLAQPTPSWTIRHVGGAMRGAGAEQISVLVRTIVESKASNNVLRYFYGIGYKLDHEVLKVGFAFRFQRGAQFTVTVTSANKMPKLHATDEAVAVTPGIQLVEITAPAAANNYNDVASSVTAFCEYLAPLLHLSKPGNSTGIVLTAGAAAASLMSSGGAKTL

SEQ ID NO: 44 - DNA, 654 bp, Carica papaya
  CDS (1)..(654): Carica papaya cDNA encoding Med18 protein
atggagtgtgtggttcaaggaattatagagacgcagcatgttgaggccctagagattcttcttcaaggcctttgtggggttcacagagaacgcttgagggtgcatgaaatctgcctaaaaagcgggcccaaccttggaattgtttcttcggaggttaggcttttgtgtgatcttgaacagcctgaacccacatggactgtcaaacatgttggaggcgcaatgcgtggtgctggtgcagagcaaatatcagttctggtaaggaccatggttgagagcaaagtgagcaagaatgttctccgcttattttatgcacttggctacaagttggaccatgagcttttgagagtgggattcaccttccatttccaaaggggtgctcagatcaaggtttctgtgtcatcaatcaataagatgctgaaactacatgcaacagacgaggctgtgccggtaaccccgggcatacagctagttgaagtgacggcccctgcaacctccgagaattatgcagaagttgtagctgcagtatcatctttttgtgaatatcttgcgccgctcttgcatttgtcgaaaccaggcatctcaacaggcgttgtcccgactgctgcagcagctgcagcatctcttatgtccgatggtggaggcacaaccttgtag

SEQ ID NO: 45 - protein, 217 aa, Carica papaya
MECVVQGIIETQHVEALEILLQGLCGVHRERLRVHEICLKSGPNLGIVSSEVRLLCDLEQPEPTWTVKHVGGAMRGAGAEQISVLVRTMVESKVSKNVLRLFYALGYKLDHELLRVGFTFHFQRGAQIKVSVSSINKMLKLHATDEAVPVTPGIQLVEVTAPATSENYAEVVAAVSSFCEYLAPLLHLSKPGISTGVVPTAAAAAASLMSDGGGTTL

SEQ ID NO: 46 - DNA, 654 bp, Cucumis sativus
  CDS (1)..(654): Cucumis sativus cDNA encoding Med18 protein
atggagtgtgtggttcagggcataattgaaacccagcacgttgaagcccttgagatacttcttcaaggtatttgcggcgttcctagagaaagattgaggctccatgaaatatgcctaaaaagtggacctagcctaggtgctgtagcatctgaggttcggatcttatgcgaccttgagcaagctgaaccgacatggacggttaaacatattggaggggcaattagaggtgcaggtgctgaacaactctctgttctggttaggacaatggtagaaagcaaagttagcaagaatgtgcttcgtttattctatgcacttggctataagttagatcatgagctgctgagagttggttttgccttccgcttccatcggggtgcccacataacagtgacagtttcatcagttaataagatgttaaagctacattcgactgatgaggctgtacctgtaacgcctggtatacagctggtggaagtgacagccccagcaacttctgataactattctgaagtagccggggcagtgtcgactttctgtgaatatcttgcaccgctgctgcatttgtcgaaaccaggaatatccacaggtgttgttcctactgctgcggcagctgcagcctctcttctatctgatggggcaggaacaacaatgtag

SEQ ID NO: 47 - protein, 217 aa, Cucumis sativus
MECVVQGIIETQHVEALEILLQGICGVPRERLRLHEICLKSGPSLGAVASEVRILCDLEQAEPTWTVKHIGGAIRGAGAEQLSVLVRTMVESKVSKNVLRLFYALGYKLDHELLRVGFAFRFHRGAHITVTVSSVNKMLKLHSTDEAVPVTPGIQLVEVTAPATSDNYSEVAGAVSTFCEYLAPLLHLSKPGISTGVVPTAAAAAASLLSDGAGTTM

SEQ ID NO: 48 - DNA, 654 bp, Eucalyptus grandis
  CDS (1)..(654): Eucalyptus grandis cDNA encoding Med18 protein
atggagtgtgtggtccaaggaattatagagacacagcatgttgaggcccttgagatccttctgcaaggtctttgtggtgttcaaaaagaacgcttgaggatccatgagctatgcctgaaaagcagtccaaatctaggtgcagtaccctcagaagttcaaatattgtgtgatctcgcgcaaccagaacctagttggactgttcggcatgttgggggctcaatgagaggcactggtgctgaacaaatttcagttcttgtgagacccatggtagaaagcaaagcgagcaagaatgtacttcgtttcttttatgctcttggctacaaattggaccatgagctgctgagagtgggatttgcctttcacttccagaggggcgttcagattaccgttactgtatcttcagtaaataagatgttaaaattacatgcgacggatgaggctgtgccagtgacacctggtatgcagctggttgaagtaactgctcctgcaacatcagagaactatactgaagtagttgcagctgtgacttcattttgtgaacatcttgctccgctcttgcacctgtcaaagccaggcatctcaactggggttgtgccaacagcggcggctgctgctgcgtctcttatgtctgatggtggaggtacaactttataa

SEQ ID NO: 49 - protein, 217 aa, Eucalyptus grandis
MECVVQGIIETQHVEALEILLQGLCGVQKERLRIHELCLKSSPNLGAVPSEVQILCDLAQPEPSWTVRHVGGSMRGTGAEQISVLVRPMVESKASKNVLRFFYALGYKLDHELLRVGFAFHFQRGVQITVTVSSVNKMLKLHATDEAVPVTPGMQLVEVTAPATSENYTEVVAAVTSFCEHLAPLLHLSKPGISTGVVPTAAAAAASLMSDGGGTTL

SEQ ID NO: 50 - DNA, 654 bp, Glycine max
  CDS (1)..(654): Glycine max cDNA 1 encoding Med18 protein
atggaatgcgtggttcagggaattatagagacacagcatgttgaggccttggagattcttcttcaaggactatgtggcgttcagagagaacgattgaggatccatgaaatatgcctcaagagtggtcaacatctaggtactgtagcctcagaggttcgactgttgtgtgacctagagcaagctgaaccttcgtggactgttaaacatgtaggtggtgcaatgaggggagcaggcgcagagcaaatttctgttctggttaggtcaatggtggaaagcaaaacaagcaagaatgcactgcgcatgttttatacactggggtacaagttagaccatgagctgttgagggtgggattttccttcaatttccacaggggtgcacaaatcactgtaacggtttcatcaatcaataaaatgctgaagctgcatgcaactgacgaggccgtgccagtaacacctggtatacaaatggttgaagtaacagcacctgcaactgctgaaacctacactgaagttgcggctgctgtgtcatccttttgtgaataccttgcaccgcttctccacctgtcgaaaccaggcatttcaaccggtgttgttcctacagctgctgcagctgctgcatctctgatgtctgacggtgggagtacaacgttgtag

SEQ ID NO: 51 - protein, 217 aa, Glycine max
MECVVQGIIETQHVEALEILLQGLCGVQRERLRIHEICLKSGQHLGTVASEVRLLCDLEQAEPSWTVKHVGGAMRGAGAEQISVLVRSMVESKTSKNALRMFYTLGYKLDHELLRVGFSFNFHRGAQITVTVSSINKMLKLHATDEAVPVTPGIQMVEVTAPATAETYTEVAAAVSSFCEYLAPLLHLSKPGISTGVVPTAAAAAASLMSDGGSTTL

SEQ ID NO: 52 - DNA, 729 bp, Glycine max
  CDS (1)..(729): Glycine max cDNA 2 encoding Med18 protein
atggatttatcaaaagtgttagattgcataaataatacatgcatctcgtgtgtaatgcagctgagcatagtggatatggaatgtgtggttcagggaattatagagactcagcatgttgaggccttggagattcttcttcaaggactatgtggtgttcagagagaacgattgaggatccatgaaatatgcctcaagagtggtccacatctgggcactgtagcctcagaggttcgactcttgtgtgacctagagcaagctgaaccttcctggactgttaaacatgtaggtggtgcaatgaggggagcaggtgcagagcaaatttctgttctcgttaggtcaatggtggaaagcaaaacaagcaagaatgcacttcgcatgttttatacactggggtacaagttagaccatgagctgctgagggtgggattttccttcaatttccacaggggtgcacaaatcactgtaacagtttcatcaatcaataaaatgctgaagctgcatgcaactgatgaggccgtgccagtaacacctggtatacaaatggttgaagtaacagcacctgcaactgctgaaacctacgctgaagttgcttctgctgtgtcatccttttgtgaataccttgcaccgcttctccacctgtccaaaccaggcatttcaaccggtgttgttcctactgctgctgcagctgctgcatctctaatgtctgatggtgggggtacaaatttgtag

SEQ ID NO: 53 - protein, 242 aa, Glycine max
MDLSKVLDCINNTCISCVMQLSIVDMECVVQGIIETQHVEALEILLQGLCGVQRERLRIHEICLKSGPHLGTVASEVRLLCDLEQAEPSWTVKHVGGAMRGAGAEQISVLVRSMVESKTSKNALRMFYTLGYKLDHELLRVGFSFNFHRGAQITVTVSSINKMLKLHATDEAVPVTPGIQMVEVTAPATAETYAEVASAVSSFCEYLAPLLHLSKPGISTGVVPTAAAAAASLMSDGGGTNL

SEQ ID NO: 54 - DNA, 645 bp, Glycine max
  CDS (1)..(645): Glycine max cDNA 3 encoding Med18 protein
atggaatgcgtggttcagggaattatagagacacagcatgttgaggccttggagattcttcttcaaggactatgtggtgttcagagagaacgattgaggatccatgaaatatgcctcaagagtggtccacatctgggtactgtagcctcagaggttcgactgttgtgtgacctagagcaagctgaaccttcgtggactgttatacatgcaggtggtgcaatgaggggagcaggcgcagagcaaatttctgttctggttaggtcaatggtggaaagcaaaacaagcaagaatgcactgcgcatgttttatacactggggtacaagttagaccatgagctgctgagggtgggattttccttcaatttccacaggggtgcacaaatcactgtaacggtttcatcaatcaataaaatgctgaagctccatgcaactgacgaggccatgccagtaacacctggtatacaaatggttgaagtaacagcacctgcaactgctgaaacctatgctgaagttgcgtctgctgtgtcatccttttgtgaataccttgcaccgcttctccacctgtcgaaaccaggcatttcaactggtgttgttcctgcagctgctgcatctctgatgtctgatggtgggggtacaacgttgtag

SEQ ID NO: 55 - protein, 214 aa, Glycine max
MECVVQGIIETQHVEALEILLQGLCGVQRERLRIHEICLKSGPHLGTVASEVRLLCDLEQAEPSWTVIHAGGAMRGAGAEQISVLVRSMVESKTSKNALRMFYTLGYKLDHELLRVGFSFNFHRGAQITVTVSSINKMLKLHATDEAMPVTPGIQMVEVTAPATAETYAEVASAVSSFCEYLAPLLHLSKPGISTGVVPAAAASLMSDGGGTTL

SEQ ID NO: 56 - DNA, 660 bp, Manihot esculenta
  CDS (1)..(660): Manihot esculenta cDNA encoding Med18 protein
atggagtgcgtggtacaagggattattgagacacagcatgttgaggcccttgagattcttcttcagggtctttgtggtgttcatagagaagatttcaggatccatgaactatgtctgaaaagtggtccaaaccttggacttgttgcttcagaagttcgacttttgtttgatctttaccaggctgaacctacttggacggttaaacatgttggaggtgcaatgagaggtgctggtgctgatcaaatctcagtcctggtgaggacaatggtagaaagcaaagcaagcaagaatgtgcttcgcttattttatgcacttggctataaattggaccatgagttgcttagggtgggatttgccttccatttccaaaggggtgctcggataactgtggctgtctcatctgttaataagatgctaaaactacatgcaactgatggtgacgaggcagtgccagttacccctggcatacaactggttgaagtaacggcccctgcaacatctgaaaattattctgaagttgttgctgcagtgtcatccttctgcgaatatcttgcaccgcttctgcatttgtcaaaaccaggtgtttcaacaggggttgttcctactgctgctgccgctgctgcatcccttatgtctgatggtggaggcaccaccttgtag

SEQ ID NO: 57 - protein, 219 aa, Manihot esculenta
MECVVQGIIETQHVEALEILLQGLCGVHREDFRIHELCLKSGPNLGLVASEVRLLFDLYQAEPTWTVKHVGGAMRGAGADQISVLVRTMVESKASKNVLRLFYALGYKLDHELLRVGFAFHFQRGARITVAVSSVNKMLKLHATDGDEAVPVTPGIQLVEVTAPATSENYSEVVAAVSSFCEYLAPLLHLSKPGVSTGVVPTAAAAAASLMSDGGGTTL

SEQ ID NO: 58 - DNA, 654 bp, Mimulus guttatus
  CDS (1)..(654): Mimulus guttatus cDNA encoding Med18 protein
atggagtgcgtggttcagggaatcatagagactaagcatgttgaggcactggagattctccttcaaggactctgtggcgttcatcgtgagagtttgagggttcacgaattatgtctgaaaagtgtcccaaatctcggattagtatcatcggaaatccgactcctatgcgatctcgagcaacccgaaccgacatggactgtgcgacacgttggtggtgcactgaggggagctggagcggatcaaatctctgtgcttgtgaggaatatgatggaaagtaaagtaagcaagaacgcgcttcgattattctatgctcttggatacaagctcgaccacgagctactgagagttgggttcaccttccaattccatagaggcgctcagataacagtgacagtttcttcggttaataaaatgctgaaactgcattcgaccgacgaagcagtgccggttacaccaggtatacagcttgttgaagtgactgcccctgcttcgtctgagaactataccgaagttgttgctgctatgtcttccttttgtgaatatcttgctccgttactgcatctatcgaaacccggtgtctcaactggagttgttccaacagctgccgcagctgccgcatctctaatgtcggacggtggaggaacggcactctaa

SEQ ID NO: 59 - protein, 217 aa, Mimulus guttatus
MECVVQGIIETKHVEALEILLQGLCGVHRESLRVHELCLKSVPNLGLVSSEIRLLCDLEQPEPTWTVRHVGGALRGAGADQISVLVRNMMESKVSKNALRLFYALGYKLDHELLRVGFTFQFHRGAQITVTVSSVNKMLKLHSTDEAVPVTPGIQLVEVTAPASSENYTEVVAAMSSFCEYLAPLLHLSKPGVSTGVVPTAAAAAASLMSDGGGTAL

SEQ ID NO: 60 - DNA, 663 bp, Oryza sativa
  CDS (1)..(663): Oryza sativa cDNA encoding Med18 protein
atggagtgcgtggtgcaggggatcatcgagacccagcatgttgatgctcttgaggttcttctccagggcctctctggtgtcccaaaagaacgtgtcagagtgcatgagctctgtcttaaaagtgggccaaatctaggagttgtcccatcagaggttcgtttattgtgtgacttagctcagtccacgccatcctggaccataagacatgttggaggtgccatgagaggtgctggtgcagagcaaatctcagtccttgtgcggtcaattgtagagagcaaagccagcaacaatgtattgcgttacttctacggtatagggtacaagttagatcatgaagttctgaagggaggatttgcatttcgtttccaccgaggtgcccagataactgtgaccgttacctctgttagcaagatgacaaaactccatgctaccaacgaagctgtgccaatcactcctgcaatacagctagtggaaatcacggcccctgctgctgctgacaattacaacgatgtcgtttcagccgtcacttcgttctgtgaatatctagcaccgcttctgcatctctctaaaccaggcaattcgactggaatcgtcccgactgcaggcgctgctgccgcctcactcatgtcaagtggtggtggtggtggtggtaaaacgttgtga

SEQ ID NO: 61 - protein, 220 aa, Oryza sativa
MECVVQGIIETQHVDALEVLLQGLSGVPKERVRVHELCLKSGPNLGVVPSEVRLLCDLAQSTPSWTIRHVGGAMRGAGAEQISVLVRSIVESKASNNVLRYFYGIGYKLDHEVLKGGFAFRFHRGAQITVTVTSVSKMTKLHATNEAVPITPAIQLVEITAPAAADNYNDVVSAVTSFCEYLAPLLHLSKPGNSTGIVPTAGAAAASLMSSGGGGGGKTL

SEQ ID NO: 62 - DNA, 651 bp, Populus trichocarpa
  CDS (1)..(651): Populus trichocarpa cDNA encoding Med18 protein
atggagtgtgtggtacagggaattatagagacacagcatgtagaggcccttgagattcttcttcaaggtctttgtggtgttcataaagaacacctacgggtccatgaattataccttaaaagtggtccaaaccttgggcatgtgacttcagaggttcgactgttgtgtaatcttgagcatcctgaaccctggacggtcaaacatgttgggggtgcactgaggggtgctggtgctgagcaaatctcagttctggtcaggaatatggtagaaagcaaagcaagcaagaatgtgcttcgattgttttatgcacttggctacaaattagaccatgagttgctaagagttggatccgccttccatttcaaaaggggtgcttggataactgtcacagtgtcttctattaataagatgctaaaactacatgccattgatgatactatgccagtaacccttggtatacaggtggtagaagtaacagctcctgcaacgtctgaaaattattccgaagttgctgctgcagtgtcgtctttctgcgaatatcttgcaccgcttctgcacttgtcaaaaccaggtgtttcaacaggggttgttcctaccgccgcagccgccgctgcatctcttatgtcagatggtggaggcacaaccttgtag

SEQ ID NO: 63 - protein, 216 aa, Populus trichocarpa
MECVVQGIIETQHVEALEILLQGLCGVHKEHLRVHELYLKSGPNLGHVTSEVRLLCNLEHPEPWTVKHVGGALRGAGAEQISVLVRNMVESKASKNVLRLFYALGYKLDHELLRVGSAFHFKRGAWITVTVSSINKMLKLHAIDDTMPVTLGIQVVEVTAPATSENYSEVAAAVSSFCEYLAPLLHLSKPGVSTGVVPTAAAAAASLMSDGGGTTL

SEQ ID NO: 64 - DNA, 654 bp, Ricinus communis
  CDS (1)..(654): Ricinus communis cDNA encoding Med18 protein
atggaatgcgtggtacaaggaattatcgagacacagcatgttgaggccctcgagattcttcttcagggtctttgtggtgttcatagagaacgcttaaggatccatgaattatgtctgaaaagtggtccaaaccttggaatagttgcttcagaggttcgacttctgtgtgatctggaacagcctgaacctacctggactgttaaacatgttggaggtgcaatgaggggtgctggtgccgagcaaatctcagtcctggtaaggactatggtggaaagcaaagcaagcaagaatgtgcttcgcttattttatgcacttggctacaaattggatcatgagttgctgagagtgggatttgccttccatttccagaggggtgctcggataactgttcgggtctcatctgttaataaaatgctaaaactacatgcaactgatgaggctgtaccagtaactcctggcattcaactggttgaagtaacagcccccgcaacacctgaaaattactctgaagttgttgcagcagtatcatccttctgtgaatatcttgctccgcttctgcatttatcaaaaccaggtgtttcaaccggggttgtccctactgctgctgctgcagctgcatctcttatgtcggacggtggaggcacaaccttgtag

SEQ ID NO: 65 - protein, 217 aa, Ricinus communis
MECVVQGIIETQHVEALEILLQGLCGVHRERLRIHELCLKSGPNLGIVASEVRLLCDLEQPEPTWTVKHVGGAMRGAGAEQISVLVRTMVESKASKNVLRLFYALGYKLDHELLRVGFAFHFQRGARITVRVSSVNKMLKLHATDEAVPVTPGIQLVEVTAPATPENYSEVVAAVSSFCEYLAPLLHLSKPGVSTGVVPTAAAAAASLMSDGGGTTL

SEQ ID NO: 66 - DNA, 654 bp, Sorghum bicolor
  CDS (1)..(654): Sorghum bicolor cDNA encoding Med18 protein
atggagtgcgtggtgcagggaatcatcgagacccagcatgtcgaagctctggaggttcttctccagggtctctctggtgtcccaaaagagcgtgttagggtgcatgagctctgccttaaaagtggacccaatctaggtgttgtcccatcggaggttcgtttgttgtgtgacttagctcagcccacaccatcctggaccataagacatgttggtggtgccatgagaggtgctggtgcagagcaaatctcagttctggttcgtacaattgtggagagcaaagctagcaagaacgtcttgcactacttctacactctagggtacaagctagaccatgaactcttgaagattggttttgcgtttcgtttccaccgaggtgcccaaatcactgtcactgttacatctactaacaagatgccaaggctccatgctaccgatgaggctgtgccagtcactcctggcatacagttagtggaaatcacagcccctgctgctgctgataattacaatgatgttgtttcagctgtctctgcattctgtgaatatcttgccccgcttctgcatctgtcaaaaccaggacactcgactggaattgttgcaactgcaggcgctgctgctgcctcactcatgtcaagtggtggtggcaaaaccttgtaa

SEQ ID NO: 67 - protein, 217 aa, Sorghum bicolor
MECVVQGIIETQHVEALEVLLQGLSGVPKERVRVHELCLKSGPNLGVVPSEVRLLCDLAQPTPSWTIRHVGGAMRGAGAEQISVLVRTIVESKASKNVLHYFYTLGYKLDHELLKIGFAFRFHRGAQITVTVTSTNKMPRLHATDEAVPVTPGIQLVEITAPAAADNYNDVVSAVSAFCEYLAPLLHLSKPGHSTGIVATAGAAAASLMSSGGGKTL

SEQ ID NO: 68 - DNA, 651 bp, Vitis vinifera
  CDS (1)..(651): Vitis vinifera cDNA encoding Med18 protein
atggagtgcgttgttcagggaatcatagagacccagcatgttgaggcccttgagatccttctccaaggtctttgtggtgttcatagagaacgattaaggatccatgaattgtgcctcaaaagtggcccaaacctcgggcttgtagcttcagaggttcgagtattatgtgatcttgagcagccagaacctacttggatagttcggcatattgggggtgccatgagaggttctggtgctgaacaaatttcagttctggtgaggactatggtggaaagcaaagtaagcaagaatgtattgcgcttattttatgcacttggctacagattggatcatgaattgctgagagtaggattcgccttccattttcaaaggggtgcttcaattacagttactgtgtcgtcagtcaataagatgcttaaattacatgcaactgatgaggctgtgccagtgacacctggtatacagctggttgaagtgacagcccctgcatcttccgaaaactatagtgaagttgctgctgctgtgtcatccttctgtgaatatcttgcaccgcttctgcatttatcaaaacctggtacctcaacaggagttgtccctactgctgctgcagctgcagcatctctcatgtcagatgctggggttacaacatga

SEQ ID NO: 69 - protein, 216 aa, Vitis vinifera
MECVVQGIIETQHVEALEILLQGLCGVHRERLRIHELCLKSGPNLGLVASEVRVLCDLEQPEPTWIVRHIGGAMRGSGAEQISVLVRTMVESKVSKNVLRLFYALGYRLDHELLRVGFAFHFQRGASITVTVSSVNKMLKLHATDEAVPVTPGIQLVEVTAPASSENYSEVAAAVSSFCEYLAPLLHLSKPGTSTGVVPTAAAAAASLMSDAGVTT

SEQ ID NO: 70 - DNA, 654 bp, Zea mays
  CDS (1)..(654): Zea mays cDNA encoding Med18 protein
atggagtgcgtggtgcagggaatcatcgagaaccagcatgtcgatgctcttgaggttctactccagggactctctggtgtcccgaaagagcgttttagggtgcatgagctctgccttaaaagtgggcccaatctaggtgttgtcccatcggaggttcgtttgttgtgtgacttagctcagcccacaccatcctggaccataagacatgttggtggtgccatgagaggtgctggtgcggagcaaatctcagttctggttcgtacagttgtggagagcaaagctagcaagaacgtcttgcactacttctacaccctagggtacaagctagaccatgaactcttgaagagcggttttgcatttcgtttccaccgaggtgcccaaatcactgtcactgttacatctgctaacaagatgtcaaggctccatgcaaccgatgaagctacgccagtcactcctgccatacagctagtggaaatcacagctcctgctgctgctgataattacaatgatgttgtttcagctgtcactgcgttctgtgaatatcttgccccgctgctgcatctgtcaaaaccaggacactcgacaggaatcgttccaacagcagccgctgctgctgcctcactcatgtcaagtggcggtggcaaaaccttgtaa

SEQ ID NO: 71 - protein, 217 aa, Zea mays
MECVVQGIIENQHVDALEVLLQGLSGVPKERFRVHELCLKSGPNLGVVPSEVRLLCDLAQPTPSWTIRHVGGAMRGAGAEQISVLVRTVVESKASKNVLHYFYTLGYKLDHELLKSGFAFRFHRGAQITVTVTSANKMSRLHATDEATPVTPAIQLVEITAPAAADNYNDVVSAVTAFCEYLAPLLHLSKPGHSTGIVPTAAAAAASLMSSGGGKTL

SEQ ID NO: 72 - DNA, 60 bp, Arabidopsis thaliana
  misc_structure (1)..(60): AtMed25-EcoRI-aa551-fwd  primer
ggggacaagtttgtacaaaaaagcaggctccgaattcacttcacaatccaaatatgtgaa

SEQ ID NO: 73 - DNA, 62 bp, Arabidopsis thaliana
  misc_structure (1)..(62): AtMed25-SalI-aa680-rev primer
ggggaccactttgtacaagaaagctgggtggtcgacttaatttggaatttgtggtttaaaca

SEQ ID NO: 74 - DNA, 53 bp, Arabidopsis thaliana
  misc_structure (1)...(53): Gateway cloning primer for Med18 RNAi constructs KR939_F1_attB2
ggggaccactttgtacaagaaagctgggtgcaagcaagaatgtgcttagattg

SEQ ID NO: 75 - DNA, 54 bp, Arabidopsis thaliana
  misc_structure (1)...(54): Gateway cloning primer for Med18 RNAi construct KR939_R1_attB1
ggggacaagtttgtacaaaaaagcaggctaaacacctggttttgacaagtgcag

SEQ ID NO: 76 - DNA, 49 bp, Arabidopsis thaliana
  misc_structure (1)...(49): Gateway cloning primer for Med18 RNAi constructs KR940_F1_attB2
ggggaccactttgtacaagaaagctgggtaggggttgttcctactgccg

SEQ ID NO: 77 - DNA, 52 bp, Arabidopsis thaliana
  misc_structure (1)...(52): Gateway cloning primer for Med18 RNAi constructs KR940_R1_attB1
ggggacaagtttgtacaaaaaagcaggctcccagcatcaagcggataactag

SEQ ID NO: 78 - DNA, 53 bp, Arabidopsis thaliana
  misc_structure (1)...(53): Gateway cloning primer for Med25 RNAi constructs KR941_F1_attB2
gggaccactttgtacaagaaagctgggtaggtgtctcttctggtatgaacacg

SEQ ID NO: 79 - DNA, 54 bp, Arabidopsis thaliana
  misc_structure (1)...(54): Gateway cloning primer for Med25 RNAi constructs KR941_R1_attB1
ggggacaagtttgtacaaaaaagcaggcttggtaactggattactgcacaaagc

SEQ ID NO: 80 - DNA, 51 bp, Arabidopsis thaliana
  misc_structure (1)...(51): Gateway cloning primer for Med25 RNAi constructs KR942_F1_attB2
ggggaccactttgtacaagaaagctgggtgaatgacgtcttctgtgcctgc

SEQ ID NO: 81 - DNA, 51 bp, Arabidopsis thaliana
  misc_structure (1)...(51): Gateway cloning primer for Med25 RNAi constructs KR942_R1_attB1
ggggacaagtttgtacaaaaaagcaggcttgcacccattccactttgtacc

SEQ ID NO: 82 - DNA, 433 bp, Arabidopsis thaliana
  iDNA (1)..(433): Med 25 RNAi construct sequence KR941
aggtgtctcttctggtatgaacacgatgattcctacccctggaatgtctcaacaagtacaatccgggatgcagcctcttggtgcaaacagtaactcagcagataatttgccaatgtcacgacaaacagcagggggcttgcaaccagcacaatcgaaatatgtcaaagtctgggagggaaacttgtctgggcaacggcaagggcagcctgtctttatcaccagattagaaggttataggagtgcgtcggcctctgagacgcttgcagcaaactggccaccgacaatgcaaatagttcggcttatatctcaggaccatatgaataacaagcaatatgtcgggaaggctgattttctagtttttcgggcaatgaatcagcatggatttcttggacaactacaagagaagaagctttgtgcagtaatccagttacca

SEQ ID NO: 83 - DNA, 306 bp, Arabidopsis thaliana
  iDNA (1)..(306): Med 25 RNAi construct KR942
gaatgacgtcttctgtgcctgctgctcaaactgtattttcatctgggcaaccaggatgtttcttcaataacaggttcagggaatcttactgggacaacacaaatcacaccaaactctggtctgggctcattcacttccgcaacctctaacatgcctggaaactcaaaccttggggggatttcacaaccaatgggtaatcttcacgcaggtgttagcatcagccaatcagcaatgagtggtctgggtgcaaacccaaacacaatgagtggttcaggagcacaaatggtacaaagtggaatgggtgca

SEQ ID NO: 84 - DNA, 301 bp, Arabidopsis thaliana
  iDNA (1)..(301): Med 18 RNAi construct KR939
gcaagcaagaatgtgcttagattgttttatgcacttggctacaaattagaccatgagttgctaagagtcggatccgccttccatttcaaaaggggtgcttggataactgtcacagtgtcttctattaataagatgctaaaactacatgccattgatgatactgtgccagtaacccttggtatacaggtggtagaagtaacagctcctgcaacgtctgaaaattattccgaagttgctgctgcagtgtcgtctttctgcgaatatcttgcaccgcttctgcacttgtcaaaaccaggtgttt

SEQ ID NO: 85 - DNA, 336 bp, Arabidopsis thaliana
  iDNA (1)..(336): Med 18 RNAi construct KR940
aggggttgttcctactgccgccgccgccgccgcatctcttatgtcagatggtggaggcacaaccttgtagcaatgttgtagagttccatgactgagcaaaaggcttcttttgaactgacggagcacaaactgtccacgctccatgtaacagattcatggtttttggtcctggacccccttcttttcctctgttggaaaattctcttgcaaggttgatgaaaaactgaagcagctgagttacaataacattaggggtactgtaattatttgtgttcatttgcaggattatgagttatgaggtggattgtatatactagttatccgcttgatgctggg

SEQ ID NO: 86 - DNA, 24 bp, Populus tremula x Populus tremuloides
  primer_bind (1)..(24): Med18 transcript detection forward primer - med18POP-940
actgtccacgctccatgtaacaga

SEQ ID NO: 87 - DNA, 24 bp, Populus tremula x Populus tremuloides
  primer_bind (1)..(24): M18 transcript detection reverse primer - med18POP-940
acaaatccacctcataactcataa

SEQ ID NO: 88 - DNA, 23 bp, Populus tremula x Populus tremuloides
  protein_bind (1)..(23): Med18 transcript detection forward primer - med18POP
agatgctaaaactacatgcattg

SEQ ID NO: 89 - DNA, 24 bp, Populus tremula x Populus tremuloides
  primer_bind (1)..(24): Med18 transcript detection reverse primer - Med18POP
cggtgcaagatattcgcagaaaga

SEQ ID NO: 90 - DNA, 21 bp, Populus tremula x Populus tremuloides
  primer_bind (1)..(21): Med25 transcript detection forward primer - med25-Pt942
aactgtattttcatctgggca

SEQ ID NO: 91 - DNA, 21 bp, Populus tremula x Populus tremuloides
  primer_bind (1)..(21): Med25 transcript detection reverse primer - med25-Pt942
cagaccactcattgcgattgg

SEQ ID NO: 92 - DNA, 24 bp, Populus tremula x Populus tremuloides
  primer_bind (1)..(24): Med25 transcript detection forward primer - med25POP
agatgctaaaactacatgccattg

SEQ ID NO: 93 - DNA, 24 bp, Populus tremula x Populus tremuloides
  primer_bind (1)..(24): Med25 transcript detection reverse primer - med25POP
agcaatgtctgagatggtaactgg

SEQ ID NO: 94 - DNA, 21 bp, Populus tremula x Populus tremuloides
  primer_bind (1)..(21): 18S RNA detection forward primer - 18S
ctatcaactttcgatggtagg

SEQ ID NO: 95 - DNA, 22 bp, Populus tremula x Populus tremuloides
  primer_bind (1)..(22): 18S RNA detection reverse primer - 18S
ccgtgtcaggattgggtaattt